(19) United States
(12) Patent Application Publication
(10) Pub. No.: US 2016/0051514 A1
(43) Pub. Date: Feb. 25, 2016

(54) METHODS FOR TREATMENT OF CARDIOMYOPATHY

(71) Applicants: THE BRIGHAM AND WOMEN'S HOSPITAL, INC., Boston, MA (US); BETH ISRAEL DEACONESS MEDICAL CENTER, INC., Boston, MA (US)

(72) Inventors: Calum MACRAE, Wellesley Hills, MA (US); Jeffrey SAFFITZ, Waban, MA (US)

(21) Appl. No.: 14/782,841

(22) PCT Filed: May 2, 2014
(86) PCT No.: PCT/US2014/036593

§ 371 Date: Oct. 7, 2015

Related U.S. Application Data
(60) Provisional application No. 61/819,174, filed on May 3, 2013.

(51) Int. Cl. A61K 31/404; A61K 31/4015; G01N 33/68; C07K 14/47

(52) U.S. Cl. CPC A61K 31/404; C07K 14/47; A61K 31/4015; G01N 33/6887; G01N 2333/47; G01N 2800/325

(57) ABSTRACT

The methods and assays described herein related to the diagnosis, prognosis and treatment of cardiomyopathy, e.g. arrhythmia. As described herein, the inventors have discovered that certain compounds can be used to treat cardiomyopathies. These compounds are demonstrated herein to reduce expression of nppb, reduce bradycardia, improve contractility, and increase survival. Notably, provided herein is data demonstrating that administration of these compounds can both prevent the development of symptoms when administered prophylactically and reduce symptom severity in subjects where it is administered after the development of symptoms.

### CROSS-REFERENCE TO RELATED APPLICATIONS

This application claims benefit under 35 U.S.C. §119(e) of U.S. Provisional Application No. 61/819,174 filed May 3, 2013, the contents of which are incorporated herein by reference in their entirety.

### GOVERNMENT SUPPORT

This invention was made with federal funding under Grant Nos. R01 HL100110, R01 HL102361, R01 HL113006, U01 HL099776, and R01 HL109264 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Apr. 25, 2014, is named 043214-077971-PCT_SEtxt and is 14,827 bytes in size

### TECHNICAL FIELD

The technology described herein relates to methods and assays relating to the diagnosis and treatment of cardiomyopathy.

### BACKGROUND

Many heart diseases are characterized by arrhythmia, including arrhythmogenic cardiomyopathy (AC), which is a highly arrhythmogenic form of human heart disease and a significant cause of sudden death in the young (Basso et al. Lancet 2009 373:1289-1300; Saffitz Annu Rev Pathol 2011: 299-321). Degeneration of cardiac myocytes and replacement by fibrofatty scar tissue marks disease progression, but arrhythmias are the cardinal feature of AC. Rhythm disturbances are usually the earliest manifestation of disease and often precede structural remodeling of the myocardium. Accordingly, understanding and targeting the sources of arrhythmia can permit the treatment of the underlying source of cardiomyopathies. AC patients with demonstrated or strongly suspected risk of sudden death usually receive implantable defibrillators, because there are no mechanism-based therapies to prevent arrhythmias or limit myocardial injury in this disease spectrum.

### SUMMARY

As described herein, the inventors have discovered that certain compounds can be used to treat cardiomyopathies. These compounds are demonstrated herein to reduce expression of nppb, reduce bradycardia, improve contractility, and increase survival. Notably, provided herein is data demonstrating that administration of these compounds can both prevent the development of symptoms when administered prophylactically and reduce symptom severity in subjects where it is administered after the development of symptoms.

Surprisingly, while the compounds described herein are GSK3 inhibitors, GSK3 inhibitors are not, as a class, efficacious in the treatment of cardiomyopathies. For example, the GSK3 inhibitors BIO (6-bromoindirubin-3′-oxime) and CHIR99021 do not exhibit the therapeutic benefits seen following the administration of compounds described herein, e.g. SB216763 and SAB415286. Without wishing to be bound by theory, it is possible that the compounds described herein (e.g. SB216763 and SAB415286) possess a novel activity not shared by GSK3 inhibitors generally.

In one aspect, described herein is a method of treating cardiomyopathy, the method comprising administering a compound of Formula I:

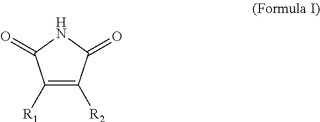

wherein R₁ is optionally substituted heteroaromatic or —NR₂R₃; R₂ is optionally substituted aryl; and R₃ is H or alkyl. In some embodiments, the cardiomyopathy is selected from the group consisting of arrhythmia; arrhythmogenic cardiomyopathy (AC); ischemic heart disease; heart failure; Naxos disease (ARVC); sarcoidosis and giant cell myocarditis. In some embodiments, the compound of Formula I is selected from the group consisting of SB216763 and SAB415286.

In one aspect, described herein is an assay comprising determining the level of SAP97 in a cardiomyocyte obtained from a subject wherein a decreased level of SAP97 in the cardiomyocyte, as compared to a reference level indicates the subject has cardiomyopathy or is at risk of developing cardiomyopathy. In one aspect, described herein is a method of treatment for cardiomyopathy, the method comprising administering a treatment for cardiomyopathy to a subject determined to have a decreased level of SAP97 in a cardiomyocyte, as compared to a reference level. In some embodiments, the treatment comprises administering a composition of Formula I:

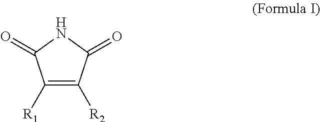

wherein R₁ is optionally substituted heteroaromatic or —NR₂R₃; R₂ is optionally substituted aryl; and R₃ is H or alkyl. In some embodiments, the treatment comprises administering SB216763 or SAB415286. In some embodiments, the level of SAP97 polypeptide present in the membrane is the level of SAP97 polypeptide located in the membrane of a cardiomyocyte. In some embodiments, the level of SAP97 polypeptide present in the membrane is the level of SAP97 polypeptide located at cell-cell junctions. In some embodiments, the cardiomyopathy is arrhythmogenic cardiomyopathy (AC); sarcoidosis; or giant cell myocarditis.

### BRIEF DESCRIPTION OF THE DRAWINGS

FIG. 1 depicts a graph of gene expression in the larval stages.

FIG. 2 depicts a graph of conduction/impulse propagation.

FIG. 3 depicts images demonstrating cardiomegaly and peripheral edema in fish.

FIG. 4 depicts a graph demonstrating sodium channel loss in zebrafish ventricular myocytes.

FIG. 5 depicts a graph demonstrating the decreask of peak INa in zebrafish ventricular myocytes in the Naxos (Nx) model.

FIG. 6 depicts a graph of the effect of SB216763 on development of cardiomyopathy in the zebrafish model. The two left-most bars represent control animals, and the x-axis represents the percentage of fish with cardiomyopathy.

FIG. 7 depicts a graph of the effect of SB216763 on peak INa in zebrafish ventricular myocytes.

FIGS. 8A-8D. FIG. 8A depicts a western immunoblot showing equivalent levels of expression of 2057del2 plakoglobin and endogenous plakoglobin in transfected NRVM cultures. 2057del2 plakoglobin migrates at a lower molecular weight than the wild-type protein. GAPDH was used as a loading control. FIG. 8B depicts graphs of TUNEL labeling of 2057del2 plakoglobin-expressing NRVMs under rest conditions and after 4 hr of uniaxial cyclical stretch. Non-transfected cultures were subjected to the same stretch protocol and used as controls. The average number of TUNEL-stained nuclei in 5 microscopic fields and the mean levels of caspase-3 activity are plotted on the graphs. 2057del2-plakoglobin expressing cells show significantly higher levels of apoptosis at rest compared to control myocytes (* p<0.05). Stretch greatly exacerbates apoptosis in myocytes expressing 2057del2 plakoglobin but not in controls compared to resting conditions (** p<0.05). FIG. 8C depicts graphs of TUNEL labeling of NRVMs expressing 2057del2 plakoglobin under resting conditions and following 4 hr of stretch in the presence or absence of pifithrin A. PifithrinA significantly reduced apoptosis in transfected myocytes exposed to stretch compared to cultures that were stretched in the absence of the inhibitor (# p<0.05). FIG. 8D depicts cytokine expression profiles. 24 hr post- transfection, 2057del2 plakoglobin-expressing myocytes secreted higher amounts of selected pro-inflammatory cytokines including IL-6 (second column of boxes from the left), TNFα(rightmost column of boxes), MIP-1α (left-most boxes) and RANTES (ovals) compared to control, non-transfected myocytes.

FIGS. 9A-9C. FIG. 9A depicts a graph of representative action potential (AP) tracings from a zebrafish ventricular myocyte expressing 2057del2 plakoglobinb (dashed line) or a control fish myocyte measured at 5 weeks post-fertilization. FIG. 9B depicts a graph of action potential upstrokes and first-time derivatives (dV/dt) in zebrafish myocytes expression 2057del2 plakoglobin vs. control myocytes at enlarged time scale. FIG. 9C depicts a graph of representative AP tracings in a neonatal rat ventricular myocyte expressing 2057del2 plakoglobin vs. a control myocyte showing a positive shift in resting potential, decreased upstroke velocity and action potential prolongation (note virtually identical changes to those seen in zebrafish myocyte action potentials).

FIGS. 10A-10C depict graphs of changes in I_{Na} current density in zebrafish myocytes expressing 2057del2 plakoglobin: (FIG. 10A), original traces; (FIG. 10B) dependence of I_{Na} on membrane potential (white circles: 2057del2 plakoglobin; black: control); (FIG. 10C) steady-state activation (quadrangles) and inactivation (triangles) curves (closed symbols: 2057del2 plakoglobin; open symbols: controls). The Boltzman fit to individual experiments used to calculate the V0.5 values for steady state activation and inactivation (mean±SE; white symbols) showed no significant effect of 2057del2 plakoglobin expression. FIGS. 10D-10F depict graphs of changes in I_{Ki} current density in zebrafish myocytes expressing 2057del2 plakoglobin: (FIG. 10D), original traces obtained after subtraction of the Ba²⁺-insensitive component; (FIG. 10E), kᵢ current at −100 mV; (FIG. 10F) kᵢ slope of linear portion between −100 and −60 mV.

FIG. 11A depicts western immunoblots showing the total cellular content of plakoglobin and Cx43 in NRVMs in the presence or absence of SB216763. SB216763 had no apparent effect on plakoglobin expression levels, while total Cx43 expression was increased in both transfected and control myocyte cultures. GAPDH was used as a loading control. FIG. 11B depicts a graph of TUNEL labeling of 2057del2 plakoglobin-expressing NRVMs in the presence or absence of SB216763. Non-transfected cells were used as controls. The average number of TUNEL-stained nuclei in 5 microscopic fields is plotted on the graph. 2057del2 plakoglobin-expressing cells showed significantly higher levels of apoptosis at rest compared to control myocytes (* p<0.05). SB216763 significantly reduced the mean apoptosis index in 2057del2 plakoglobin-expressing myocytes (** p<0.05). FIG. 11C depicts cytokine expression profiles. 24 hr post-transfection, 2057del2 plakoglobin-expressing NRVMs secreted higher amounts of pro-inflammatory cytokines into the culture medium including IL-6, TNFα, MIP-1α and RANTES. Exposure of transfected cultures to SB216763 for 24 hr had no apparent effect on the cytokine profile. 48 hr post-transfection, IL-6, TNFα, MIP-1α and RANTES levels appeared reduced compared to 24 h post-transfection. However, transfected myocytes secreted higher levels of additional cytokines including fractalkine, IFNγ, MIP-1γ, IP-10 and IL-1ra. Exposure to SB216763 for 48 hr restored the cytokine profile to control levels.

FIGS. 12A-12E demonstrate the effects of SB216763 on action potential parameters (12A-12C) and I_{Na} and I_{Ki} current densities (12E,12F) in zebrafish ventricular myocytes; controls: black; myocytes expressing 2057del2 plakoglobin: white; myocytes expressing 2057del2 plakoglobin and treated with SB216763: textured.

FIG. 13 depicts western immunoblots showing the total cellular content of SAP97 and Nav1.5 in control myocytes and myocytes expressing 2057del2 plakoglobin in the presence or absence of SB216763. GAPDH was used as a loading control. Changes in the distribution of immunostaining signal occured without an apparent change in total cellular content of SAP97 and Nav1.5.

FIG. 14 is a diagram of an exemplary embodiment of a system for performing an assay for determining the level of SAP97 in the membrane of a cardiomyocyte in a sample obtained from a subject.

FIG. 15 is a diagram of an embodiment of a comparison module as described herein.

FIG. 16 is a diagram of an exemplary embodiment of an operating system and instructions for a computing system as described herein.

FIGS. 17A-17K demonstrate a zebrafish model of AC and chemical screen. Representative images of a 5-week-old control sibling (FIG. 17A) and 2057del2 plakoglobin (PG) zebrafish (FIG. 17B) (scale bar=1 mm), dissected hearts (A′ and B′ control sibling and2057del2 plakoglobin, respectively; scale bar=200 μm; OFT, out-flow tract; a, atrium; v, ventricle) and H&E stained sections (A″ and B″, control sibling and 2057del2 plakoglobin (PG) mutant fish respectively; scale bar=200 m; OFT, out-flow tract; a, atrium; v, ventricle) showing cardiomegaly, wall thinning and chamber dilatation in early adulthood are presented. FIG. 17C depicts survival curves for control and 2057del2 plakoglobin mutant fish. Data were pooled from three independent experiments and presented as total percentage fish survival as a function of time. FIG. 17D depicts ventricle/body size ratios in control sibling versus 2057del2 plakoglobin mutants at 5 weeks of age. FIG. 17E depicts heart rate (Bpm, beats per minute); FIG. 17F depicts stroke volume (diastolic volume minus systolic volume; n1); and FIG. 17G depicts cardiac output (stroke volume×heart rate; nL/min) in control sibling vs. 2057del2 plakoglobin fish measured in 48hpf larvae; mean±SD; *p<0.05). FIG. 17H depicts the results of quantitative RT-PCR showing 2-fold induction of the cardiac natriuretic peptide BNP in NAXOS embryos compared to control siblings at 72 hpf; mean±SD; * P<0.01. FIG. 17I depicts the results of an experiment using a luciferase-based reporter for cardiac natriuretic peptide expression in control siblings (BNP-LUC) and 2057del2 plakoglobin (BNP-LUC 2057del2 PG) embryos at 72hpf; mean luciferase reading per embryos±SEM; *P<0.01. FIG. 17J depicts the BNP luciferase activity of BNP-LUC 2057del2 PG embryos (72hpf) after DMSO and SB216763 (SB2) treatment; mean luciferase reading per embryos±SEM; *P<0.01. FIG. 17K depicts percent survival of untreated 2057del2 plakoglobin fish and 2057del2 plakoglobin fish treated with SB216763 (SB). SB216763 was added to the water at 24 hpf and washed out at 6 days. Fish were put on regular flow for an additional 4 weeks and survival was counted; data pooled from three independent experiments; *p<0.05.

FIG. 18 depicts Western immunoblots showing equivalent levels of 2057del2 plakoglobin and endogenous plakoglobin in transfected NRVM cultures. 2057del2 plakoglobin migrates at a lower molecular weight than the wild-type protein. GAPDH was used as a loading control.

FIGS. 19A-19F demonstrate the Ga14/UAS-based expression system and chemical screen protocol in zebrafish. FIG. 19A depicts a diagram of the the driver construct, which contains the cardiac specific promoter cmlc driving the expression of Ga14-VP 16, which binds to the UAS on the responder construct, expressing the 2057del2 plakoglobin (PG) mutation in human plakoglobin. Each construct has a fluorescent marker (driver: green heart; responder: red eyes) allowing efficient identification of transgenic lines as well as simultaneous in vivo monitoring of pathology and phenotypes. FIGS. 19B-19C depict transmission electron micrographs of the ventricular wall of a control sibling (FIG. 19B) and a 2057del2 plakoglobin mutant fish (FIG. 19C) (7 dpf) showing widened gaps between adjacent cells (arrow); scale bar=500 nm. FIG. 19D depicts myocardial glycogen content in controls and 2057del2 plakoglobin mutant fish. FIGS. 19E-19F depict the design and outline of the chemical screen assay for compounds that suppressed the disease response of the BNP-LUC 2057del2 plakoglobin mutant in intact zebrafish embryos. A long half-life luciferase reagent and high sensitivity luminescence microplate reader were used to rapidly assess nppb::F-Luciferase expression without the need to homogenize zebrafish embryos.

FIGS. 20A-20B demonstrate that there is no change in IKr current density in ventricular myocytes from controls and mutant fish at 3-4 weeks post-fertilization. FIG. 20A dpicts an original recording of tail current with repolarizing steps from 20 to −40 mV. That this current is due to IKr is shown by complete inhibition by the specific blocker E4031. FIG. 20A demonstrates the dependence of IKr tail currents on membrane potential in zebrafish ventricular myocytes. There is no difference between control cells and cells expressing 2057del2 plakoglobin.

FIGS. 21A-21D demonstrate decreased INa and IK1 current density in neonatal rat ventricular myocytes expressing 2057del2 plakoglobin, and rescue by SB216763. FIG. 21A depicts a graph of the dependence of INa current density on membrane potential in control myocytes and myocytes expressing 2057del2 plakoglobin (PG). FIG. 21B depicts a graph of INa current density in control myocytes and myocytes expressing 2057del2 plakoglobin (PG) in the presence or absence of SB216763. FIG. 21C depicts a graph of the dependence of IK1 current density on membrane potential in control myocytes and myocytes expressing 2057del2 plakoglobin (PG). FIG. 21D depicts a graph of IK1 current density at a membrane potential of −100 mV in control myocytes and myocytes expressing 2057del2 plakoglobin (PG) in the presence or absence of SB216763. Numbers above bars indicate “n” for each condition; * p<0.001 vs. both control myocytes and SB216763-treated myocytes expressing 2057del2 plakoglobin.

### DETAILED DESCRIPTION

As described herein, the inventors have demonstrated that certain GSK3 inhibitors (but not all GSK3 inhibitors) can be used to treat cardiomyopathy, e.g. arrhythymic cardiomyopathy. Accordingly, described herein are methods for the treatment of cardiomyopathies and the use of certain compounds for the treatment of cardiomyopathy.

In one aspect, described herein is a method of treating cardiomyopathy, the method comprising administering a compound of Formula I.

In some aspects of the invention, the inventive compounds have structural formula (I):

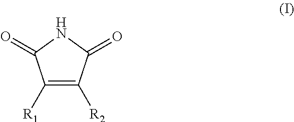

wherein: R₁ is optionally substituted heteroaromatic or —NR₂R₃;
R₂ is optionally substituted aryl; and
R₃ is H or alkyl.

In some embodiments, R₁ is

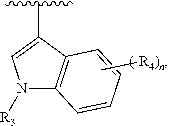

wherein R₄ is selected from halogen, hydroxyl, NO₂, C₁₋₄ alkyl, or C₁₋₄ alkoxyl and n is an integer 0-4 inlcusive.

In some embodiments, R₁ is

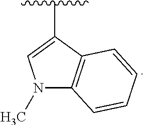 [SMILES: *c1cn(C)c2ccccc12]

In some embodiments, R₂ is unsubstituted aryl. In some embodiments, R₂ is substituted aryl. In some embodiments, R₂ is unsubstituted phenyl. In some embodiments, R₂ is substituted phenyl. In some embodiments, R₂ is substituted the ortho position. In some embodiments, R₂ is substituted at the meta position. In some embodiments, R₂ is substituted at the para position. In some embodiments, R₂ is substituted at more than one position. In some embodiments, R₂ is substituted at two positions. In some embodiments, R₂ is substituted at threee positions or more. In some embodiments, R₂ is substituted with a C₁₋₄ alkyl. In some embodiments, R₂ is substituted with C₁ alkoxy. In some embodiments, R₂ is substituted with NO₂. In some embodiments, R₂ is substituted with a halogen. In some embodiments, R₂ is substituted with Cl. In some embodiments, R₂ is substituted with OH. In some embodiments, R₂ is substituted with OH and Cl. In some embodiments, R₂ is

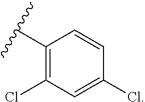 [SMILES: *c1ccc(Cl)cc1Cl]

In some embodiments, R₂ is

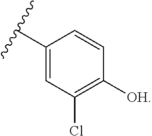 [SMILES: *c1ccc(O)c(Cl)c1]

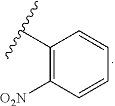 [SMILES: *c1ccccc1[N+](=O)[O-]]

In some embodiments, R₂ is

In some embodiments, formula (I) is

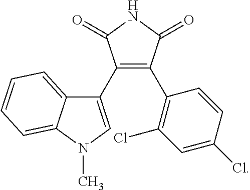 [SMILES: Cn1cc(C2=C(c3ccc(Cl)cc3Cl)C(=O)NC2=O)c2ccccc21]

In some embodiments, formula (I) is

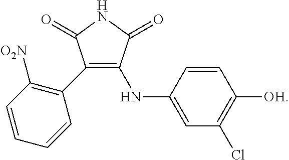 [SMILES: O=C1NC(=O)C(c2ccccc2[N+](=O)[O-])=C1Nc1ccc(O)c(Cl)c1]

As used herein, the terms “alkyl,” “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, adamantly, norbornane, and norbornene. This is also true of groups that include the prefix “alkyl-,” such as alkylcarboxylic acid, alkyl alcohol, alkylcarboxylate, alkylaryl, and the like. Examples of suitable alkylcarboxylic acid groups are methylcarboxylic acid, ethylcarboxylic acid, and the like. Examples of suitable alkylacohols are methylalcohol, ethylalcohol, isopropylalcohol, 2-methylpropan-1-ol, and the like. Examples of suitable alkylcarboxylates are methylcarboxylate, ethylcarboxylate, and the like. Examples of suitable alkyl aryl groups are benzyl, phenylpropyl, and the like.

These may be straight chain or branched, saturated or unsaturated aliphatic hydrocarbon, which may be optionally inserted with N, O, or S. Representative saturated straight chain alkyls include methyl, ethyl, n-propyl, n-butyl, n-pentyl, n-hexyl, and the like; while saturated branched alkyls include isopropyl, sec-butyl, isobutyl, tent-butyl, isopentyl, and the like.

The term “aryl” as used herein includes carbocyclic aromatic rings or ring systems. As used herein, the term “aryl” refers to an aromatic 5-8 membered monocyclic, 8-12 membered bicyclic, or 11-14 membered tricyclic ring system. Examples of aryl groups include phenyl, naphthyl, biphenyl, fluorenyl and indenyl.

The term “heteroaryl” includes aromatic rings or ring systems that contain at least one ring hetero atom (e.g., O, S, N). As used herein, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, thiazolyl, benzimidazolyl, pyrimidinyl, thiophenyl or thienyl, quinolinyl, indolyl, oxazolyl, isoquinolinyl, isoindolyl, thiazolyl, pyrrolyl, tetrazolyl, pyrazolyl, oxazolyl, thiazolyl, benzofuranyl, benzothiophenyl, carbazolyl, benzoxazolyl, benzimidazolyl, quinoxalinyl, benzothiazolyl, naphthyridinyl, isoxazolyl, isothiazolyl, purinyl, quinazolinyl, and the like.

Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5-membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

The aryl, and heteroaryl groups can be unsubstituted or substituted by one or more substituents independently selected from the group consisting of alkyl, alkoxy, methylenedioxy, ethylenedioxy, alkylthio, haloalkyl, haoalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonythio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, arylcarbonylaminoalkyl, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted,” then those groups can also be substituted by one or more of the above enumerated substituents.

As used herein, the term “halogen” refers to iodine, bromine, chlorine, and fluorine.

As used herein, the terms “optionally substituted alkyl,” “optionally substituted cyclyl,” “optionally substituted heterocyclyl,” “optionally substituted aryl,” and “optionally substituted heteroaryl” means that, when substituted, at least one hydrogen atom in said alkyl, cyclyl, heterocylcyl, aryl, or heteroaryl is replaced with a substituent. In the case of an oxo substituent (═O) two hydrogen atoms are replaced. In this regard, substituents include oxo, halogen, alkyl, cyclyl, heterocyclyl, aryl, heteroaryl, —CN, —OR^{x}, —NR^{x}R^{y}, —NR^{x}C(═O)R^{Y}, —NR^{x}SO₂R^{y}, —C(═O)R^{x}, —C(═O)OR^{x}, —C(═O)NR^{x}R^{y}, SOₙR^{x} and —SOₙNR^{x}R^{y}, wherein n is 0, 1 or 2, R^{x} and R^{y} are the same or different and independently hydrogen, alkyl, cyclyl, heterocyclyl, aryl or heterocycle, and each of said alkyl, cyclyl, heterocyclyl, aryl and heterocycle substituents may be further substituted with one or more of oxo, halogen, —OH, —CN, alkyl, —OR^{x}, heterocycle, —NR^{x}R^{y}, —NR^{x}C(═O)R^{y}, —NR^{x}SO₂R^{Y}, —C(═O)R^{x}, —C(═O)OR^{x}, —C(═O)NR^{x}R^{y}, —SOₙR^{x} and —SOₙNR^{x}R^{y}.

As used herein, the term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl” must still comprise the phenyl moiety and cannot be modified by substitution, in this definition, to become, e.g., a heteroaryl group such as pyridine. In a broad aspect, the permissible substituents include acyclic and cyclic, branched and unbranched, carbocyclic and heterocyclic, aromatic and nonaromatic, fused, and bridged substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

In some embodiments, a compound of Formula I can be SB216763 (e.g. a compound having the structure of Fomula II).

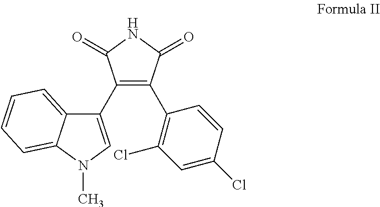 [SMILES: Cn1cc(C2=C(c3ccc(Cl)cc3Cl)C(=O)NC2=O)c2ccccc21]

In some embodiments, a compound of Formula I can be SAB415286 (e.g. a compound having the structure of Formula III).

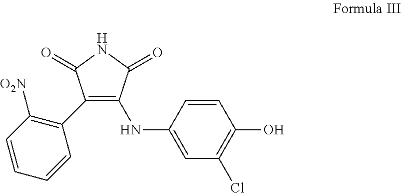 [SMILES: O=C1NC(=O)C(c2ccccc2[N+](=O)[O-])=C1Nc1ccc(O)c(Cl)c1]

The synthesis of SB216763 and SAB415286, as well as the structures and synthesis of related compounds are described, e.g. in WO2000/21927, which is incorporated by reference herein in its entirety. In some embodiments, the method described herein relates to administering a compound described in WO2000/021927.

As used herein, “cardiomyopathy” refers to a disease of the myocardium associated with ventricular dysfunction as defined by the World Health Organization. Dilated cardiomyopathy is characterized by dilatation and impaired contractility of the left (or right) ventricle. Presentation is usually with heart failure. Arrhythmia, thromboembolism, and sudden death are common Hypertrophic cardiomyopathy is characterized by left (or right) ventricular hypertrophy, which is usually asymmetric and involves the interventricular septum. Typically, left ventricular volume is reduced. Systolic gradients are sometimes present. Typical presentations include dyspnea, arrhythmia, and sudden death. Restrictive cardiomyopathy is characterized by restrictive filling of the left (or right) ventricle with normal or near normal ventricular contractility and wall thickness. Presentations are usually with heart failure. The cardiomyopathies are not the only causes of the heart failure syndrome. In western countries, coronary artery disease with resultant ischemic cardiomyopathy remains the primary cause of the heart failure syndrome.

Non-limiting examples of cardiomyopathy can include arrhythmia; arrhythmogenic cardiomyopathy (AC); ischemic heart disease; heart failure; Naxos disease (ARVC), sarcoidosis and giant cell myocarditis. As used herein, “arrhythmia” refers to any of a group of conditions in which there is abnormal electrical activity in the heart. This can cause the heart beat to be too fast (tachycardia) or too slow (bradycardia). Arrhythmia can affect the atria and/or the ventricles and occur at any age. As used herein, “Naxos disease” or “arrhythmogenic right ventricular cardiomyopathy (ARVC)” refers to an inherited heart disease characterized by defects in the surface of heart muscle cells where they are interlinked. Naxos disease is a nonischemic cardiomyopathy that primarily involves the right ventricle and arrhythmia. As used herein, “sarcoidosis” refers to a condiditon in which granulomas form nodules in an organ. Cardiac sarcoiosis can cause conduction abnormalities and ventricular arrhythmia. As used herein, “giant cell myocarditis” refers to a condition which can present with heart failure, ventricular arrhythmia and/or heart block. While the cause is not currentl well understood, heart biopsies indicate the presence of multinucleate giant cells in myocardial tissue.

In some embodiments, the methods described herein relate to treating a subject having or diagnosed as having cardiomyopathy with compound described herein. Subjects having cardiomyopathy can be identified by a physician using current methods of diagnosing cardiomyopathy. Symptoms and/or complications of cardiomyopathy which characterize these conditions and aid in diagnosis are well known in the art and include but are not limited to, dyspnea, arrhythmia, chest pain, embolization, and peripheral edema. Tests that may aid in a diagnosis of, e.g. cardiomyopathy include, but are not limited to, EKG, echocardiogram, and chest radiographs. A family history of cardiomyopathy, or exposure to risk factors for cardiomyopathy can also aid in determining if a subject is likely to have cardiomyopathy or in making a diagnosis of cardiomyopathy.

The compositions and methods described herein can be administered to a subject having or diagnosed as having cardiomyopathy. In some embodiments, the methods described herein comprise administering an effective amount of compositions described herein, e.g. to a subject in order to alleviate a symptom of a cardiomyopathy. As used herein, “alleviating a symptom of a cardiomyopathy” is ameliorating any condition or symptom associated with the cardiomyopathy. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection or topical administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular anti-cardiomyopathic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for arrhythmia among others. The dosage can be determined by a physician and adjusted, as necessary, to suit observed effects of the treatment.

In some embodiments, the technology described herein relates to a pharmaceutical composition as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a compoun as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of compounds as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include pacemakers, defibrillators, ventricular assist devices, ablation, or administration of, e.g. ACE inhibitors (e.g. enalapril, lisinopril, ramipril and captopril); ARBs (e.g. losartan and valsartan); beta blockers (e.g. carvedilol and metoprolol); digoxin (Lanoxin™); and/or diuretics (e.g. bumetanide, furosemide, and spironolactone).

In certain embodiments, an effective dose of a composition can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. arrhythmia by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the compound. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of the compounds described herein, according to the methods described herein depend upon, for example, the form of the compound, its potency, and the extent to which symptoms, markers, or indicators of a condition described herein are desired to be reduced, for example the percentage reduction desired for arrhythmia. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a compound in, e.g. the treatment of a condition described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. electrical activity in the heart. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. heart activity). It is well within the ability of one skilled in the art to monitor efficacy of administration and/or treatment by measuring any one of such parameters, or any combination of parameters. Efficacy can be assessed in animal models of a condition described herein, for example treatment of AC. When using an experimental animal model, efficacy of treatment is evidenced when a statistically significant change in a marker is observed, e.g. arrhythmia or localization of SAP97.

In vitro and animal model assays are provided herein which allow the assessment of a given dose of a compound described herein. By way of non-limiting example, the effects of a dose can be assessed by measuring characteristics of cellular electrophysiology. A non-limiting example of a protocol for such an assay is as follows: zebrafish ventricular myocytes are subjected to current clamp experiments using micropipettes with a tip resistance of 3-5 MΩ. After opening the gigaseal, the microelectrode amplifier (HEKA, EP-10, Lambrecht, Germany) is set to zero holding current and cells are stimulated at a frequency of 0.5 to 1 Hz. Recordings are corrected for a junction potential of 12 mV.^{12,13} For example, I_{Na} and I_{Na} activation and inactivation can be measured by whole-cell voltage-clamp. Cells are held at a membrane potential of −80 mV after which pulses between −120 and +50 mV are applied in 5 mV steps each with a duration of 250 ms. Inactivation is measured from membrane potentials between −120 mV and +50 mV at maximal activation. In view of the small cell size (cell capacitance of 6.7±2.6 pF; n=47) it is not necessary to reduce extracellular [Na⁺].

The efficacy of a given dosage combination can also be assessed in an animal model, e.g. the transgenic zebrafish model of AC described herein. For example, homozygous zebrafish expressing a mutant allele of plakoglobin in the heart can be administered a compound and, e.g. survival and/or the development of arrhythmia measured.

As described herein, the inventors have identified that the level and localization of SAP97 in cardiomyocytes can indicate whether a subject has, or is risk of developing, a cardiomyopathy. In some embodiments, the cardiomyopathy is arrhythmogenic cardiomyopathy.

Accordingly, some embodiments of the invention are generally related to assays, methods and systems for the diagnosis and treatment of cardiomyopathy. In certain embodiments, the assays, methods and systems are directed to determination of the level of SAP97 in a cardiomyocyte. In some embodiments, the assays, methods and systems are directed to determination of the level of SAP97 polypeptide located in the membrane of a cardiomyocyte. In some embodiments, the assays, methods and systems are directed to determination of the level of SAP97 polypeptide located at cell-cell junctions in the membrane of a cardiomyocyte. Treatments for cardiomyopathy are described elsewhere herein. In some embodiments, the treatment can comprise administering a compound of Formula I as described herein. In some embodiments, the treatment can comprise administering SB216763 or SAB415286.

In some embodiments, the methods and assays described herein include (a) transforming the gene target into a detectable gene target; (b) measuring the amount of the detectable gene target; and (c) comparing the amount of the detectable gene target to an amount of a reference, wherein if the amount of the detectable gene target is statistically significantly less than the amount of the reference level, the subject is identified as having, or being likely to develop, cardiomyopathy. In some embodiments, if the amount of the detectable gene target is not statistically significantly less than the amount of the reference level, the subject is identified as not having, or not being at risk of developing cardiomyopathy.

In some embodiments, the reference can be a level of expression of SAP97 in a population of subjects who have not been diagnosed as having, or do not have any symptoms of cardiomyopathy, e.g. arrhythmia. In some embodiments, the reference can also be a level of SAP97 in a control sample, a pooled sample of control individuals or a numeric value or range of values based on the same. Preferably, one looks at the presence and/or absence of a statistically significant change.

The level of SAP97 which is lower than a reference level of SAP97 by at least about 10% than the reference amount, at least about 20%, at least about 30%, at least about 40%, at least about 50%, at least about 80%, at least about 90% or more, is indicative that the subject has, or is at risk of developing, cardiomyopathy.

In one aspect, the technology described herein relates to a method of treatment comprising detecting, in a sample obtained from a subject the level of SAP97 in a cardiomyote, and administering a treatment for cardiomyopathy if the level of SAP97 is statistically significantly lower than a reference level

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from heart tissue to isolated cardiomyotes by disrupting the tissue. A biological/chemical transformation can involve at least one enzyme and/or a chemical reagent in a reaction. For example, a polypeptide can be bound to an antibody reagent.

Methods to measure gene expression products associated with the marker genes described herein are well known to a skilled artisan. Such methods to measure gene expression products, e.g., protein level, include ELISA (enzyme linked immunosorbent assay), western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies for the SAP97 polypeptide are commercially available and can be used for the purposes of the invention to measure protein expression levels, e.g. anti-SAP97 (Cat. No. ab134156; Abcam; Cambridge, Mass.) Alternatively, since the amino acid sequences for SAP97 are known and publically available at NCBI website, one of skill in the art can raise their own antibodies against these proteins of interest for the purpose of the invention.

The amino acid sequences of SAP97 have been assigned NCBI accession numbers for different species such as human, mouse and rat. In particular, the NCBI accession numbers for the amino acid sequences of the human SAP97 (NCBI Gene ID: 1739) include, e.g. NCBI Ref Seq: NP_{—}004078; SEQ ID NO: 1.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (i.e. SAP97 polypeptide as described herein) can also be performed. A known amount of sample and/or antigen is immobilized on a solid support (usually a polystyrene micro titer plate).

Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., cardiomyocyte obtained from a subject) is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5″-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tumor samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

The detection of the level of SAP97 which exists specifically in the membrane (and/or at cell-cell junctions in the membrane) can be determined using methods well known in the art. By way of non-limiting example, immunohistochemistry can be used, and the level of signal from a detectable anti-SAP97 antibody which is localized to the cell membrane can be determined. Histological examination of the results, and/or colocalization with antibody reagents specific for the cell membrane can indicate if a signal originates from the membrane. Alternatively, the membrane fraction of cardiomyocytes can be isolated, e.g. by centrifugation by methods well known in the art. Alternatively, an antibody specific for the extracellular domain of SAP97 can be used to specifically detect SAP97 in the membrane of intact cells (e.g. in a FACS assay), as antibodies will not readily transit the membrane.

In certain embodiments, the gene expression products as described herein can be instead determined by determining the level of messenger RNA (mRNA) expression of genes associated with the marker gene described herein (e.g. SAP97). Such molecules can be isolated, derived, or amplified from a biological sample, such as a tumor biopsy. Detection of mRNA expression is known by persons skilled in the art, and comprise, for example but not limited to, PCR procedures, RT-PCR, Northern blot analysis, differential gene expression, RNA protection assay, microarray analysis, hybridization methods etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

The nucleic acid sequences of SAP97 have been assigned NCBI accession numbers for different species such as human, mouse and rat. For example, the NCBI accession number for a nuclei acid sequence of the human SAP97 is NCBI Ref Seq: NM_{—}004087 (SEQ ID NO: 2). Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, one or more of the reagents (e.g. an antibody reagent) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to 3H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

The term “sample” or “test sample” as used herein denotes a sample taken or isolated from a biological organism, e.g., a tumor sample from a subject. Exemplary biological samples include, but are not limited to, a cardiomyocyte and/or tissue sample etc. The term also includes a mixture of the above-mentioned samples. The term “test sample” also includes untreated or pretreated (or pre-processed) biological samples. In some embodiments, a test sample can comprise cells from subject.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject.

In some aspects, the invention described herein is directed to systems (and computer readable media for causing computer systems) for obtaining data from at least one sample obtained from at least one subject, the system comprising 1) a measuring module configured to measure the level of SAP97 in a cardiomyocyte, 2) a storage module configured to store output data from the measuring module, 3) a comparison module adapted to compare the data stored on the storage module with a reference level, and to provide a retrieved content, and 4) a display module for displaying whether the level of SAP97 is less, by a statistically significant amount, than the reference level and/or displaying the relative levels.

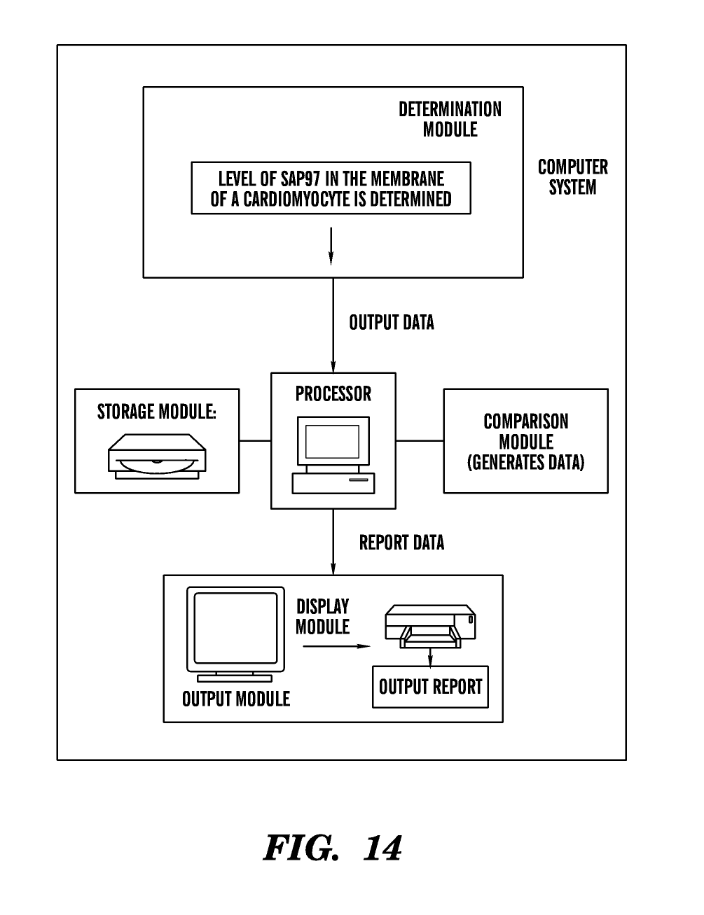

In one embodiment, provided herein is a system comprising: (a) at least one memory containing at least one computer program adapted to control the operation of the computer system to implement a method that includes 1) a measuring module configured to measure the level of SAP97 in a cardiomyocyte in a test sample obtained from a subject, 2) a storage module configured to store output data from the measuring module, 3) a computing module adapted to identify from the output data whether the level in a sample obtained from a subject is statistically significantly less than a reference level, and 4) a display module for displaying a content based in part on the data output from the measuring module, wherein the content comprises a signal indicative of the level of SAP97 and (b) at least one processor for executing the computer program (see FIG. 14).

In some embodiments, the measuring module can measure the presence and/or intensity of a detectable signal from an immunoassay indicating the level of SAP97 polypeptide. Exemplary embodiments of a measuring module can include a FACS machine, automated immunoassay, etc.

The measuring module can comprise any system for detecting a signal elicited from an assay to determine the level of SAP97 as described above herein.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer.

Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of platelet-adherent leukocytes etc. in computer readable form.

The information determined in the measuring system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the measuring module. In additional embodiments, the storage module stores reference information such as levels of SAP97 in the cardiomyocytes of healthy subjects.

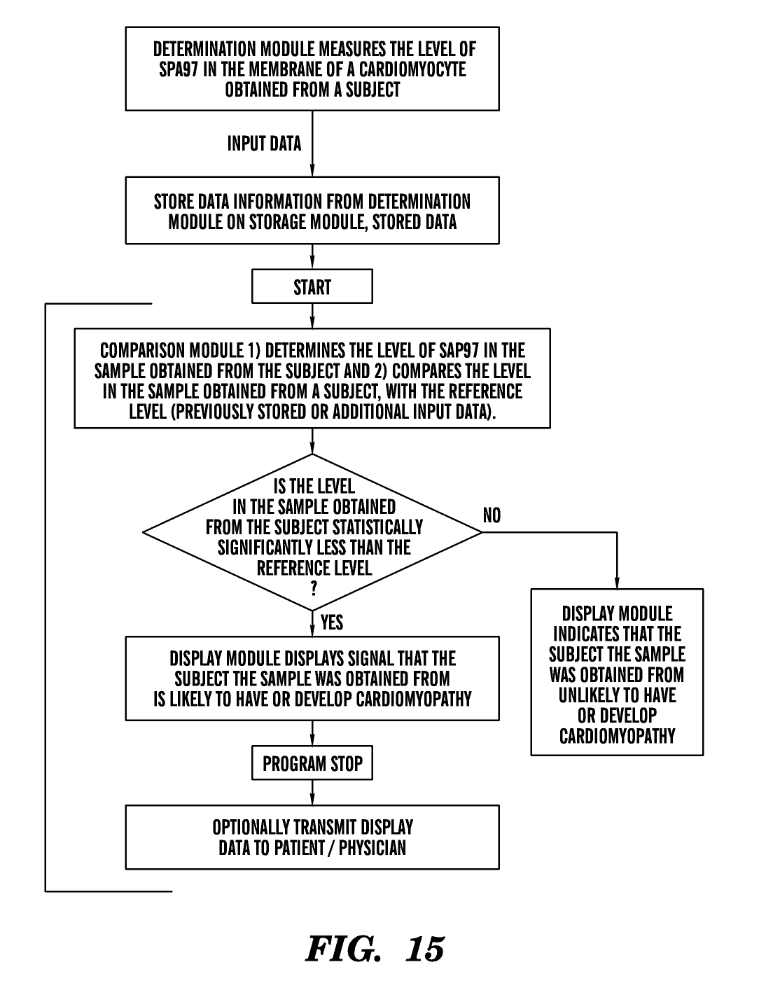

The “computing module” can use a variety of available software programs and formats for computing the level of expression products. Such algorithms are well established in the art. A skilled artisan is readily able to determine the appropriate algorithms based on the size and quality of the sample and type of data. The data analysis tools and equations described herein can be implemented in the computing module of the invention. In some embodiments, the computing module can comprise a computer and/or a computer system. In one embodiment, the computing module further comprises a comparison module, which compares the level of expression products in a sample obtained from a subject as described herein with a reference level as described herein (see, e.g. FIG. 15). By way of an example, when the level of SAP97 in a cardiomyocyte obtained from a subject is measured, a comparison module can compare or match the output data with the mean level of SAP97 in the cardiomyocytes of a population of subjects not having signs or symptoms of cardiomyopathy (i.e. a reference level). In certain embodiments, the mean level of SAP97 in a population of reference subjects can be pre-stored in the storage module. During the comparison or matching process, the comparison module can determine whether the level in a sample obtained from a subject is statistically significantly less than the reference level. In various embodiments, the comparison module can be configured using existing commercially-available or freely-available software for comparison purpose, and may be optimized for particular data comparisons that are conducted.

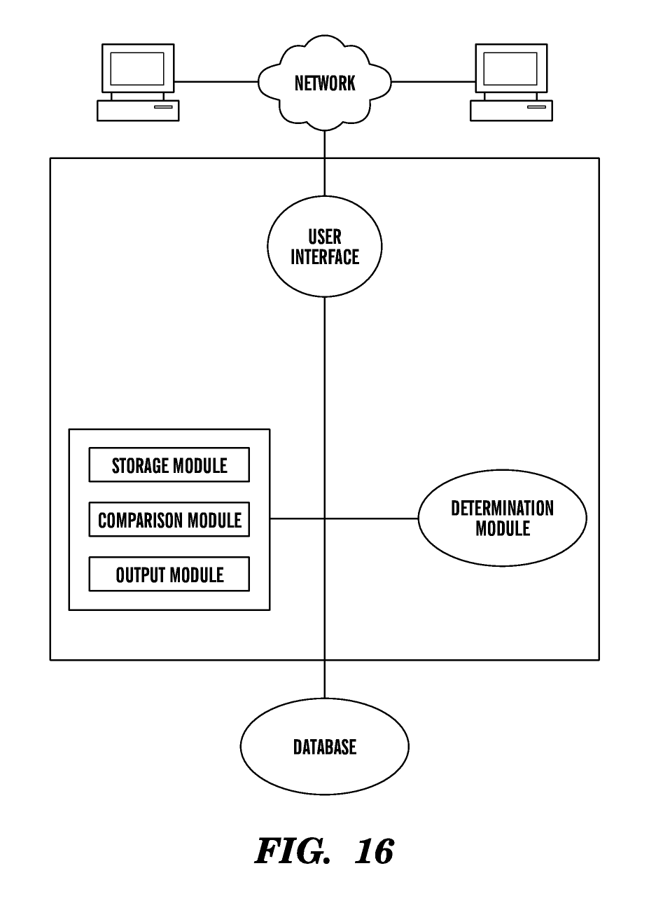

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 16).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be a report, e.g. the level of SAP97 in the cardiomyocyte. In some embodiments, the report can denote raw values of the level of SAP97 or it indicates a percentage or fold increase as compared to a reference level, and/or provides a signal that the subject is or is not likely to have or develop cardiomyopathy as described above herein.

In some embodiments, if the computing module determines that the level of SAP97 in a cardiomyocyte obtained from a subject is less, by a statistically significant amount, than the reference level, the display module provides a report displaying a signal indicating that the level in the sample obtained from a subject is less than that of the reference level. In some embodiments, the content displayed on the display module or report can be the relative level of SAP97 in the sample obtained from a subject as compared to the reference level. In some embodiments, the signal can indicate the degree to which the level of SAP97 in the sample obtained from the subject varies from the reference level. In some embodiments, the signal can indicate that the subject is likely or not likely to have or develop cardiomyopathy. In some embodiments, the content displayed on the display module or report can be a numerical value indicating one of these risks or probabilities. In such embodiments, the probability can be expressed in percentages or a fraction. In some embodiments, the content displayed on the display module or report can be single word or phrases to qualitatively indicate a risk or probability. For example, a word “unlikely” can be used to indicate a lower likelihood of having or developing cardiomyopathy, while “likely” can be used to indicate a high likelihood of having or developing cardiomyopathy.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for determining the level of SAP97, and therefore are not intended to limit the scope of the invention. Variations of the systems and computer readable media described herein are possible and are intended to fall within the scope of the invention. The modules of the machine, or those used in the computer readable medium, may assume numerous configurations. For example, function may be provided on a single machine or distributed over multiple machines.

In one aspect, described herein is a method of determining if a compound can treat a cardiomyopathy, the method comprising contacting a cell with a candidate compound and measuring a cellular response wherein a measurable response indicates the candidate compound can treat a cardiomyopathy and wherein the cellular response is selected from the group consisting of: gap junction remodeling; prevention of redistribution of plakoglobin from junctional to intracellular pools; decreased myocyte apoptosis; increased levels of SAP97; normalization of the nppb response; a decrease in the nuclear accumulation of plakoglobin; normalization of Cx43 levels; normalization of PMP and dV/dtₘₐₓ; and normalization of I_{Na} or I_{Ki} current densities. These cellular responses and methods of measuring them are known to one of ordinary skill in the art and are described elsewhere herein.

In some embodiments, the candidate compound is a compound of Formula I as described above herein. In some embodiments, the compound of Formula I is selected from the group consisting of SB216763 and SAB415286.

In some embodiments, the cell is a cardiomyocyte. In some embodiments, the cell is obtained from a subject with a cardiomyopathy, e.g. a cardiomyocyte, skin cell, and/or peripheral blood cell. Testing the response of a cell obtained from a particular subject can determine if a given compound will be efficacious in treatment of that same subject. In some embodiments, the cell is an iPS cell derived from a subject. In some embodiments, the cell is a cardiomyocyte obtained by differentiating an iPS cell derived from a subject.

In some embodiments, the cardiomyopathy is selected from the group consisting of arrhythmia; arrhythmogenic cardiomyopathy (AC); ischemic heart disease; heart failure; Naxos disease (ARVC); sarcoidosis; and giant cell myocarditis.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal The mammal can be a human, non-human primate, mouse, rat, dog, cat, horse, or cow, but is not limited to these examples. Mammals other than humans can be advantageously used as subjects that represent animal models of cardiomyopathy. A subject can be male or female.

A subject can be one who has been previously diagnosed with or identified as suffering from or having a condition in need of treatment (e.g. cardiomyopathy) or one or more complications related to such a condition, and optionally, have already undergone treatment for cardiomyopathy or the one or more complications related to cardiomyopathy. Alternatively, a subject can also be one who has not been previously diagnosed as having cardiomyopathy or one or more complications related to cardiomyopathy. For example, a subject can be one who exhibits one or more risk factors for cardiomyopathy or one or more complications related to cardiomyopathy or a subject who does not exhibit risk factors.

A “subject in need” of treatment for a particular condition can be a subject having that condition, diagnosed as having that condition, or at risk of developing that condition.

As used herein, “SAP97,” “synapse-associated protein 97,” or “DLG1” refers to a transmembrane protein expressed in a number of cells. As described herein, in cardiomyocytes, SAP97 may be involved in the trafficking of proteins to the membrane, e.g. at the cell-cell junction and may influence cell-cell adhesion.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab')₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab')2, Fd fragments, Fv fragments, scFv, and domain antibodies (dAb) fragments (see, e.g. de Wildt et al., Eur J. Immunol 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab')2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

A further kind of antibody reagent is an intrabody i.e. an intracellular antibody (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Intrabodies work within the cell and bind intracellular protein. Intrabodies can include whole antibodies or antibody binding fragments thereof, e.g. single Fv, Fab and F(ab)′2, etc. Methods for intrabody production are well known to those of skill in the art, e.g. as described in WO 2002/086096. Antibodies will usually bind with at least a KD of about 1 mM, more usually at least about 300 μM, typically at least about 10 μM, more typically at least about 30 μM, preferably at least about 10 μM, and more preferably at least about 3 μM or better.).

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives to hybridoma-based production systems. The main advantages of transgenic animals are potential high yields from renewable sources.

As used herein, an “epitope” can be formed both from contiguous amino acids, or noncontiguous amino acids juxtaposed by folding of a protein. Epitopes formed from contiguous amino acids are typically retained on exposure to denaturing solvents, whereas epitopes formed by folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

Nucleic acid molecules encoding amino acid sequence variants of antibodies are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, antigen-binding portion thereof, or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, NY, 1982 and 1989), and Ausubel, 1987, 1993, and can be used to construct nucleic acid sequences which encode a monoclonal antibody molecule or antigen binding region thereof. A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of an antibody or antigen-binding portion thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence. Moreover, problems associated with retention of initiation codon-derived methionine residues in direct yeast (or bacterial) expression may be avoided. Sabin et al., 7 Bio/Technol. 705 (1989); Miller et al., 7 Bio/Technol. 698 (1989). Any of a series of yeast gene expression systems incorporating promoter and termination elements from the actively expressed genes coding for glycolytic enzymes produced in large quantities when yeast are grown in media rich in glucose can be utilized to obtain recombinant antibodies or antigen-binding portions thereof. Known glycolytic genes can also provide very efficient transcriptional control signals. For example, the promoter and terminator signals of the phosphoglycerate kinase gene can be utilized.

Production of antibodies or antigen-binding portions thereof as described herein can be achieved in insects, for example, by infecting the insect host with a baculovirus engineered to express a transmembrane polypeptide by methods known to those of skill in the art. See Ausubel et al., 1987, 1993.

In some embodiments, the introduced nucleotide sequence is incorporated into a plasmid or viral vector capable of autonomous replication in the recipient host. Any of a wide variety of vectors can be employed for this purpose and are known and available to those of ordinary skill in the art. See, e.g., Ausubel et al., 1987, 1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli., for example. Other gene expression elements useful for the expression of cDNA encoding antibodies or antigen-binding portions thereof include, but are not limited to (a) viral transcription promoters and their enhancer elements, such as the SV40 early promoter (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983) Immunoglobulin cDNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., 1 Protein Engin. 499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene is assembled in, or inserted into, an expression vector. Recipient cells capable of expressing the chimeric immunoglobulin chain gene product are then transfected singly with an antibody, antigen-binding portion thereof, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the fused genes encoding the antibody, antigen-binding fragment thereof, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the fused genes in a bacterial system. The genes so produced and amplified in a bacterial host are subsequently used to co-transfect a eukaryotic cell, and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused genes encoding chimeric H and L chains can be assembled on the same expression vector.

For transfection of the expression vectors and production of the chimeric, humanized, or composite human antibodies described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, or antigen-binding portion thereof as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Yeast provides certain advantages over bacteria for the production of immunoglobulin H and L chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled chimeric, humanized, or composite human antibodies, portions and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or peptides described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of chimeric, humanized, or composite humanized antibodies and fragments thereof encoded by the cloned immunoglobulin cDNAs or CDRs in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein.

In some embodiments, one or more antibodies or antibody reagents thereof as described herein can be produced in vivo in an animal that has been engineered or transfected with one or more nucleic acid molecules encoding the polypeptides, according to any suitable method.

In some embodiments, an antibody or antibody reagent as described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Many vector systems are available for the expression of cloned H and L chain genes in mammalian cells (see Glover, 1985). Different approaches can be followed to obtain complete H₂L₂ antibodies. As discussed above, it is possible to co-express H and L chains in the same cells to achieve intracellular association and linkage of H and L chains into complete tetrameric H₂L₂ antibodies or antigen-binding portions thereof. The co-expression can occur by using either the same or different plasmids in the same host. Genes for both H and L chains or portions thereof can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antibodies, antigen-binding portions thereof and/or H₂L₂ molecules via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

Additionally, plants have emerged as a convenient, safe and economical alternative main-stream expression systems for recombinant antibody production, which are based on large scale culture of microbes or animal cells. Antibodies can be expressed in plant cell culture, or plants grown conventionally. The expression in plants may be systemic, limited to susb-cellular plastids, or limited to seeds (endosperms). See, e.g., U.S. Patent Pub. No. 2003/0167531; U.S. Pat. No. 6,080,560; U.S. Pat. No. 6,512,162; WO 0129242. Several plant-derived antibodies have reached advanced stages of development, including clinical trials (see, e.g., Biolex, NC).

In some aspects, provided herein are methods and systems for the production of a humanized antibody, which is prepared by a process which comprises maintaining a host transformed with a first expression vector which encodes the light chain of the humanized antibody and with a second expression vector which encodes the heavy chain of the humanized antibody under such conditions that each chain is expressed and isolating the humanized antibody formed by assembly of the thus-expressed chains. The first and second expression vectors can be the same vector. Also provided herein are DNA sequences encoding the light chain or the heavy chain of the humanized antibody; an expression vector which incorporates a said DNA sequence; and a host transformed with a said expression vector.

Generating a humanized antibody from the sequences and information provided herein can be practiced by those of ordinary skill in the art without undue experimentation. In one approach, there are four general steps employed to humanize a monoclonal antibody, see, e.g., U.S. Pat. No. 5,585,089; U.S. Pat. No. 6,835,823; U.S. Pat. No. 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties) by splicing genes from a mouse, or other species, antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (see, e.g. Skerra et al., Science 242:1038-1041 (1988); which is incorporated by reference herein in its entirety).

Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. E. coli is one prokaryotic host particularly useful for cloning the DNA sequences. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization. Mammalian cells are a preferred host for expressing nucleotide segments encoding immunoglobulins or fragments thereof. See Winnacker, From Genes to Clones, (VCH Publishers, NY, 1987), which is incorporated herein by reference in its entirety. A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., “Cell-type Specific Regulation of a Kappa Immunoglobulin Gene by Promoter and Enhancer Elements,” Immunol Rev 89:49 (1986), incorporated herein by reference in its entirety), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., “Chimeric and Humanized Antibodies with Specificity for the CD33 Antigen,” J Immunol 148:1149 (1992), which is incorporated herein by reference in its entirety. Alternatively, antibody coding sequences can be incorporated in transgenes for introduction into the genome of a transgenic animal and subsequent expression in the milk of the transgenic animal (e.g., according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin. The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra, which is herein incorporated by reference in is entirety). For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982), which is incorporated herein by reference in its entirety).

Once expressed, the whole antibodies, their dimers, individual light and heavy chains, or other immunoglobulin forms of the present invention can be recovered and purified by known techniques, e g , immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, and the like. See generally, Vols. I & II Immunol. Meth. (Lefkovits & Pernis, eds., Acad. Press, NY, 1979 and 1981).

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a cancer cell marker.

As used herein, the terms “treat,” “treatment” “treating,” or “amelioration” refer to therapeutic treatments, wherein the object is to reverse, alleviate, ameliorate, inhibit, slow down or stop the progression or severity of a condition associated with a disease or disorder, e.g. cardiomyopathy. The term “treating” includes reducing or alleviating at least one adverse effect or symptom of a condition, disease or disorder associated with a cardiomyopathy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of refers to compositions, methods, and respective components thereof as described herein, which are exclusive of any element not recited in that description of the embodiment.

As used herein the term “consisting essentially of refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating cardiomyopathy, the method comprising administering a compound of

Formula I:

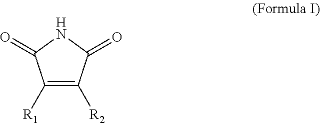

wherein R₁ is optionally substituted heteroaromatic or —NR₂R₃; R₂ is optionally substituted aryl; and R₃ is H or alkyl.
2. The method of paragraph 1, wherein the cardiomyopathy is selected from the group consisting of:
   arrhythmia; arrhythmogenic cardiomyopathy (AC); ischemic heart disease; heart failure;
   Naxos disease (ARVC); sarcoidosis; and giant cell myocarditis.
3. The method of paragraph 2, wherein the cardiomyopathy is arrhythmogenic cardiomyopathy (AC).
4. The method of any of paragraphs 1-3, wherein the compound of Formula I is selected from the group consisting of:
   SB216763 and SAB415286.
5. An assay comprising:
   measuring the level of SAP97 polypeptide in a test sample obtained from a subject;
   wherein a decrease in the SAP97 polypeptide level relative to a reference level indicates the subject has a higher risk of having or developing cardiomyopathy.
6. An assay comprising:
   contacting a sample obtained from the subject with a SAP97-specific antibody reagent to detect the presence or level of a SAP97 polypeptide;
   measuring the presence or intensity of a signal which indicates the presence or level of SAP97 polypeptide in the sample;
   wherein a decrease in the SAP97 polypeptide level relative to a reference level indicates the subject has a higher risk of having or developing cardiomyopathy.
7. An assay for selecting a treatment regimen for a subject with cardiomyopathy, comprising:
   measuring the level of SAP97 polypeptide in a test sample obtained from a subject;
   selecting a treatment regimen comprising administering a compound of Formula I:

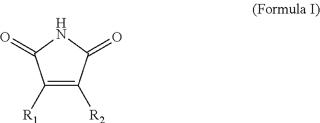

wherein R₁ is optionally substituted heteroaromatic or —NR₂R₃; R₂ is optionally substituted aryl; and R₃ is H or alkyl, when a decreased level of SAP97 polypeptide, relative to a reference level, is measured.
8. A method of identifying a subject in need of treatment for cardiomyopathy, the method comprising:
   measuring/detecting the level of SAP97 polypeptide in a test sample obtained from a subject; and
   identifying the subject as being in need of treatment for cardiomyopathy when the level of SAP97 polypeptide is decreased relative to a reference level.
9. A method of determining if a subject is at risk for cardiomyopathy, the method comprising:
   providing a sample obtained from the subject;
   measuring the level of SAP97 polypeptide in a test sample obtained from a subject;
   comparing the level of SAP97 polypeptide in the sample to a reference level of SAP97 polypeptide;
   determining that the subject is at risk for cardiomyopathy when the level of SAP97 polypeptide is decreased relative to a reference level; and
   determining that the subject is not at risk for cardiomyopathy when the level of SAP97 polypeptide is not decreased relative to a reference level.
10. A method of determining the efficacy of a treatment for cardiomyopathy, the method comprising:
(a) measuring/detecting the level of SAP97 polypeptide in a test sample obtained from a subject before administration of the treatment;
(b) measuring/detecting the level of SAP97 polypeptide in a test sample obtained from a subject after administration of the treatment; and
(c) determining that the treatment is not efficacious when the expression level determined in step (b) is decreased relative to the expression level determined in step (a); and
(d) determining that the treatment is efficacious when the expression level determined in step (b) is increased relative to the expression level determined in step (a).
11. A method of treatment for cardiomyopathy comprising;
measuring/detecting the level of SAP97 polypeptide in a test sample obtained from a subject;
treating the subject with a compound of Formula I:

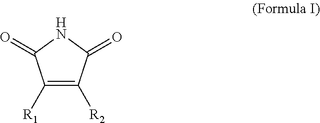

wherein R₁ is optionally substituted heteroaromatic or —NR₂R₃; R₂ is optionally substituted aryl; and R₃ is H or alkyl, -o1 when the level of SAP97 polypeptide is decreased relative to a reference level.
12. A method of treating cardiomyopathy comprising;
administering a therapeutically effective amount of a compound of Formula I:

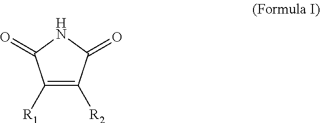

wherein R₁ is optionally substituted heteroaromatic or —R₂R₃; R₂ is optionally substituted aryl; and R₃ is H or alkyl,
to a subject determined to be in need of treatment for cardiomyopathy and further determined to have a level of SAP97 that is decreased relative to a reference level.
13. The assay or method of any of paragraphs 5-12, wherein the test sample comprises a cardiomyocyte.
14. The assay or method of any of paragraphs 5-13, wherein the level of a SAP97 polypeptide is the level of SAP97 polypeptide in a cardiomyocyte.
15. The assay or method of paragraph 14, wherein the level of SAP97 polypeptide present in the cardiomyocyte is the level of SAP97 polypeptide located in the membrane of a cardiomyocyte.
16. The assay or method of calim 15, wherein the level of SAP97 polypeptide present in the membrane is the level of SAP97 polypeptide located at cell-cell junctions.
17. The assay or method of any of paragraphs 5-16, wherein the treatment comprises administering a composition of Formula I:

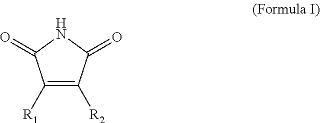

wherein R₁ is optionally substituted heteroaromatic or —NR₂R₃; R₂ is optionally substituted aryl; and R₃ is H or alkyl.
18. The assay or method of paragraph 17, wherein the treatment comprises administering SB216763 or SAB415286.
19. The assay or method any of paragraphs 5-18, wherein the cardiomyopathy is selected from the group consisting of:
arrhythmia; arrhythmogenic cardiomyopathy (AC); ischemic heart disease; heart failure;
Naxos disease (ARVC); sarcoidosis; and giant cell myocarditis.
20. The assay or method of paragraph 19, wherein the cardiomyopathy is arrhythmogenic cardiomyopathy (AC).
21. The assay or method of any of paragraphs 5-20, wherein a detectable signal is generated by a SAP97-specific antibody reagent when a SAP97 polypeptide is present.
22. The assay or method of paragraph 21, wherein the antibody reagent is detectably labeled or capable of generating a detectable signal.
23. The assay or method of any of paragraphs 5-22, wherein the level of the polypeptide is determined using a method selected from the group consisting of:
Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy; FACS; and immunoelectrophoresis assay.
24. The assay or method of any of paragraphs 5-23, wherein the polypeptide level is measured using immunochemistry.
25. The assay or method of any of paragraphs 5-24, wherein the level of SAP97 polypeptide is normalized relative to the expression level of one or more reference genes or reference proteins.
26. The assay or method of any of paragraphs 5-25, wherein the reference level of SAP97 polypeptide is the level of SAP97 polypeptide in a prior sample obtained from the subject.
27. The assay or method of any of paragraphs 5-26, further comprising a step of obtaining a sample from the subject.
28. A kit comprising a SAP97-specific antibody reagent.
29. A computer system for determining the risk of a subject having or developing cardiomyopathy the system comprising:
a measuring module configured to measure the level of SAP97 polypeptide in a test sample obtained from a subject;
a storage module configured to store output data from the determination module;
a comparison module adapted to compare the data stored on the storage module with a reference level, and to provide a retrieved content, and
a display module for displaying whether the sample comprises a level of SAP97 polypeptide which is significantly decreased relative to the reference expression level and/or displaying the relative level of SAP97 polypeptide.
30. The system of paragraph 29, wherein the measuring module measures the intensity of a detectable signal from an assay indicating the level of SAP97 polypeptide in the test sample.
31. The system of paragraph 30, wherein the assay is an immunoassay.
32. The system of any of paragraphs 29-31, wherein if the computing module determines that the level of SAP97 in the test sample obtained from a subject is lower by a statistically significant amount than the reference level, the display module displays a signal indicating that the levels in the sample obtained from a subject are lower than those of the reference level.
33. The system of any of paragraphs 29-32, wherein the signal indicates that the subject has an increased likelihood of having or developing cardiomyopathy.
34. The system of any of paragraphs 29-33, wherein the signal indicates the subject is in need of treatment for cardiomyopathy.
35. The system of any of paragraphs 29-34, wherein the signal indicates the degree to which the level of SAP97 polypeptide in the sample obtained from a subject varies from the reference level.
36. The system of any of paragraphs 29-35, wherein the test sample comprises a cardiomyocyte.
37. The system of any of paragraphs 29-36, wherein the level of a SAP97 polypeptide is the level of SAP97 polypeptide in a cardiomyocyte.
38. The system of paragraph 37, wherein the level of SAP97 polypeptide present in the cardiomyocyte is the level of SAP97 polypeptide located in the membrane of a cardiomyocyte.
39. The system of paragraph 38, wherein the level of SAP97 polypeptide present in the membrane is the level of SAP97 polypeptide located at cell-cell junctions.
40. A method comprising:
contacting a cell with a candidate compound;
measuring a cellular response;
wherein a measurable response indicates the candidate compound can treat a cardiomyopathy and selecting a compound exhibiting a cellular response; and
wherein the cellular response is selected from the group consisting of:
   gap junction remodeling; prevention of redistribution of plakoglobin from junctional to intracellular pools; decreased myocyte apoptosis; increased levels of SAP97; normalization of the nppb response; a decrease in the nuclear accumulation of plakoglobin; normalization of Cx43 levels; normalization of PMP and dV/dtₘₐₓ; and normalization of I_{Na} or I_{Ki} current densities.
41. The method of paragraph 40, wherein the candidate compound is a compound of Formula I:

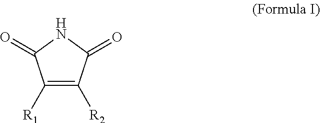

wherein R₁ is optionally substituted heteroaromatic or —NR₂R₃; R₂ is optionally substituted aryl; and R₃ is H or alkyl.
42. The method of paragraph 41, wherein the compound of Formula I is selected from the group consisting of:
SB216763 and SAB415286.
43. The method of any of paragraphs 40-42, wherein the cell is a cardiomyocyte.
44. The method of any of paragraphs 40-43, wherein the cell is obtained from a subject with a cardiomyopathy.
45. The method of any of paragraphs 40-44, wherein the cell is an iPS cell derived from a subject.
46. The method of any of paragraphs 40-45, wherein the cell is a cardiomyocyte obtained by differentiating an iPS cell derived from a subject.
47. The method of any of paragraphs 40-46, wherein the cardiomyopathy is selected from the group consisting of:
arrhythmia; arrhythmogenic cardiomyopathy (AC); ischemic heart disease; heart failure; Naxos disease (ARVC); sarcoidosis; and giant cell myocarditis.
48. An engineered non-human cell expressing 2057del2 plakoglobin.
49. The cell of paragraph 48, wherein the cell is a cardiomyocyte.
50. The cell of any of paragraphs 48-49, wherein the cell is a zebrafish cell.
51. The cell of any of paragraphs 48-49, wherein the cell is a murine cell.
52. A transgenic animal comprising a cell of any of paragraphs 48-51.
53. The animal of paragraph 52, wherein the animal is a zebrafish.
54. The animal of paragraph 52, wherein the animal is a mouse.

### EXAMPLES

### Example 1

### Activation of Wnt Signaling Prevents Pathological Changes Associated with Arrhythmogenic Right Ventricular Cardiomyopathy

Redistribution of the desmosomal protein plakoglobin (y-catenin) from junctional to intracellular pools occurs in the myocardium of patients with arrhythmogenic right ventricular cardiomyopathy (ARVC). Reduced expression of the desmosomal protein desmoplakin in an atrial myocyte cell line also leads to nuclear localization of plakoglobin and a 2-fold reduction in canonical Wnt/β-catenin signaling through Tcf/Lefl transcription factors. It is demonstrated herein that activation of Wnt signaling is sufficient to reverse pathological features of ARVC caused by expression of a mutant desmosomal protein.

Neonatal rat ventricular myocytes were transfected to express a mutant form of plakoglobin (2057del2) known to cause a highly penetrant form of ARVC (Naxos disease). Cells expressing 2057del2 show many features seen in human ARVC including intracellular and nuclear redistribution of junctional plakoglobin, remodeling of gap junctions, secretion of cytokines, and increased myocyte apoptosis. To characterize the effects of altered Wnt signaling in these pathological processes, myocytes expressing mutant plakoglobin were incubated with SAB216763 or SAB415286 (both activators of Wnt signaling), or niclosamide which blocks Wnt signaling by depleting upstream regulators (frizzled and disheveled). The distribution of intercalated disk proteins, rates of apoptosis and cytokine production were investigated in treated cultures.

Increased myocyte apoptosis due to expression of 2057del2 was totally abolished by SAB216763. Gap junction remodeling was also blocked by SAB216763. Both SAB216763 and SAB415286 prevented redistribution of plakoglobin from junctional to intracellular pools while niclosamide increased nuclear localization of plakoglobin. None of the Wnt modulators affected cytokine production in cells expressing 2057del2.

Provided herein is the first demonstration that connects reduced Wnt signaling to key features of disease expression in ARVC. These findings permit new therapeutic strategies in patients with ARVC.

### Example 2

### An ARVC Model for Drug Discovery

The model described herein focuses, for screening purposes, on modeling the Naxos variant for several reasons. This syndrome is one of the most malignant forms of AC, and exhibits some evidence of dose dependence on the mutant allele (a truncation of the plakoglobin gene 2157del2 which leads to loss of the c-terminal 56 amino acids of the final protein)-there are subtle heterozygous as well as the more penetrant homozygous forms. As a result, it is well suited to being modeled precisely in simple transgenic overexpression of the mutant form.

The clmc2 promoter was cloned and homozygous clmc2::GAL4 driver lines generated. Human plakoglobin cDNA was used to generate the truncated mutant cDNA (confirmed by resequencing) cloned downstream multiple copies of the UAS sequence (4 and 10 copies in different lines). This was used to generate transgenic lines that were then bred to homozygosity. An intercross between the two lines generates zebrafish expressing the mutant transgene from approximately ˜16hpf onwards and restricted to the heart.

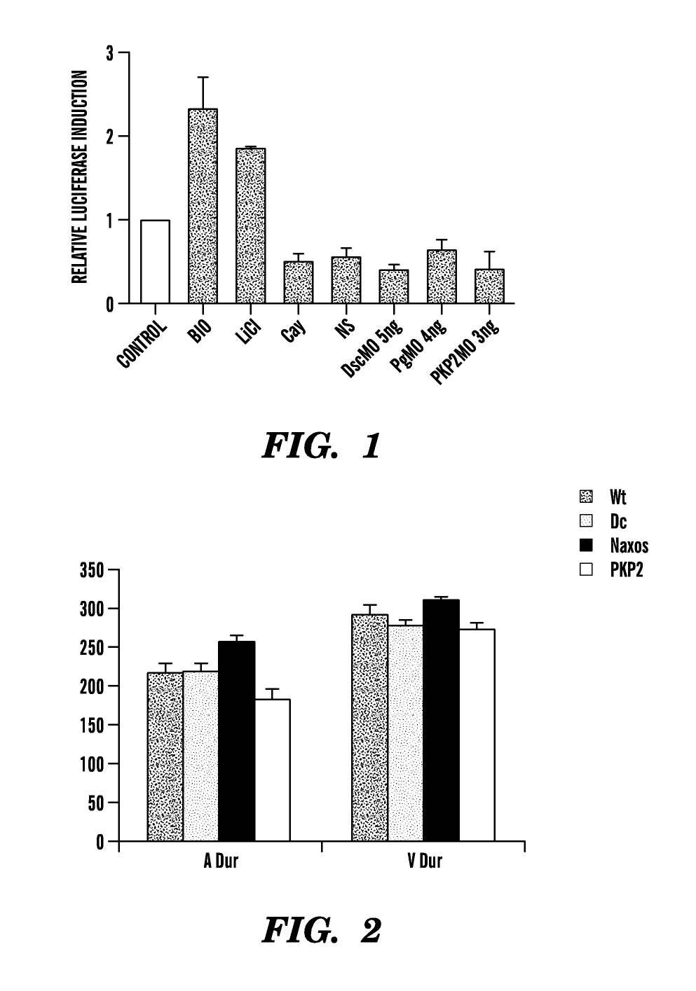

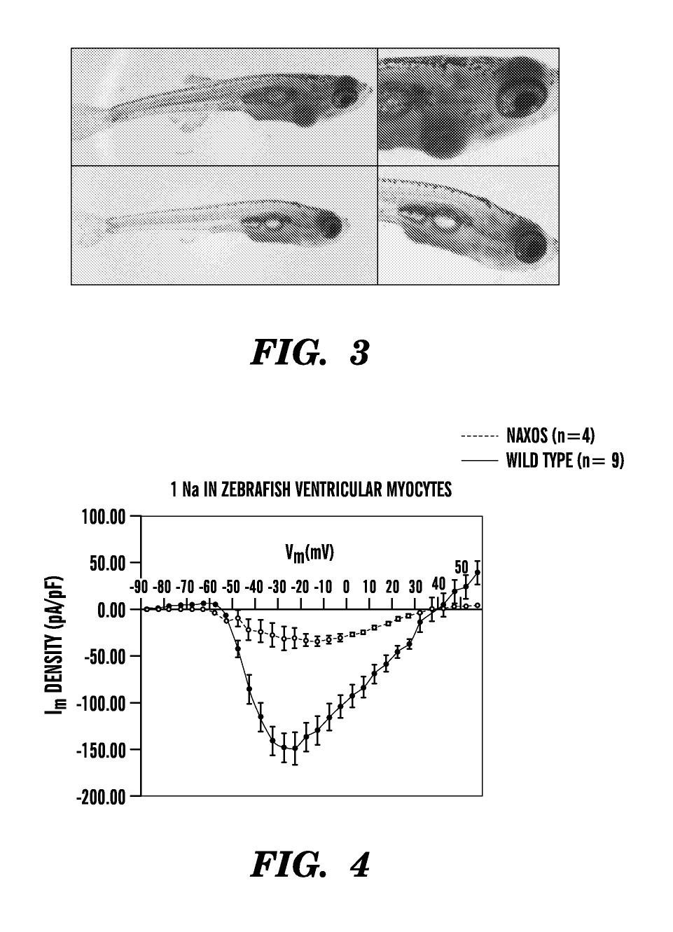

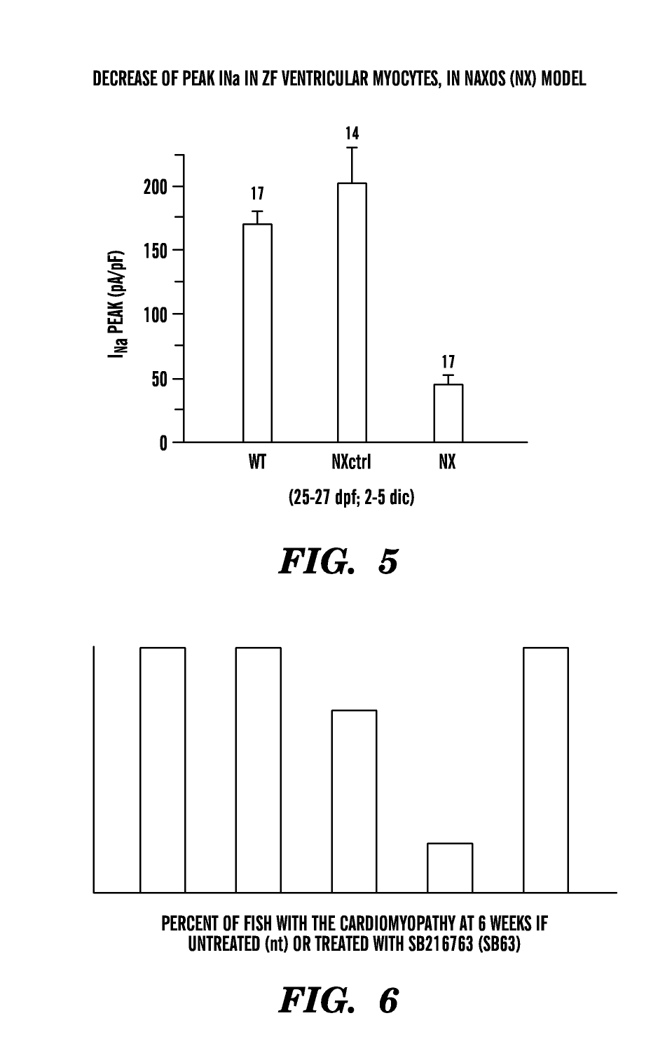

The embryos exhibit no detectable defect in heart rate, contractility, or cardiac output and this lack of an overt phenotype is sustained during the larval stages. Larval stages do exhibit subtle abnormalities of:
1. Gene expression-including inhibition of canonical Wnt signaling and induction of nppb (FIG. 1)
2. Impulse propagation There are no direct data from Naxos patients or other Naxos models on these parameters, but conduction is abnormal from an early age in affected individuals. However, there are data on nppb elevation in other models of ARVC or in human ARVC which are concordant with these data. (FIG. 2)
3. By 4-6 weeks of age there is a very consistent phenotype. Overt cardiomegaly and peripheral edema evident in >80% of animals. (FIG. 3) Abnormal contractility on non-invasive testing. Histological evidence of myocardial loss and fibrosis
4. Electrophysiologic evidence of sodium channel loss at membrane (FIG. 4)
5. Consistently higher mortality observed with almost all mutant fish dying by 6 months (FIG. 5)

Features conserved in the model include:
Contractility and cardiac output defects
Electrophysiology defects
Structural changes in ventricular myocardium
Ultrastructural changes in desmosomes and other junctions
Numerous conserved molecular changes.

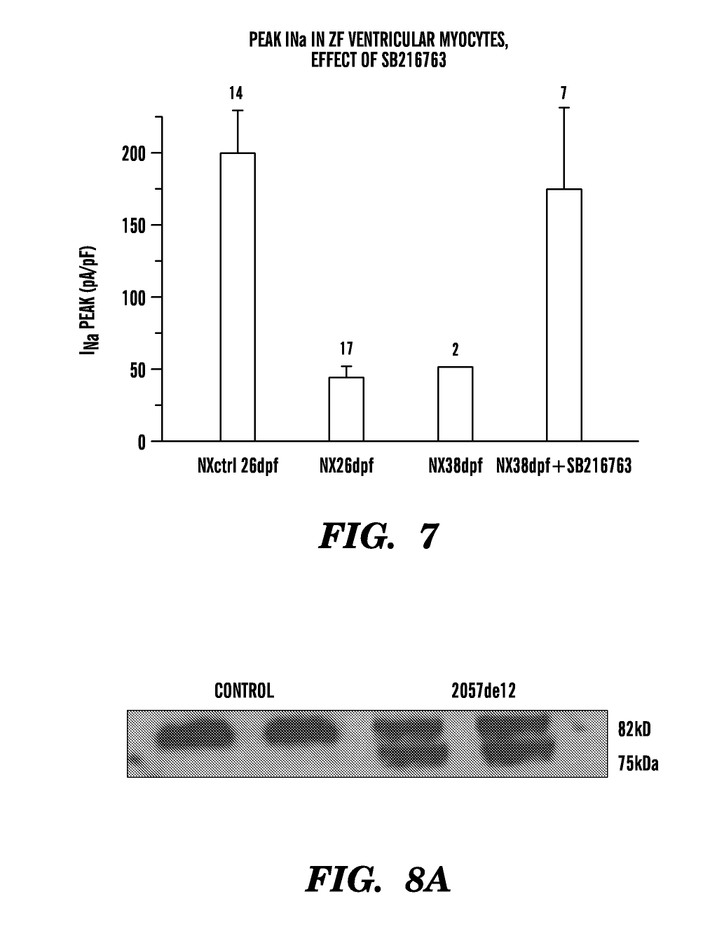

Rescue with SB216763. An initial screen was performed using the original 96 well plate assay as described and compound SB216763 was identified as a normalizer of the nppb response in transgenic reporter fish. (FIGS. 6-7)

### Example 3

Arrhythmogenic cardiomyopathy (AC) is characterized by frequent arrhythmias as an early manifestation of disease in hearts that otherwise appear normal. To elucidate underlying mechanisms and discover new drug therapies, a zebrafish model of AC with cardiac myocyte-specific expression of the human 2057del2 mutation in plakoglobin was studied. As described herein, SB216763, an activator of Wnt signaling, rescues the heart failure phenotype and reduces mortality. Zebrafish ventricular myocytes expressing 2057del2 plakoglobin show marked action potential remodeling related to 70-80% reductions in I_{Na} and I_{Ki} current densities which are rapidly normalized after exposure to SB216763. Neonatal rat ventricular myocytes expressing 2057del2 plakoglobin recapitulate fundamental features of AC in patients including redistribution of plakoglobin from junctional to intracellular sites, gap junction remodeling, cytokine expression and myocyte apoptosis, all of which are normalized by SB216763. The disease phenotype and its rapid reversal by SB216763 are associated with marked changes in subcellular distribution of plakoglobin, connexin43 and Nav1.5 but without changes in total cellular content of these proteins, suggesting a trafficking defect mediated by altered Wnt signaling. In further support of this mechanism, abnormal distribution of the PDZ protein SAP97 was observed in cardiac myocytes expressing 2057del2 plakoglobin and in AC patient samples. Thus, SAP97, known to play a critical role in forward trafficking of Nav1.5 and Kir2.1, is a component of the disease pathway in AC. These observations link myocyte injury and electrophysiological derangements to a common disease mechanism in AC.

Arrhythmogenic cardiomyopathy (AC) is a highly arrhythmogenic form of human heart disease and a significant cause of sudden death in the young.^{1,2} First described as a right ventricular disease (arrhythmogenic right ventricular cardiomyopathy or ARVC), AC is now recognized to include biventricular and left dominant forms which may be misdiagnosed as dilated cardiomyopathy or myocarditis.³ Degeneration of cardiac myocytes and replacement by fibrofatty scar tissue develop with disease progression, but arrhythmias are the cardinal feature of AC. Rhythm disturbances are usually the earliest manifestation of disease and often precede structural remodeling of the myocardium.^{1,2}

Indeed, there is something fundamentally arrhythmogenic about AC, particularly in its early stages in which frequent arrhythmias occur in otherwise apparently normal hearts. In this sense, AC is more reminiscent of the ion channelopathies than the other non-ischemic cardiomyopathies. Understanding arrhythmias in AC can, therefore, provide insights into arrhythmogenesis and permit new strategies for treating sudden death not only in AC but, in other more common forms of heart disease.

The sudden death of an apparently healthy young adult is often the precipitating event identifying a family that carries AC.¹ Patients with demonstrated or strongly suspected risk of sudden death usually receive implantable defibrillators, because there are no mechanism-based therapies to prevent arrhythmias or limit myocardial injury in this disease spectrum. To gain insights into fundamental disease mechanisms in AC and to discover new drug therapies, we the GAL4/UAS transactivation system was used to create a transgenic model of AC in zebrafish with cardiac myocyte-specific expression of the human 2057del2 mutation in the desmosomal protein plakoglobin (y-catenin).⁴ This mutation causes Naxos disease in patients, a highly penetrant form of ARVC associated with changes in hair and skin.^{5,6} Transgenic fish expressing this mutation exhibit bradycardia, and reduced cardiac output by 48 hr post-fertilization, with progression to cardiomegaly, peripheral edema and substantial mortality by the time maturity is reached.⁴ To adapt this line for high-throughput screening, founders were mated with a natriuretic peptide b (nppb)-luciferase reporter line previously validated for use in 96-well plates,' and native nppb expression was strongly correlated with semi-automated luciferase activity.⁴>1,000 chemicals from a library of bioactive compounds were screened for disease modifiers and compounds were identified that either suppressed or exacerbated the disease phenotype. One such suppressor, SB2, an activator of canonical Wnt signaling, reduced nppb expression and bradycardia, improved contractility and significantly increased survival at 28 days.⁴

Described herein is the characterization of the cellular electrophysiology of zebrafish cardiac myocytes expressing 2057del2 plakoglobin and demonstration that action potential remodeling is related to marked reductions in the densities of I_{Na} and k_{K1}, both of which are rapidly normalized following brief exposure to SB216763. Also described herein is the development of a mammalian cardiac myocyte model of AC that recapitulates fundamental features of the disease seen in patients, and these features are also normalized by SB216763. The disease phenotype in mammalian cardiac myocytes and its rapid reversal by SB216763 are associated with marked changes in the subcellular distribution of critical cell-cell junction proteins but without a change in the total cellular content of these proteins, strongly implicating a trafficking defect mediated by altered Wnt signaling. In further support of this mechanism is data in both mammalian cardiac myocytes and in patient samples that abnormal distribution of the PDZ protein SAP97, implicated in forward trafficking of Nav1.5 and Kir2.1,⁸ is a component of the fundamental disease pathway in AC. Taken together, these observations provide new insights into the pathogenesis of AC and link myocyte injury and electrophysiological derangements to a common disease mechanism.

Materials and Methods

Neonatal rat ventricular myocyte isolation and culture, stretch protocols: Primary cardiac myocyte cultures were prepared from ventricles of 1-day-old Wistar rat pups (Charles River) as previously described.⁹ Cells were plated on collagen-coated plastic chamber slides at a density of 2.4×10′ cells/cm² and grown for 4 days prior to experimentation. In selected experiments, monolayers grown on collagen--coated silicone membranes were subjected to linear pulsatile stretch using a custom-designed apparatus as described in previous studies.'° Cells were stretched to 110% of resting length at a. frequency of 3 Hz for 1-4 hr in serum-free medium. In other experiments cultures were incubated with reagents including pifitlirin A (5 μM, 1 hr) and SB216763 (5 μM, 24 hr) prior to experimentation. All protocols were approved by the Institutional Animal Care and Use Committee (IACUC).

Viral transfection: A recombinant adenoviral construct expressing 2057del2-plakoglobin was created with the ViraPower Adenoviral Expression system (Invitrogen) using the pAd/CMV/V5-DEST vector through LR clonase-mediated recombination. The V5-tag was used to identify the mutant protein and distinguish it from wildtype plakoglobin. Two days post-plating, monolayers were exposed to viral solution (1:130 dilution in serum-free media, 37° C.) for 1 hr after which viral solution was replaced with complete medium in the presence or absence of reagents.

Immunofluoresence staining and microscopy in cultured cells: Cultures were rinsed in serum-free medium and fixed in 4% paraformaldehyde at room temperature for 5 min or in acetone at −20° C. for 10 min. Fixed cells were immunostained with mouse monoclonal anti-plakoglobin (Sigma), anti-N-cadhcrin (Sigma), anti-Cx43 (Millipore), anti-desmopla.kin (Fitzgerald), anti-SAP97 (Santa Cruz Biotechnology) antibodies and anti-Nav1.5 (kindly provided by Dr. Hugues Abriel, University of Bern, Switzerland) antibodies. Specific immunoreactive signal was detected by laser scanning confocal microscopy as previously described.”

Immuno, fluorescence staining and microscopy in patient myocardium: Formalin-fixed, paraffin-embedded blocks of right ventricular myocardium were analyzed in eight patients with ARVC in whom the disease was confirmed pathologically at autopsy in seven and by endomyocardial biopsy in one. A desmosomal gene mutation had been identified in six of these patients (DSP: R1113X or 1218+1G→A; PKP2: 2506delA or 2146-1G→C; JUP: S39_K40insS; DSG2: C591X), whereas in the remaining two cases no mutation was identified in any of the candidate genes screened but histology was consistent with ARVC. The second set of samples came from the native hearts of patients who had undergone cardiac transplantation. Transmural sections of right and left ventricles from each of five patients with end-stage hypertrophic, dilated, or ischemic cardiomyopathies were analyzed. Controls consisted of myocardium obtained at autopsy from 5 patients in whom there was no clinical or pathological evidence of heart disease. In preparation for immunofluorescence microscopy, deparaffinized, rehydrated slide-mounted sections were heated in citrate buffer (10mmol/L, pH 6.0), and after being cooled to room temperature, were simultaneously permeabilized and blocked in phosphate-buffered saline (PBS) containing 1% Triton X-100, 3% normal goat serum and 1% bovine serum albumin. The sections were then incubated with a primary anti-SAP97 antibody (rabbit polyclonal, Santa Cruz Biotechnology) and thereafter with indocarbocyanine-conjugated goat anti-rabbit IgG. Immunostained preparations were analyzed by confocal microscopy (Zeiss, LSM-510) as described previously.¹¹

Western Immunoblotting: Cells were washed in PBS and scraped from culture dishes in a low ionic strength buffer containing protease inhibitors (1 mM NaHCO₃, 5 mM EDTA, 1 mM EGTA, 1 μM leupeptin, 1 μM pepstatin, 0.1 μM aprotinin, 1 mM benzamidine, 1 mM iodoacetamide, 1 mM phenylmethylsulfortylfluoride). Following centrifugation, the pellet was resuspended in the same buffer and aliquots containing 10 μg of total protein were analyzed by SDS polyacrylamide gel electrophoresis. Proteins were detected by enhanced chemiluminescence (ECL. Amersham Corp). All blots were stripped in 62.5 mM Tris-HCl, pH 6.8, 100 mM β-mercapto-ethanol, and re-probed with a mouse monoclonal anti-GAPDH antibody (Fitzgerald) as a loading control.

Caspase-3 assay: Caspase-3 activity was assessed by a colorimetric assay (Calbiochem). Control myocytes and myocytes expressing 2057del2 plakoglobin were subjected to 4 hr of cyclical, uniaxial stretch (110% of resting length at 3Hz), washed in PBS and collected by trypsinization followed by centrifugation. The cellular pellet was resuspended in cell lysis buffer and incubated on ice for 10 min. Lysates were centrifuged for 5 min at 13,000 g, and the supernates were assayed for caspase-3 activity in assay buffer (50 mM HEPES, pH 7.4, 100 mM NaCl, 0.1% 3-[(3-cholamidopropyl) dimethylammonio]-1-propanesulfonate, 10 mM dithiothreitol, 0.1 mM EDTA, 10% glycerol). After addition of the caspase substrate DEVC (2nM), samples were incubated for 60 min at 37° C. and read at 405 nm (Bio-Tek Instruments). Cultures not subjected to stretch were also assayed for caspase-3 activity for control purposes.

TUNEL assay: Apoptotic cells in cultures were detected by terminal deoxynucleotidyl transferase-mediated biotinylated UTP nick end-labeling (TUNEL) assay using the ApopTag™ Fluorescein Direct in Situ Apoptosis Detection Kit (Millipore) as previously described.⁹ Nuclei were stained with DAPI, and TUNEL-positive and total nuclei were counted under a laser scanning confocal microscope in 5 fields per chamber.

Cytokine expression: Conditioned culture media (after 24 or 48 hr) from control cells and cells expressing 2057del2-plakoglobin in the presence or absence of SB216763 (5 μM, 24 hr) were collected, mixed with a cocktail of biotinylated detection antibodies and incubated with nitro-cellulose membranes spotted in duplicate with control and capture antibodies (R&D Systems). Chemiluminescent signal produced at each spot corresponded to the amount of cytokine bound. Conditioned media from control, un-transfected cultures were assayed for cytokine expression for control purposes.

Zebrafish ventricular myocyte isolation and culture: Zebrafish (Danio rerio) were bred and maintained at 28′C with 14 hr exposure to light and 10 hr to dark (day/night cycle) according to IACUC-approved protocols. Hearts were extracted from anesthetized fish at 21-56 days post-fertilization(dpf) using micro-dissecting tweezers (Roboz Surgical Instruments), washed vigorously in culture medium (M199, primocin, HEPES, L-glutamine, 10% fetal bovine serum) and placed in fresh medium. Ventricles were separated at the atrio-ventricular junction, pooled in a tube containing enzyme solution (HBSS without calcium/magnesium, Trypsin, PCN/Streptomycin, HEPES) at 37° C. and agitated in a thermomixer at 1000-1200 RPM. Dissociated cells were collected every 10 min from supernatants and transferred to tubes containing culture medium and 10% fetal bovine srum to halt trypsinization. Cells were then pelleted by centrifugation, resuspended in fresh culture medium, and seeded on sterile cover slips coated with laminin (20 μg/ml)¹¹ at 28° C. in a 5% CO₂ incubator. After 36 hr, culture medium was changed and unattached cells were removed.

Cellular electrophysiology methods: Current clamp experiments in isolated neonatal rat and zebrasfish ventricular myocytes were carried out using standard protocols.¹² Micropipettes had a tip resistance of 3-5 MΩ. After opening the gigaseal, the microelectrode amplifier (HEKA, EP-10, Lambrecht, Germany) was set to zero holding current and cells were stimulated at a frequency of 0.5 to 1 Hz. Recordings were corrected for a junction potential of 12 mV.^{12,13}

I_{Na} and I_{Na} activation and inactivation were measured by whole-cell voltage-clamp according to standard protocols.^{14,15} Cells were held at a membrane potential of −80 mV after which pulses between -120 and +50 mV were applied in 5 mV steps each with a duration of 250 ms. Inactivation was measured from membrane potentials between −120 mV and +50 mV at maximal activation.^{14,15} In view of the small cell size (cell capacitance of 6.7±2.6pF; n=47) it was not necessary to reduce extracellular [Nal. Access resistance of the patch electrodes was <7 MΩ Measurements showing incomplete control of membrane potential were eliminated from analysis.^{14,15}

The inward rectifier current I_{K1} was measured using the protocol described by Dobrev.^{16,17} Ramp clamps of 1400 ms duration were applied between −120 and +20 mV. After each clamp, external solution containing Ba²⁺(1 mM) was applied via a large pore pipette placed adjacent to the clamped cell. I_{K1} was defined as the total current minus the Ba²⁺-insensitive current. ¹⁶ Subtraction of the two currents was performed using a MatLab™ script.

The outward K⁺ current k_{Kr} was measured according to the protocol of Nemtsas et al. using identical extracellular and pipette solutions.” In brief, I_{K}, was activated by clamping the membrane potential from a holding potential of −50 mV for 1000 ms to different levels in 5 mV steps. I_{Kr} was obtained as the tail current upon repolarization to −50 mV after the 1000 ms conditioning steps. Contaminating I_{CaL} was inhibited by nisoldipine (1 μM). Addition of the specific I_{Kr} blocker E4031 identified the tail current as I_{Kr}.¹⁷

Statistical analysis: Data were analyzed using ANOVA or Student's unpaired t-test, where appropriate. Data are expressed as mean ±SE.

Results

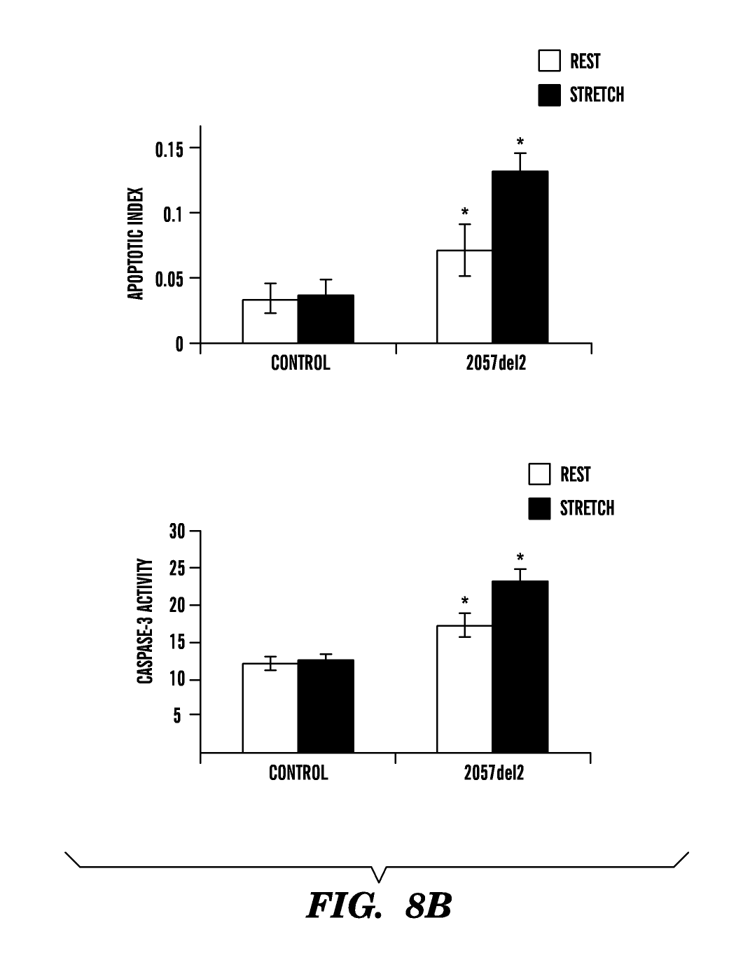

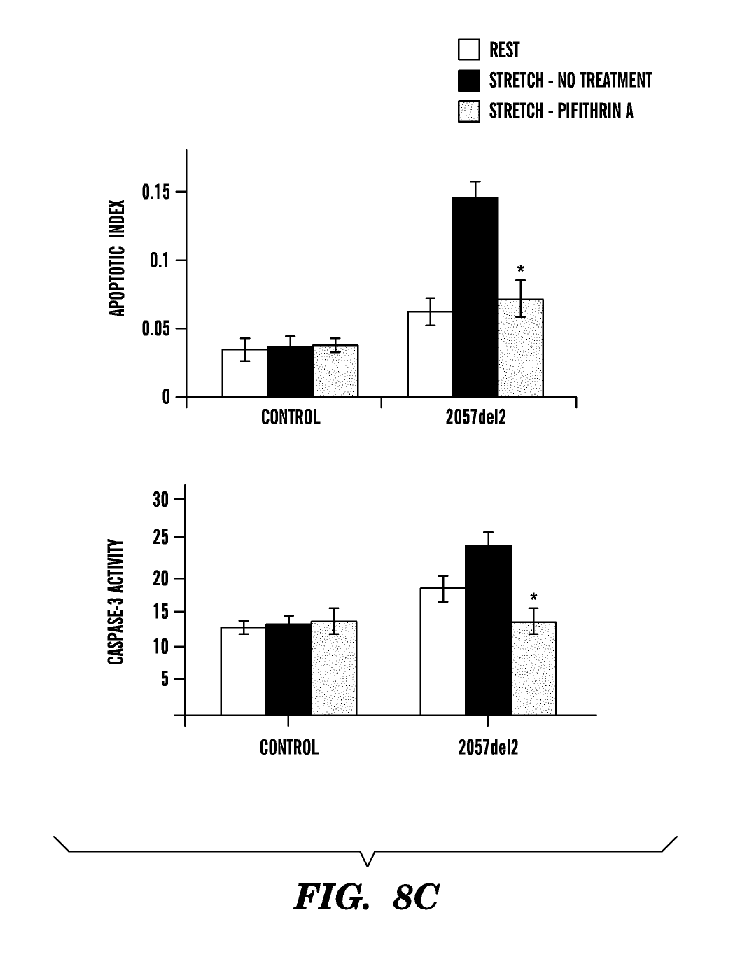

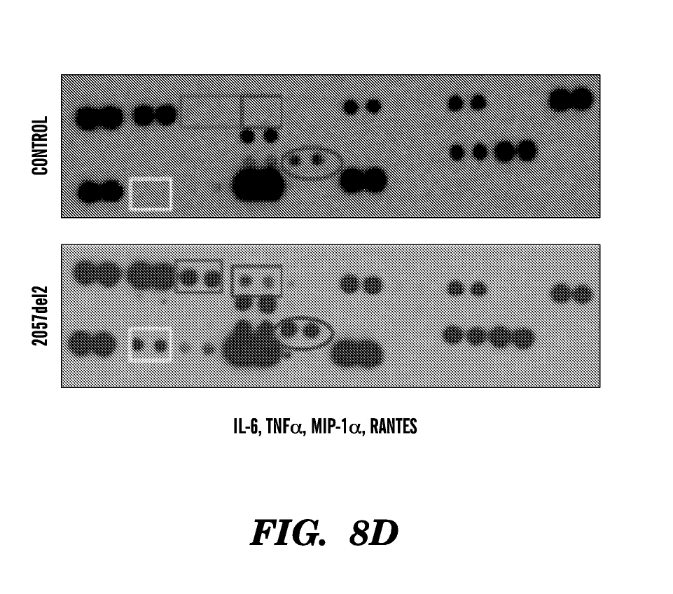

An in vitro model of AC recapitulates features observed in patients: Previous studies of human myocardium have identified 4 features that appear to play a role in the pathogenesis of AC in patients. These include: 1) decreased immunoreactive signal for plakoglobin (γ-catenin) at cell-cell junctions;³¹ 2) gap junction remodeling indicated by decreased immunoreactive signal for the major ventricular gap junction protein, connexin43 (Cx43), at cell-cell junctions;” 3) myocardial apoptosis;¹⁸ and 4) high circulating levels of pro-inflammatory cytokines and expression of cytokines by cardiac myocytes.¹⁹ To gain insights into mechanisms responsible for these features of AC in patients, an in vitro model in which normal neonatal rat ventricular myocytes were transfected with adenovirus to express 2057del2 plakoglobin with a V5 epitope tag was developed. Conditions yielding >95% transfection (assessed using adenovirus containing GFP) and expression of 2057del2 plakoglobin at levels roughly equivalent to that of the endogenous normal protein were identified (FIGS. 8A-8D). The effects of transgene expression on development of features identified in the human disease were then assessed. As illustrated in FIGS. 8A-8D, expression of 2057del2 plakoglobin for 24 hr led to 1) a marked change in the distribution of immunoreactive signal for plakoglobin with diminished signal at cell-cell junctions and abundant signal in nuclei; 2) greatly reduced immunoreactive signal for Cx43 at cell-cell junctions; and 3) increased myocyte apoptosis indicated by greater numbers of TUNEL-positive nuclei and expression of caspase-3. Apoptosis in cells expressing 2057del2 plakoglobin could be prevented by pifithrin-A (5 μM) which inhibits p53 transcriptional activity, or greatly increased by subjecting cells to brief intervals of uniaxial cyclical stretch (FIGS. 8A-8D). Finally, cells transfected to express 2057del2 plakoglobin for 24 hr secreted various inflammatory mediators into the culture medium including IL-6, TNFα, MIP1α, RANTES, and IL-17, several of which have been identified in the blood or myocardium in patients with AC¹⁸ (FIGS. 8A-8D). Taken together, these results show that expression of 2057del2 plakoglobin by neonatal rat ventricular myocytes recapitulates features consistently identified in the hearts of patients with AC.

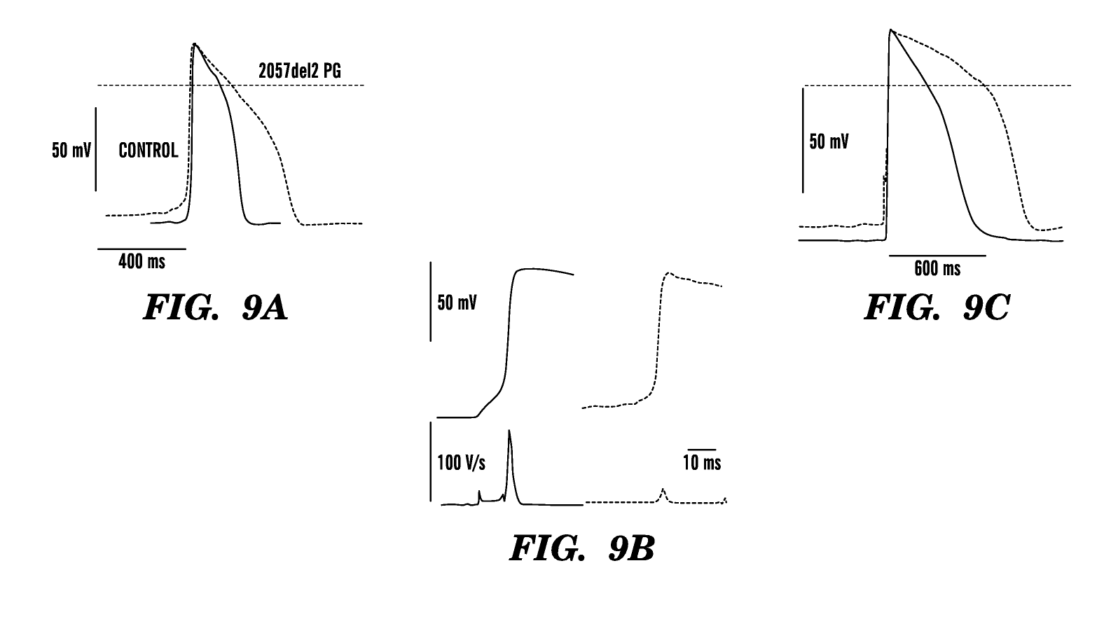

Cellular electrophysiology of zebrafish ventricular myocytes expressing 2057del2 plakoglobin: Methods for the isolation and long-term culture of ventricular myocytes from zebrafish were developed, and current-clamp and whole-cell voltage-clamp methods used to characterize action potentials and changes in I_{Na}, I_{K1} and I_{Kr}. As shown in FIGS. 9A-9C and Table 1, marked changes in action potential morphology were observed in myocytes obtained from transgenic fish at 5-7 weeks post-fertilization compared with myocytes from control fish (either wildtype or those expressing Ga14-VP 16 but not the responder construct to drive transgene expression). Resting membrane potential (RMP) was significantly depolarized in cells expressing 2057del2 plakoglobin compared with controls (−69±1 vs. −79±1 mV, respectively, n=12 or 11; p<0.001). The maximum rate of rise in voltage during phase 1 of the action potential, dV/dtₘₐₓ, was markedly blunted (18±2 vs. 63±11 V/sec, n=12 or 10; p<0.001), and action potential duration at 80% completion of repolarization (APD_{8O}%) was prolonged (319±44 vs. 240±24 ms, n=12 or 11; p<0.05). Nearly identical changes in action potential shape were observed in myocytes isolated from fish 3-4 weeks post-fertilization, indicating no major progression in the action potential phenotype between 3 and 7 weeks of age (Table 1). Furthermore, virtually identical changes in action potential morphology (positive shift in RMP, reduction of dV/dtₘₐₓ and prolongation of APD₈₀%) were observed in neonatal rat ventricular myocytes expressing 2057del2 plakoglobin. This observation suggests that the action potential phenotype observed in the fish model reflects critical features of disease exhibited by mammalian cardiac myocytes expressing a known human AC disease gene, and presumably, by ventricular myocytes in patients with AC.

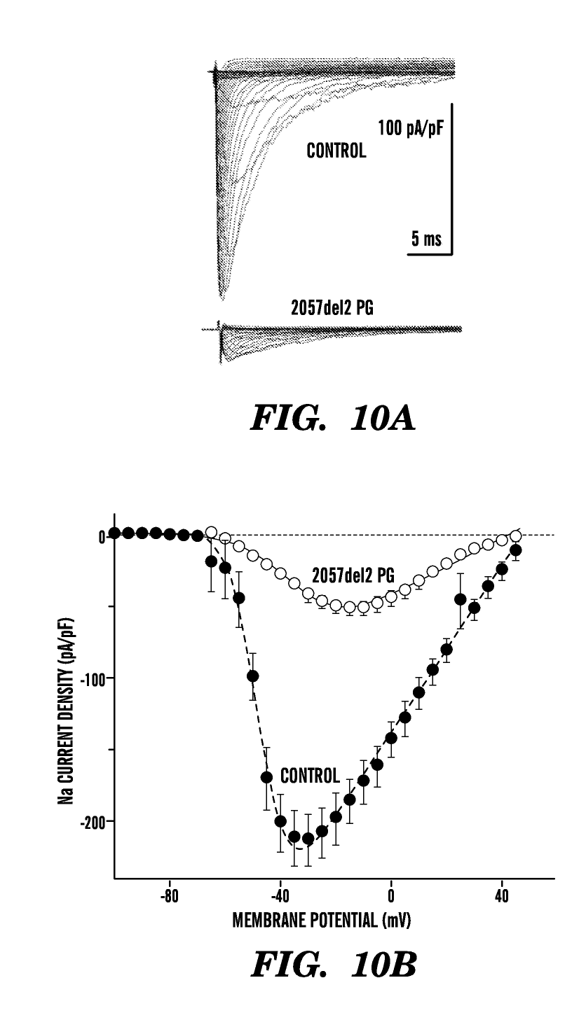

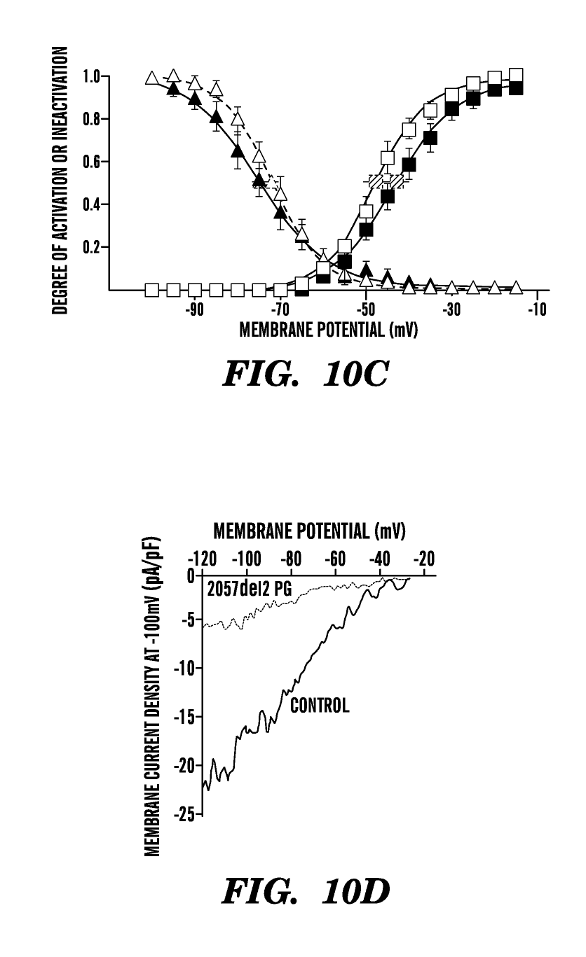

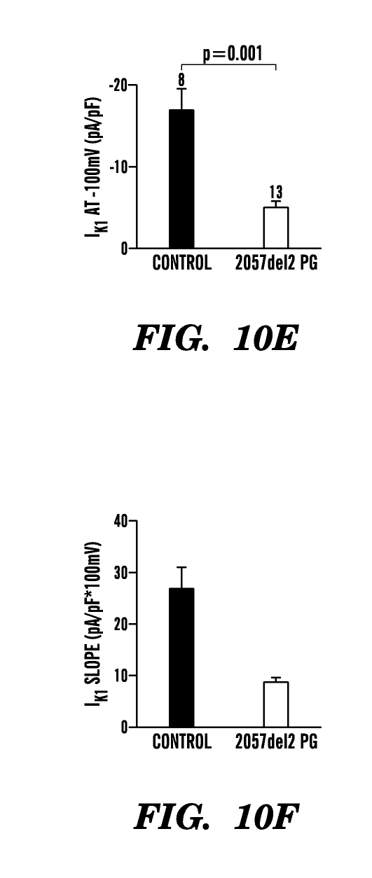

The marked decrement in dV/dtₘₐₓ in phase 1 of the action potential prompted us to characterize potential changes in I_{Na} in zebrafish ventricular myocytes expressing 2057del2 plakoglobin. Using whole-cell voltage-clamp and starting from a holding potential of −80 mV, we observed a large reduction in I_{Na} current density across a broad range of membrane potentials (FIG. 3 and Table 2). Peak I_{Na} current density was reduced by 84%, from 196±22 pA/pF in control cells (n=18) to 31±3 pA/pF (n=16; p <0.001), in myocytes expressing 2057del2 plakoglobin isolated from fish at 5-7 weeks post-fertilization. Similar reduction (76%) was observed in myocytes isolated from fish at 2-3 weeks post-fertilization (Table 2). No significant shift was seen in the steady-state activation and inactivation curves of I_{Na} (FIG. 10C). Thus, the marked reduction in I_{Na} current density in fish myocytes expressing 2057del2 plakoglobin appears to be due solely to a reduction in the number of functional channels.

The more depolarized resting membrane potential in myocytes expressing mutant plakoglobin raised the possibility of alterations in I_{Ki}, a current known to be important in maintaining normal resting potential. Indeed, a similar marked decrease (71%) in I_{K1} current density at −100 mV (from 17.0±2.7 pA/pF to 5±0.8 pA/pF, n=8 and 13 respectively; p<0.001) was observed in cells expressing 2057del2 plakoglobin (FIGS. 10D-10F and Table 2). However, no difference was observed in I_{K} current density between controls and myocytes expressing 2057del2 plakoglobin (and Table 2).

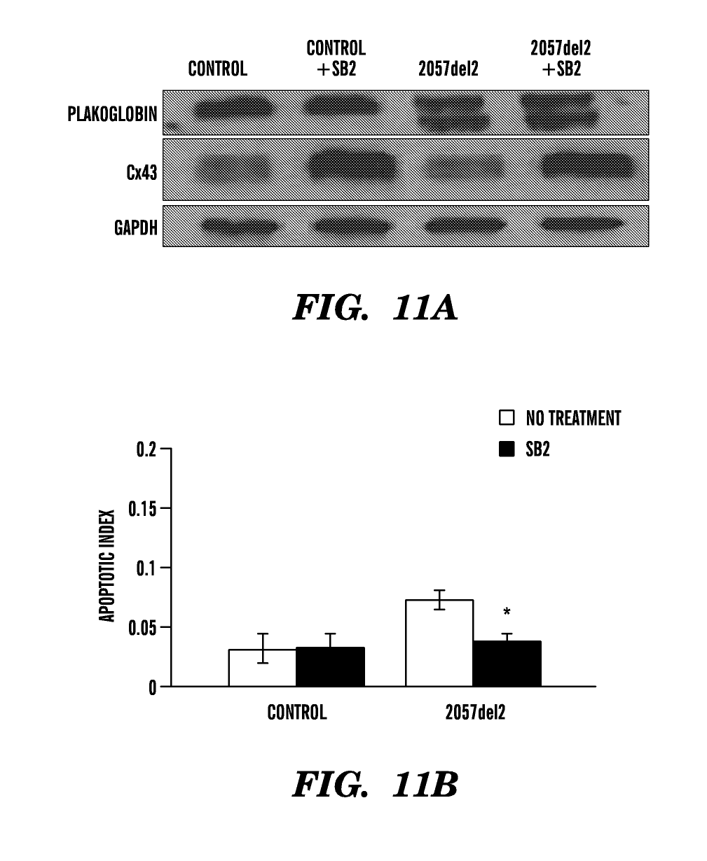

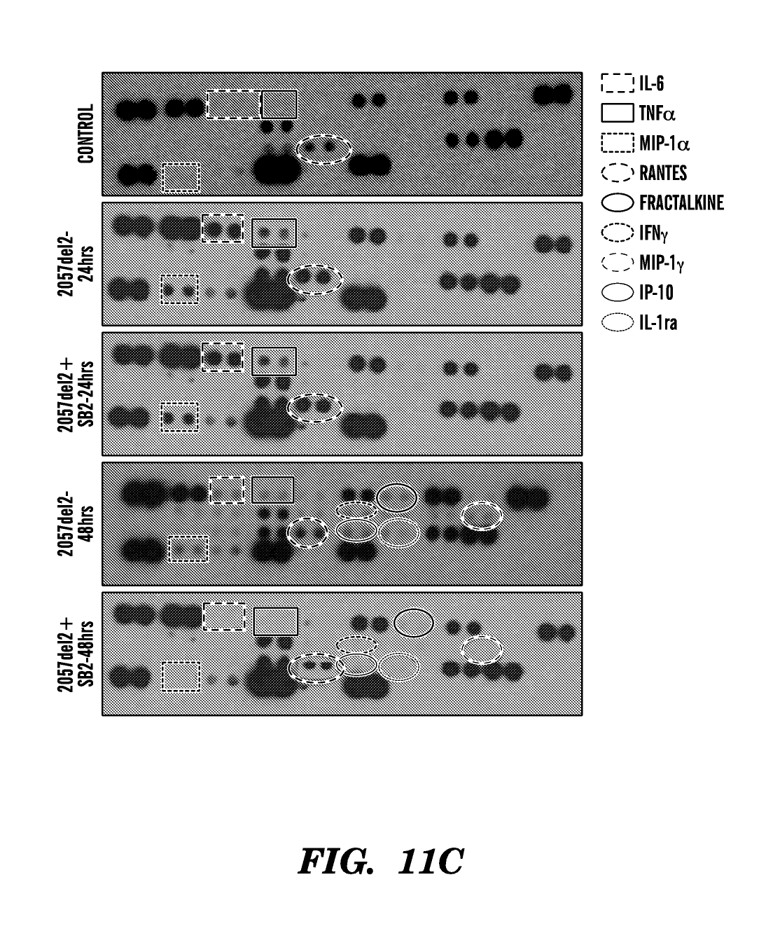

Reversal of disease features by SB216763 in mammalian myocytes expressing 2057del2 plakoglobin: To determine whether SB216763 is capable of mitigating the effects of 2057del2 plakoglobin expression in mammalian cardiac myocytes, cells were transfected and 24 hr later monolayers exposed to SB216763 (5 μM) for an additional 24 hr. As illustrated in FIGS. 11A-11C, SB216763 prevented the marked change in subcellular distribution of plakoglobin and accumulation of nuclear signal seen in cells expressing mutant plakoglobin. It also prevented remodeling of gap junctions as indicated by the presence of control levels of junctional Cx43 signal in treated cells, and it actually increased the amount of Cx43 signal at cell-cell junctions in control (non-transfected) cultures (FIGS. 11A-11C). The marked changes in the distribution of immunoreactive signals for plakoglobin and Cx43 and the nearly complete reversal of these changes in cells expressing 2507del2 plakoglobin occurred with no apparent change in the total cellular content of plakoglobin and an increase in the total cellular content of Cx43 (both in control and transfected myocytes) as assessed by immunoblotting (FIGS. 11A-11C). These observations, coupled with the rapidity with which these proteins changed their subcellular distribution, indicate that 2057del2 disturbs the normal trafficking of plakoglobin and Cx43 to the cell surface, and SB216763 corrects this defect. SB216763 also dramatically reduced the number of TUNEL positive nuclei in myocytes expressing mutant plakoglobin (FIGS. 11A-11C). Finally, there was no apparent effect on cytokines accumulating in the culture medium in transfected cells incubated with SB216763 for 24 hr (data not shown) but there was a clear reduction in cytokine levels in media recovered from cells exposed to SB216763 for 48 hr (FIG. 11A-11C). An even greater diversity of cytokines was identified in the culture medium from cells expressing 2057del2 plakoglobin for 48 vs. 24 hr, but the amounts were all reduced by SB216763. Taken together, these observations indicate that changes in the distribution of critical junctional proteins, expression of inflammatory markers of cell injury and apoptosis of cardiac myocytes are all mediated by a common disease pathway that can corrected by SB216763.

Confocal immunofluorescence images revealed plakoglobin and Cx43 immunoreactive signal distribution in NRVMs in the presence or absence of SB216763 (data not shown). SB216763 prevented nuclear accumulation of plakoglobin signal and increased junctional signal for Cx43 in NRVMs expressing 2057del2 plakoglobin. SB216763 also increased junctional signal for Cx43 in control, un-transfected cultures.

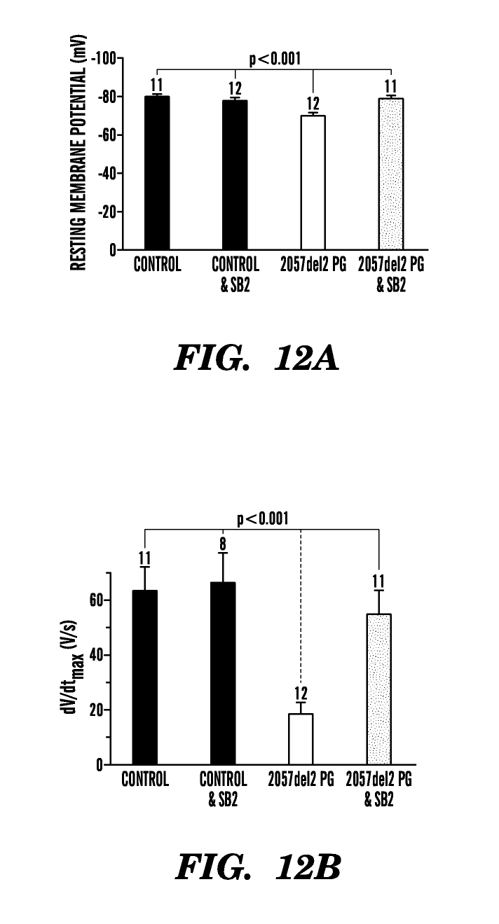

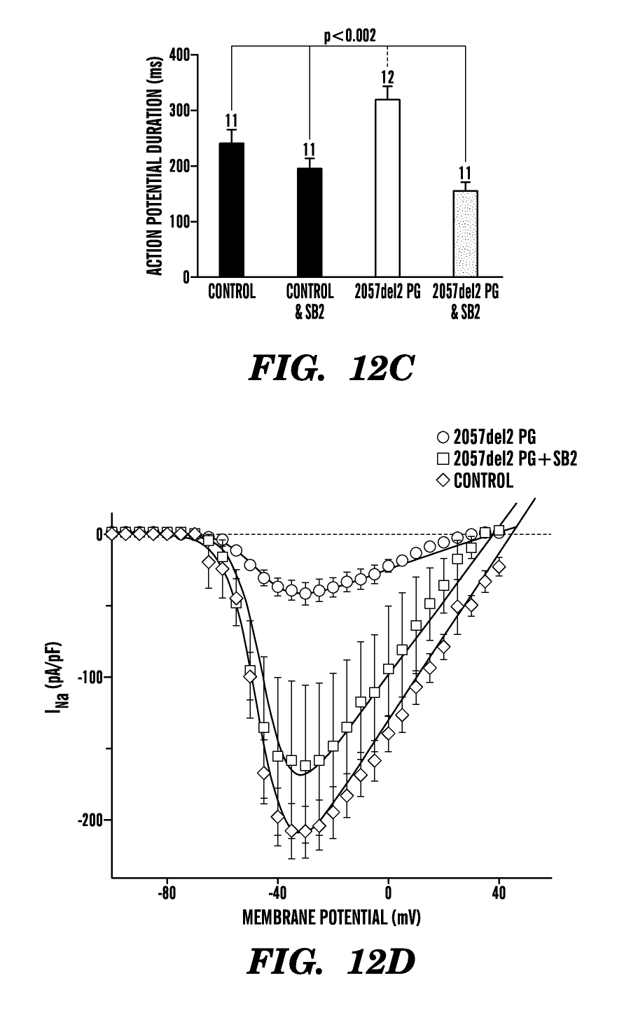

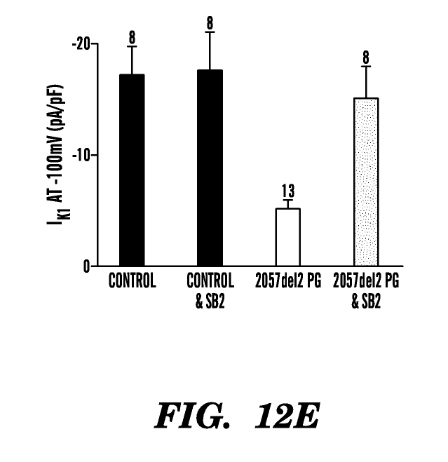

Reversal of electrophysiological derangements by SB216763 in cells expressing 2057del2 plakoglobin: To determine whether SB216763 affects action potential remodeling and changes in I_{Na} and I_{K1} observed in zebrafish ventricular myocyte cells expressing 2057del2 plakoglobin, cellular electrophysiology was characterized in isolated myocytes exposed to SB216763 at 2 different developmental stages. In the first protocol, zebrafish embryos at 1 dpf were treated with SB216763 for 6 days (3.0 mmol/L) and then maintained under normal conditions (in the absence of SB2) until 5-7 weeks post-fertilization at which time ventricular myocytes were isolated and studied by current-clamp and whole-cell voltage-clamp methods. As shown in FIGS. 12A-12E and Table 1, SB216763 had no effect on action potentials in myocytes from control fish, whereas it normalized RMP and dV/dtₘₐₓ, and actually shortened APD_{8O}% beyond control levels in fish expressing mutant plakoglobin. Similarly, early exposure to SB216763 had no effect on I_{Na} or I_{K1} current densities in control myocytes but caused marked normalization of these currents in cells expressing 2057del2 plakoglobin (FIGS. 12A-12E and Table 2). Thus, transient exposure to SB216763 during early embryonic development when cardiac morphogenesis is occurring durably prevents action potential remodeling in adult fish expressing mutant plakoglobin. To determine whether SB216763 can reverse these changes once they occur, a second protocol was performed in which isolated ventricular myocytes were prepared from transgenic fish at 3-4 weeks post-fertilization (when the action potential phenotype is fully developed) and then treated in culture with SB216763 for 36 hr before being analyzed. As shown in FIGS. 12A-12E and Tables 1 and 2, short term exposure of isolated cells to SB216763 normalized action potential remodeling and reversed reductions in I_{Na} and I_{K1} current densities. The fact these changes can be normalized in isolated cells in vitro indicates that the marked electrical phenotype produced by expression of an AC disease gene is myocyte autonomous. Moreover, the electrical phenotype can apparently be prevented from occurring in adult fish when early embryos are transiently exposed to SB216763, and once it becomes fully developed in adult fish, it can be reversed by exposing SB216763.

Effects of 2057del2 plakoglobin on expression and distribution of SAP97 and Nav1.5: The PDZ protein SAP97 has been shown to regulate normal targeting to the cell surface of Nav1.5 and Kir2.1,⁸ the major protein subunits responsible for I_{Na} and I_{K1}, respectively. To determine whether derangements in this trafficking pathway might account for the rapidly reversible reductions in I_{Na} and I_{K1} observed in myocytes expressing mutant plakoglobin, immunohistochemistry and immunoblotting were used to characterize the distribution and total cellular content of SAP97 in neonatal rat ventricular myocytes transfected to express 2057del2 plakoglobin Immunoreactive signal for SAP97 was concentrated at the cell surface in control myocytes, similar to the previously reported localization of this protein to intercalated disks in adult rat ventricular myocardium.⁸ Confocal immunofluorescence images showed the distribution of SAP97 and Nav1.5 in control myocytes and myocytes expressing 2057del2 plakoglobin in the presence or absence of SB216763. SAP97 localizes mainly at cell-cell junctions in control myocytes. Junctional signal for SAP97 was greatly reduced in myocytes expressing 2057del2 plakoglobin but a more normal distribution was restored following exposure to SB216763 for 24 hr. Nav1.5 signal was present at cell-cell junctions and within controls cells. Total signal was greatly reduced in myocytes expressing 2057del2 plakoglobin, whereas signal levels were restored following exposure to SB216763 for 24 hr.

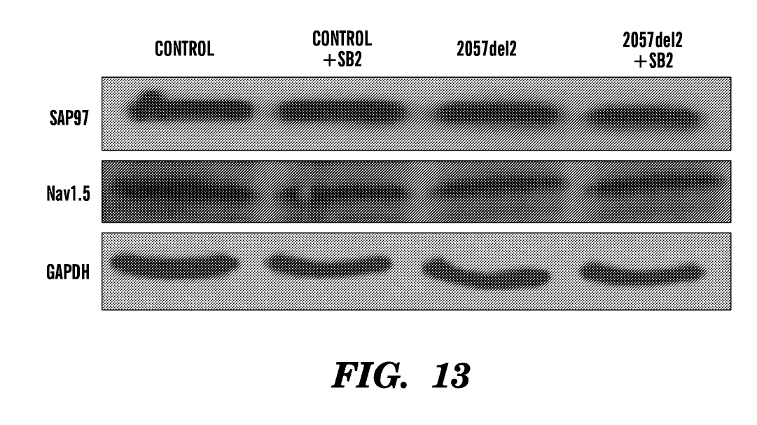

However, there was a marked decrease in the amount of SAP97 signal at the cell surface in myocytes expressing 2057del2 plakoglobin, which was fully reversed by exposing myocytes to SB216763 for 24 hr (data not shown). These rapidly reversible changes in subcellular distribution of SAP97 immunoreactive signal occurred without apparent changes in the total amount of SAP97 within the myocytes as shown by western blots (FIG. 13). Taken together with the previous electrophysiological studies, these results strongly implicate defective trafficking of ion channel proteins as the mechanism underlying action potential remodeling and reduced altered I_{Na} and I_{K1} current densities in ventricular myocytes expressing 2057del2 plakoglobin. To provide additional independent evidence in support of this mechanism, we characterized the distribution and total cellular content of Nav1.5 in neonatal rat myocytes. As shown in FIG. 6, there was a marked reduction in immunoreactive signal for Nav1.5 in myocytes expressing mutant plakoglobin, and this was reversed in cells exposed to SB216763 for 24 hr. Yet despite these marked changes in the apparent amount and distribution of Navl 5 immunoreactive signal, there was no difference in the total content of Nav1.5 protein assessed by immunoblotting in control cells or cells expressing mutant plakoglobin, nor did exposure to SB216763 affect overall levels of Nav1.5 (data shown). These observations provide additional independent evidence that changes in cellular electrophysiology in AC are related to defective forward trafficking of key ion channel proteins rather than to insufficient channel protein production. They also suggest a common disease pathway, sensitive to the mitigating effects of SB2, underlying both myocyte injury/apoptosis and arrhythmias in AC.

Abnormal distribution of SAP97 in myocardium from patients with AC: To determine whether insights into disease mechanisms gained from analysis of zebrafish myocytes and confirmed in mammalian myocytes in vitro apply to the actual human disease, sections of formalin-fixed paraffin-embedded myocardium from patients with AC were immunostained using anti-SAP97 antibodies. Ttissue from four control subjects (non-cardiac deaths) and eight patients with documented AC including two each with mutations in PKP2 or DSP, one each with mutations in JUP or DSG2, and two patients with clinical expression of disease but no apparent desmosomal gene mutation were analyzed. Control human myocardium showed immunoreactive signal concentrated at intercalated disks and in a sarcomeric pattern, identical to that shown previously in adult rat ventricular myocardium.⁸ By contrast, a marked reduction in SAP97 signal was seen in the ventricular myocardium of patients with AC independent of the specific mutation involved in causing their disease (attempts to analyze the distribution of Nav1.5 in patient material were unsuccessful because the available antibodies did not work in formalin-fixed paraffin-embedded tissues). To determine whether reduced SAP97 signal is specific for AC, 15 myocardial samples from patients with end-stage ischemic, dilated or hypertrophic cardiomyopathy were stained. As seen in representative cases, the myocardium in these other forms of human heart disease showed near-control levels of SAP97 signal in the sarcomeric distribution (although there did seem to be relative loss of signal at intercalated disks). Thus, SAP97 immunoreactive signal seems to be preferentially decreased in the hearts of patients with AC compared to other forms of heart disease. These results implicate deranged SAP97 function in a specific form of human heart disease and validate observations in model systems of AC and raise the possibility that SAP97 may be a new biomarker of disease severity and arrhythmogenesis in AC.

Discussion:

AC is a deadly disease for which no mechanism-based therapies exist.^{1,2} To understand why it is so arrhythmogenic and to identify potential drug candidates, a zebrafish model of AC was used for high-throughput screening to identify a small molecule that rescues the disease phenotype and reduces mortality.⁴ Described herein is the use of this model to identify marked abnormalities in action potentials and ionic currents in fish ventricular myocytes which likely contribute to arrhythmogenesis in AC. Armed with information from studies in fish, these observations were confirmed and extended in studies of mammalian ventricular myocytes to show that myocyte injury and arrhythmogenesis are linked to a common disease pathway. Insights gained from studies in fish and rat myocytes ultimately led to identification of SAP97 as a potential new biomarker in AC patients that appears to be part of this disease pathway.

Several aspects of this study require discussion. The 2057del2 mutation in plakoglobin was used to model human AC in fish and neonatal rat systems. At first glance, this may appear to be a poor choice. 2057del2 plakoglobin causes Naxos disease, a cardiocutaneous syndrome with recessive inheritance,^{5,6} whereas transgenic approaches were used in fish and rat myocytes to express 2057del2 plakoglobin in addition to the wildtype allele. Despite its recessive inheritance, however, Naxos disease is not caused by deficient expression of plakoglobin (which when knocked-out in mice leads to embryonic lethality). Rather, mutant plakoglobin is expressed in Naxos patients²⁰ and the disease phenotype apparently depends on gene-dosage effects. Another reason for choosing 2057del2 plakoglobin to model human disease is the highly penetrant nature of the classical ARVC phenotype in Naxos disease. The full penetrance suggests that expression of sufficient 2057del2 plakoglobin activates the critical disease pathway and can overcome potential mitigating effects of powerful modifiers implicated in the variable penetrance associated with other mutations in AC. Most importantly, it is demonstrated herein that neonatal rat myocytes expressing 2057del2 plakoglobin faithfully recapitulate consistent features of the human disease which occur independent of specific disease alleles: redistribution of plakoglobin from junctional to intracellular sites, remodeling of gap junctions, increased myocyte apoptosis, and expression of inflammatory mediator of disease.

Another aspect of this study worth noting is the highly abnormal response to mechanical stress in cells expressing 2057del2 plakoglobin. Normal neonatal rat ventricular myocytes respond to brief intervals of cyclical stretch by rapidly increasing levels of immunoreactive signals for proteins at cell-cell junction including plakoglobin and Cx43.²¹ By contrast, myocytes expressing 2057del2 plakoglobin fail to increase cell-cell junction proteins in response to stretch. Instead, they exhibit greatly increased rates of apoptosis after being stretched, whereas control cells show no appreciable apoptosis. These observations are consistent with clinical experience documenting disease flairs in AC patients following strenuous exercise²² during which the RV typically undergoes significant dilatation. Taken together, these observations indicate that zebrafish and mammalian myocyte models involving transgenic expression of 2057del2 plakoglobin are highly appropriate experimental systems in which to elucidate disease mechanisms in human AC. Perhaps the most convincing justification for this claim is that observations in fish myocytes expressing 2057del2 plakoglobin, confirmed and extended in studies of rat myocytes 2057del2 plakoglobin, led directly to the discovery of abnormal SAP97 signal in the hearts of patients with AC.

Reduced I_{Na} current density has been observed previously in neonatal rat myocytes following knock-down of plakophilin2²³ and in a transgenic mouse model with cardiac myocyte expression of mutant desmoglein-2.²⁴ The present observations demonstrate that, at least in the fish model, reduced I_{Na} current density is attributable largely if not entirely to a reduced number of functional channels at the cell surface rather than to changes in activation/inactivation kinetics as reported in cells following knockdown of plakophilin. This conclusion is supported by reduced immunosignal for Nav1.5 in neonatal rat myocytes expressing mutant plakoglobin, an observation we have also recently reported in patients with AC.²⁵ The finding of reduced I_{Ki}current density is new. This not only implicates another potentially highly arrhythmogenic electrophysiological defect in AC, but also led to discovery of aberrant SAP97 distribution (and presumably function) in myocytes expressing mutant plakoglobin and in AC patients whose disease is linked to various mutations in desmosomal genes. This observation is particularly intriguing because the PDZ protein SAP97 has been shown to associate with Nav1.5,¹⁵ and to regulate forward trafficking of Nav1.5 and Kir2.1 to the cell surface.⁸ These proteins form channels responsible for I_{Na} and I_{K1}, respectively, both of which were found to be dramatically reduced in fish myocytes expressing 2057del2 plakoglobin. No such reduction was seen, however, in I_{Kr} current density, a channel not associated with SAP97.²⁶ Taken together, these observations strongly implicate altered SAP97 trafficking as a critical component of the disease pathway in AC.

Another aspect requiring further comment is the remarkable ability of SB216763 to prevent or reverse features of the disease phenotype in both zebrafish and rat models of AC induced by expression of 2057del2 plakoglobin. SB216763 is known to activate the canonical Wnt signaling pathway by inhibiting glycogen synthase kinase-3β. Increasing evidence from mouse models of AC has implicated changes in Wnt signaling in disease pathogenesis,²⁷ but the underlying mechanisms are poorly understood and there has been little if any direct evidence to prove a causal relationship. Increased Cx43 expression was observed in both control myocytes and 2057del2 plakoglobin-expressing cells exposed to SB2, consistent with previous studies showing that Cx43 gene expression can be up-regulated by activation of the canonical Wnt signaling pathway.²⁸ These observations indicate the presence of a fundamental disease pathway in AC mediating diverse features of the disease including loss of plakoglobin from junctions, remodeling of gap junctions, myocyte injury/apoptosis and action potential remodeling. And, they further implicate a general mechanism involving abnormal forward trafficking of key desmosomal, gap junction and ion channel proteins to the intercalated disk. Perhaps most remarkably, it was observed that transient exposure to SB216763 of early zebrafish embryos undergoing cardiogenesis produces lasting effects to prevent action potential remodeling. Without wishing to be bound by theory, it is noted excessive mortality in fish with cardiac myocyte-specific expression of 2057del2 plakoglobin occurs within the first few days of development and levels off thereafter to control rates.⁴ This implies an early developmental effect of the disease pathway which, if blocked at a critical time, can prevent subsequent action potential remodeling. Yet, even after they become fully manifest, action potential remodeling and other features of myocyte injury can still be rescued by SB2, indicating ongoing reversibility of the effects of the common disease pathway.

### REFERENCES

1. Basso C, Corrado D, Marcus F I, Nava A, Thiene G. Arrhythmogenic right ventricular cardiomyopathy. Lancet. 2009; 373:1289-1300.
2. Saffitz J E. The pathobiology of arrhythmogenic cardiomyopathy. Annu Rev Pathol. 2011; 6:299-321.
3. Sen-Chowdhry S, Syrris P, Prasad S K, Hughes S E, Merrifield R, Ward D, Pennell D J, McKenna W J. Left-dominant arrhythmogenic cardiomyopathy: an under-recognized clinical entity. J Am Coll Cardiol. 2008; 52:2175-2187.
4. MacRae C et al. Zebrafish model paper
5. McKoy G, Protonotarios N, Crosby A, Tsatsopoulou A, Anastasakis A, Coonar A, Norman M, Baboonian C, Jeffery S, McKenna W J. Identification of a deletion in plakoglobin in arrhythmogenic right ventricular cardiomyopathy with palmoplantar keratoderma and woolly hair (naxos disease). Lancet. 2000; 355:2119-2124.
6. Protonotarios N, Tsatsopoulou A. Naxos disease and carvajal syndrome: Cardiocutaneous disorders that highlight the pathogenesis and broaden the spectrum of arrhythmogenic right ventricular cardiomyopathy. Cardiovasc Pathol. 2004; 13:185-194.
7. Becker J R, Robinson T Y, Sachidanandan C, Kelly A E, Coy S, Peterson R T, MacRae C A. In vivo natriuretic peptide reporter assay identifies chemical modifiers of hypertrophic cardiomyopathy signalling. Cardiovasc Res. 2012; 93:463-470.
8. Milstein M L, Musa H, Balbuena D P, Anumonwo J M, Auerbach D S, Furspan P B, Hou L, Hu B, Schumacher S M, Vaidyanathan R, Martens J R, Jalife J. Dynamic reciprocity of sodium and potassium channel expression in a macromolecular complex controls cardiac excitability and arrhythmia. Proc Natl Acad Sci USA. 2012; 109:E2134-2143.
9. Asimaki A, Syrris P, Wichter T, Matthias P, Saffitz J E, McKenna W J. A novel dominant mutation in plakoglobin causes arrhythmogenic right ventricular cardiomyopathy. Am J Hum Genet. 2007; 81:964-973.
10. Zhuang J, Yamada K A, Saffitz J E, Kleber A G. Pulsatile stretch remodels cell-to-cell communication in cultured myocytes. Circ Res. 2000; 87:316-322.
11. Asimaki A, Tandri H, Huang H, Halushka M K, Gautam S, Basso C, Thiene G, Tsatsopoulou A, Protonotarios N, McKenna W J, Calkins H, Saffitz J E. A new diagnostic test for arrhythmogenic right ventricular cardiomyopathy. N Engl J Med. 2009; 360:1075-1084.
12. Thomas S P, Bircher-Lehmann L, Thomas S A, Zhuang J, Saffitz J E, Kleber A G. Synthetic strands of neonatal mouse cardiac myocytes: Structural and electrophysiological properties. Circ Res. 2000; 87:467-473.
13. Neher E. Correction for liquid junction potentials in patch clamp experiments. Methods Enzymol. 1992; 207:123-131.
14. Gavillet B, Rougier J S, Domenighetti A A, Behar R, Boixel C, Ruchat P, Lehr H A, Pedrazzini T, Abriel H. Cardiac sodium channel nav1.5 is regulated by a multiprotein complex composed of syntrophins and dystrophin. Circ Res. 2006; 99:407-414.
15. Petitprez S, Zmoos A F, Ogrodnik J, Balse E, Raad N, El-Haou S, Albesa M, Bittihn P, Luther S, Lehnart S E, Hatem S N, Coulombe A, Abriel H. Sap97 and dystrophin macromolecular complexes determine two pools of cardiac sodium channels nav1.5 in cardiomyocytes. Circ Res. 2011; 108:294-304.
16. Dobrev D, Graf E, Wettwer E, Himmel H M, Hala O, Doerfel C, Christ T, Schuler S, Ravens U. Molecular basis of downregulation of G-protein-coupled inward rectifying K(+) current (I(K,ACh) in chronic human atrial fibrillation: Decrease in girk4 mRNA correlates with reduced I(K,ACh) and muscarinic receptor-mediated shortening of action potentials. Circulation. 2001; 104:2551-2557.
17. Nemtsas P, Wettwer E, Christ T, Weidinger G, Ravens U. Adult zebrafish heart as a model for human heart? An electrophysiological study. J Mol Cell Cardiol. 2010; 48:161-171.
18. Mallat Z, Tedgui A, Fontaliran F, Frank R, Durigon M, Fontaine G. Evidence of apoptosis in arrhythmogenic right ventricular dysplasia. N Engl J Med. 1996; 335:1190-1196.
19. Asimaki A, Tandri H, Duffy E R, Winterfield J R, Mackey-Bojack S, Picken M M, Cooper L T, Wilber D J, Marcus F I, Basso C, Thiene G, Tsatsopoulou A, Protonotarios N, Stevenson W G, McKenna W J, Gautam S, Remick D G, Calkins H, Saffitz J E. Altered desmosomal proteins in granulomatous myocarditis and potential pathogenic links to arrhythmogenic right ventricular cardiomyopathy. Circ Arrhythm Electrophysiol. 2011; 4:743-752.

20. Kaplan S R, Gard J J, Protonotarios N, Tsatsopoulou A, Spiliopoulou C, Anastasakis A, Squarcioni C P, McKenna W J, Thiene G, Basso C, Brousse N, Fontaine G, Saffitz J E. Remodeling of myocyte gap junctions in arrhythmogenic right ventricular cardiomyopathy due to a deletion in plakoglobin (naxos disease). Heart Rhythm. 2004; 1:3-11.
21. Yamada K, Green K G, Samarel A M, Saffitz J E. Distinct pathways regulate expression of cardiac electrical and mechanical junction proteins in response to stretch. Circ Res. 2005; 97:346-353.
22. Corrado D, Basso C, Pavei A, Michieli P, Schiavon M, Thiene G. Trends in sudden cardiovascular death in young competitive athletes after implementation of a preparticipation screening program. JAMA. 2006; 296:1593-1601.
23. Sato P Y, Musa H, Coombs W, Guerrero-Sema G, Patino G A, Taffet S M, Isom L L, Delmar M. Loss of plakophilin-2 expression leads to decreased sodium current and slower conduction velocity in cultured cardiac myocytes. Circ Res. 2009; 105:523-526.
24. Rizzo S, Lodder E M, Verkerk A O, Wolswinkel R, Beekman L, Pilichou K, Basso C, Remme C A, Thiene G, Bezzina C R. Intercalated disc abnormalities, reduced Na+ current density and conduction slowing in desmoglein-2 mutant mice prior to cardiomyopathic changes. Cardiovasc Res. 2012; 95:409-418.
25. Noorman M, Hakim S, Kessler E, Groeneweg J A, Cox M G, Asimaki A, van Rijen H V, van Stuijvenberg L, Chkourko H, van der Heyden M A, Vos M A, de Jonge N, van der Smagt J J, Dooijes D, Vink A, de Weger R A, Varro A, de Bakker J M, Saffitz J E, Hund T J, Mohler P J, Delmar M, Hauer R N, van Veen T A. Remodeling of the cardiac sodium channel, connexin43, and plakoglobin at the intercalated disk in patients with arrhythmogenic cardiomyopathy. Heart Rhythm. 2013; 10:412-419.
26. Gutman G A, Chandy K G, Grissmer S, Lazdunski M, McKinnon D, Pardo L A, Robertson G A, Rudy B, Sanguinetti M C, Stuhmer W, Wang X. International union of pharmacology. LIII. Nomenclature and molecular relationships of voltage-gated potassium channels. Pharmacol Rev. 2005; 57:473-508.
27. Garcia-Gras E, Lombardi R, Giocondo M J, Willerson J T, Schneider M D, Khoury D S, Marian A J. Suppression of canonical wnt/beta-catenin signaling by nuclear plakoglobin recapitulates phenotype of arrhythmogenic right ventricular cardiomyopathy. J Clin Invest. 2006; 116:2012-2021.
28. Ai Z, Fischer A, Spray D C, Brown A M, Fishman G I. Wnt-1 regulation of connexin43 in cardiac myocytes. J Clin Invest. 2000; 105:161-171.

TABLE 1
Changes in resting membrane potential, maximal upstroke velocity and
action potential duration in zebrafish ventricular myocytes expressing
2057del2 plakoglobin, and reversal by SB216763 at different ages
(weeks post-fertilization). Data are expressed as mean ± SE (n).

| | Weeks | | | | 2057del2 |
|---|---|---|---|---|---|
| | post | | 2057del2 | | plakoglo- |
| | fertili- | | plakoglo- | Control + | bin + |
| | zation | Control | bin | SB2 | SB2 |
| Resting | 3-4 | −78 ± | −67 ± | −79 ± | −75 ± |
| membrane | | 1 (8) | 2 (8)** | 1 (10) | 1 (7) |
| potential | 5-7 | −79 ± | −69 ± | −77 ± | −78 ± |
| (mV) | | 1 (11) | 1 (12)** | 1 (11) | 1 (11) |
| Maximal | 3-4 | 37 ± | 8 ± | 35 ± | 22 ± |
| upstroke | | 8 (8) | 1 (8)* | 8 (7) | 4 (5) |
| velocity | 5-7 | 63 ± | 18 ± | 53 ± | 66 ± |
| (V/s) | | 11 (10) | 2 (12)** | 11 (7) | 15 (8) |
| Action | 3-4 | 190 ± | 380 ± | 173 ± | 175 ± |
| potential | | 7 (8) | 38 (8)** | 8 (7) | 17 (5) |
| duration | 5-7 | 240 ± | 319 ± | 195 ± | 154 ± |
| (ms) | | 24 (11) | 44 (12)* | 17 (11) | 18 (11) |

*Significantly different than control (p < 0.05);
**significantly different than control (p < 0.001).
Zebrafish embryos at 1 dpf were treated with SB216763 for 6 days (3.0 μM) and then maintained under normal conditions (in the absence of SB2) until 3-4 or 5-7 weeks post-fertilization at which time ventricular myocytes were isolated and characterized. In other experiments, isolated ventricular myocytes were prepared from transgenic fish at 3-4 weeks post-fertilization and then treated in culture with SB216763 for 36 hr before being analyzed.

TABLE 2
Changes in I_{Na}, I_{K1} and I_{Kr}, in zebrafish ventricular myocytes express-
ing 2057del2 plakoglobin and reversal by SB216763 at different
ages (weeks post-fertilization). Data are expressed as mean ± SE (n).

| | Weeks | | | | 2057del2 |
|---|---|---|---|---|---|
| | post- | | 2057del2 | | plakoglo- |
| | fertili- | | plakoglo- | Control + | bin + |
| | zation | Control | bin | SB2 | SB2 |
| I_{Na}: peak | 3-4 | −212 ± | −51 ± 5 | −230 ± | −163 ± |
| current | | 19 (7) | (17)** | 27 (10) | 58 (7) |
| density | 7-8 | −196 ± | −31 ± | −168 ± | −172 ± |
| (pA/pF) | | 22 (18) | 3 (17)** | 39 (7) | 31 (8) |
| I_{K1}: current | 3-4 | −17 ± | −5 ± 0.8 | −18 ± | −15 ± |
| density at | | 2.7 (8) | (13)** | 3.5 (8) | 3.0 (8) |
| −100 mV | | | | | |
| (pA/pF) | | | | | |
| I_{K1}: slope be- | 3-4 | −27 ± | −8 ± 1.0 | −27 ± | −21 ± |
| tween −90 | | 4.3 (8) | (11)** | 5.0 (8) | 5.0 (6) |
| and −15 mV | | | | | |
| (pA/pF*100 | | | | | |
| mV) | | | | | |
| I_{Kr}: density of | 3-4 | 5.4 ± | 5.6 ± 0.7 | — | — |
| tail currents | | 0.8 (9) | (10) | | |
| at after acti- | | | | | |
| vation at +30 | | | | | |
| mV (pA/pF) | | | | | |

**Significantly different than control (p < 0.001).
Zebrafish embryos at 1 dpf were treated with SB216763 for 6 days (3.0 μM) and then maintained under normal conditions (in the absence of SB2) until 3-4 or 5-7 weeks post-fertilization at which time ventricular myocytes were isolated and characterized. In other experiments, isolated ventricular myocytes were prepared from transgenic fish at 3-4 weeks post-fertilization and then treated in culture with SB216763 for 36 hr before being analyzed.

### Example 4

### In Vivo Reversal of Arrhythmogenic Cardiomyopathy by SB216763

Arrhythmogenic cardiomyopathy (AC) is characterized by redistribution of junctional plakoglobin, gap junction remodeling, myocardial apoptosis, and arrhythmias. SB216763 (annotated as a GSK-31β blocker) reverses electrophysiological abnormalities in a zebrafish model of AC. It is demonstrated herein that SB216763 can rescue phenotypes in mouse models of AC that exhibit key features seen in patients.

2 different mouse models of AC were studied: a transgenic line with cardiac myocyte-specific expression of 2057del2 plakoglobin (Plk) and a mutant desmoglein-2 (Dsg2) knock-in model. ECG telemetry and echocardiographic analyses were performed in mutant and wildtype mice treated with daily injections of SB216763 or vehicle for 6 (2057del2 Plk) or 13 (Dsg2) weeks. Hearts excised at selected time points were analyzed for histology, apoptosis and distribution of key intercalated disk proteins.

Hearts of both mutant lines showed marked redistribution of junctional immunoreactive signals for Plk, Cx43 and SAP97, and increased myocardial apoptosis, all of which were normalized by SB216763 (data not shown). Plk mutant mice exhibited numerous PVCs (10.0±2.3/min) and short runs of VT, whereas SB216763-treated mice showed far fewer PVCs (2.8±0.6/min, p<0.05). Dsg2 mice showed a marked increase in QRS duration (from 16.9±2.7 to 17.6±3.1 ms), reflecting more ventricular ectopy, which was normalized by SB216763 (17.1±2.7; p<0.0001). Hearts of Plk mice showed myocyte necrosis, fibrosis and inflammation, which were nearly absent in mice treated with SB216763. Dsg2 mice showed reduced ejection fraction (44.5±11.8%) and fractional shortening (26.7±7.7%) compared to mutant mice treated with SB216763 (EF: 73.9±6.9%, p=0.035; FS: 50.2±6.6%, p=0.024).

The earliest evidence of disease in these mouse models (and in patients) is redistribution of Plk, Cx43 and SAP97. These changes precede (and, without wishing to be bound by theory, likely promote) arrhythmogenesis and occur prior to structural remodeling. During this early phase of AC, SB216763 can prevent both arrhythmias and myocardial lesions.

### Example 5

### An In-Vivo Drug Screen in Zebrafish Identifies a Novel Modulator of Intercalated Disc Remodeling in Arrhythmogenic Cardiomyopathy

Arrhythmogenic cardiomyopathy (ACM) is characterized by frequent arrhythmias often arising as the first manifestation of disease. To elucidate the underlying mechanisms and discover potential chemical modifiers, a zebrafish model of ACM with cardiac myocyte-specific expression of the human 2057del2 mutation in plakoglobin was created. A high-throughput chemical screen identified the putative GSK3β inhibitor SB216763 as a suppressor of the disease phenotype. Early SB216763 therapy prevented heart failure and reduced mortality in this fish model. Zebrafish ventricular myocytes expressing 2057del2 plakoglobin exhibited remodeling of action potentials with 70-80% reductions in INa and IK1 current densities, which were rapidly normalized by SB216763. Mammalian (neonatal rat) ventricular myocytes expressing 2057del2 plakoglobin recapitulated pathobiological features seen in patients with ACM including redistribution of plakoglobin from junctional to intracellular/nuclear sites, gap junction remodeling, cytokine expression and myocyte apoptosis, all of which were reversed or prevented by SB216763. The reverse remodeling observed with SB216763 involved marked subcellular redistribution of plakoglobin, connexin43 and Nav1.5 but without changes in their total cellular content, implicating a defect in protein trafficking to intercalated disks. In further support of this mechanism, abnormal subcellular distribution of SAP97 (a protein known to mediate forward trafficking of Nav1.5 and Kir2.1) was observed in cardiac myocytes expressing 2057del2 plakoglobin, which was reversed by SB216763. Similar findings were seen in cardiac myocytes derived from induced pluripotent stem cells from two ACM probands with plakophilin-2 mutations. Also demonstrated herein is abnormal SAP97 distribution in the myocardium of ACM patients. These observations identify aberrant trafficking of critical intercalated disk proteins as a central mechanism responsible for myocyte injury and electrical derangements in ACM.

Arrhythmogenic cardiomyopathy (ACM) is a highly proarrhythmic form of human heart disease and a significant cause of sudden death in the young (1, 2). First described as an isolated right ventricular disease (arrhythmogenic right ventricular cardiomyopathy or ARVC), ACM is now recognized to include biventricular and left dominant forms which may be misdiagnosed as dilated cardiomyopathy or myocarditis (3). Degeneration of cardiac myocytes and replacement by fibrofatty scar tissue develop with disease progression, but arrhythmias are often the earliest feature of ACM, and typically precede structural remodeling of the myocardium (1,2).

Mutations in several desmosomal proteins are known to cause ACM (1, 2). Abnormal localization of intercalated disk proteins including the desmosomal protein plakoglobin (γ-catenin), the gap junction protein Cx43 and the sodium channel protein Nav1.5 have been observed in patients with ACM and in experimental models (4-8). It has been proposed that ACM develops as a consequence of altered canonical Wnt signaling mediated by the mutant desmosomal proteins (9), but the extent to which ACM pathways perturb the constitution of junctional macromolecular assemblies is not known, and downstream mechanisms of myocyte injury and arrhythmogenesis remain obscure.

Homozygous null mutations in the genes that cause ACM are embryonic lethal in mice (10-12), and heterozygous null alleles for plakoglobin or plakophilin2 cause little if any phenotype (10, 12). Although heterozygous cardiac-restricted deficiency of desmoplakin expression causes ACM-like features (9), mechanisms of human germ-line mutant alleles remain unknown for all of the ACM genes described to date. Specific splice mutants have been transiently modeled using morpholinos in the zebrafish and the ability to identify developmental regulators and recapitulate the pathobiology of disease has been demonstrated (13,14). Now, to efficiently explore ACM pathophysiology, a stable inducible transgenic model capable of propagation through multiple generations and amenable to high-throughput genetic or chemical screening was developed. The GAL4/UAS transactivation system was used to drive cardiac myocyte-specific expression of the human 2057del2 mutation in the desmosomal protein plakoglobin (γ-catenin). This mutation causes Naxos disease in patients, a highly penetrant form of ACM associated with changes in hair and skin (15,16). Transgenic fish expressing this mutation develop abnormal cardiac physiology within 48 hours of fertilization with subsequent progression to a fully penetrant cardiomyopathy by 4-6 weeks characterized by cardiomegaly, cachexia, peripheral edema and, eventually, death due to heart failure or arrhythmia.

To optimize this line for high-throughput screening, a previously described natriuretic peptide b (nppb)::luciferase reporter line (17) was introduced onto the ACM mutant background. A library of bioactive compounds was screened for disease modifiers and 3 compounds that suppressed the disease phenotype were identified. One such suppressor, SB216763, previously annotated as an activator of canonical Wnt signaling (18), reduced nppb expression, prevented bradycardia and contractility defects and increased survival in mutant fish. Characterization of the electrophysiology of zebrafish ventricular myocytes and the pathobiology of neonatal rat ventricular myocytes expressing 2057del2 plakoglobin revealed that arrhythmias and myocyte injury in ACM are related to a common mechanism involving abnormal trafficking of key proteins to the intercalated disk mediated by the PDZ protein SAP97. Multiple, complex features of the disease phenotype in both fish and rat myocytes were rapidly corrected by SB216763. Similar findings were observed in cardiac myocytes derived from human induced pluripotent stem cells (hiPSCs) from 2 ACM disease probands with mutations in the gene encoding plakophilin-2. These observations indicate that ACM pathophysiology can be defined and disease modifying agents developed using SB216763 and its derivatives.

Results

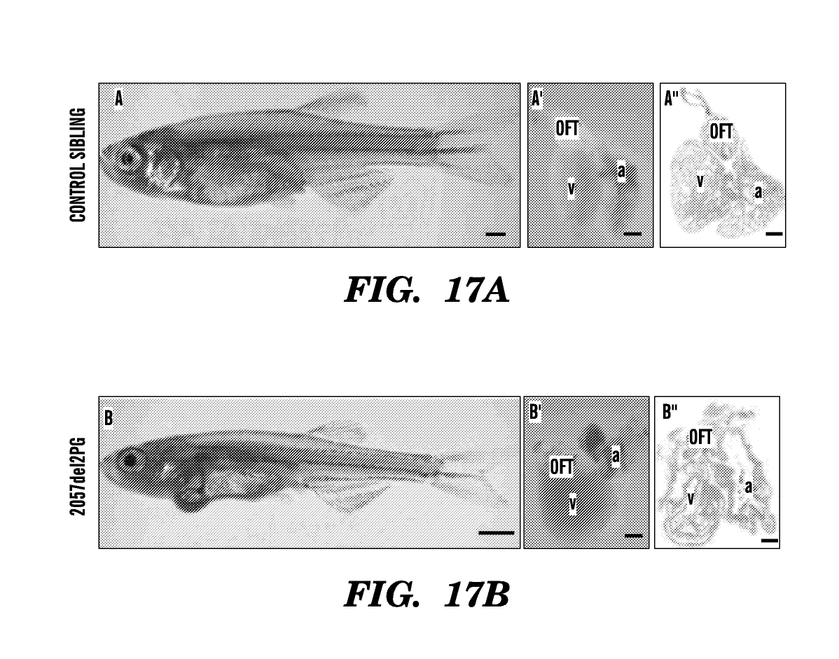

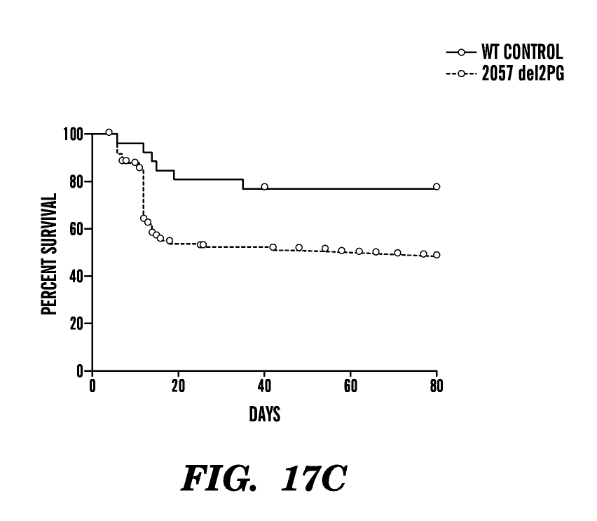

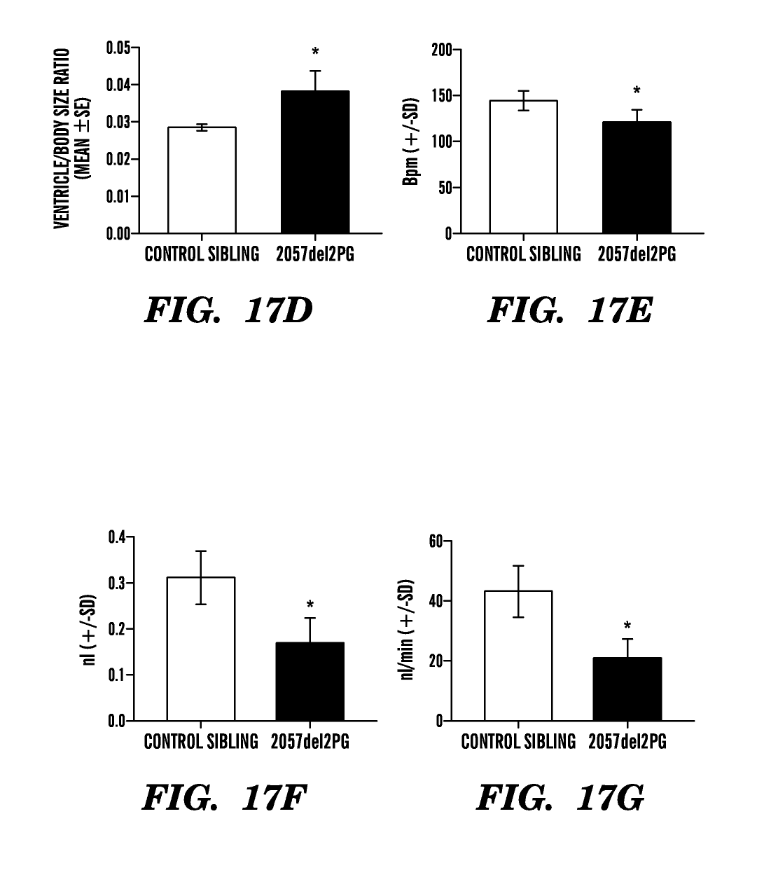

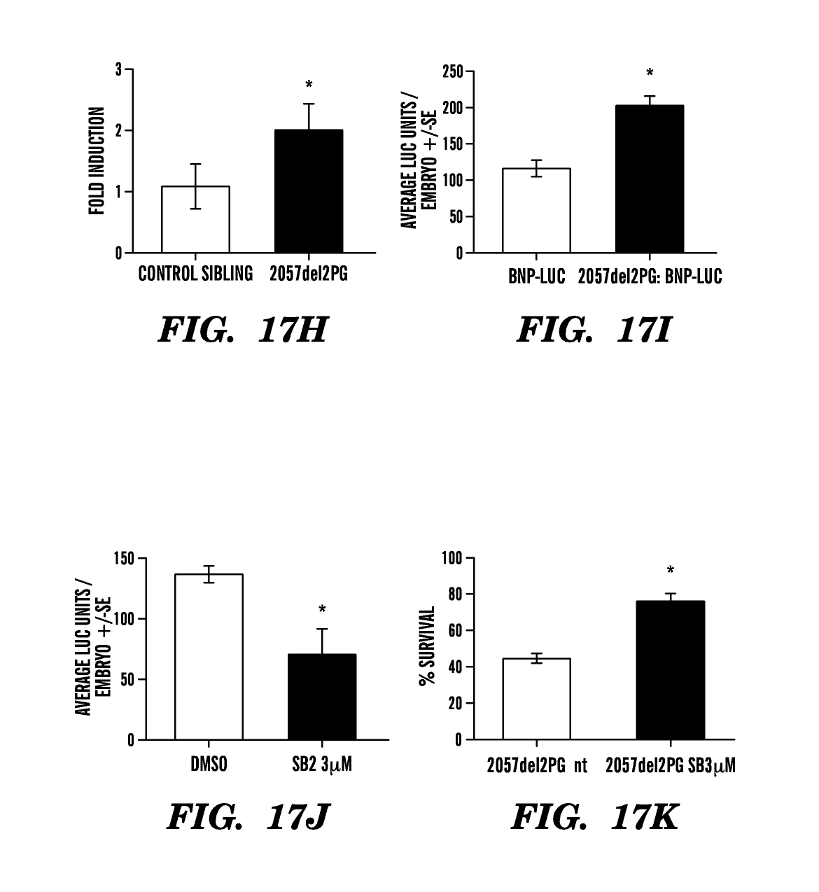

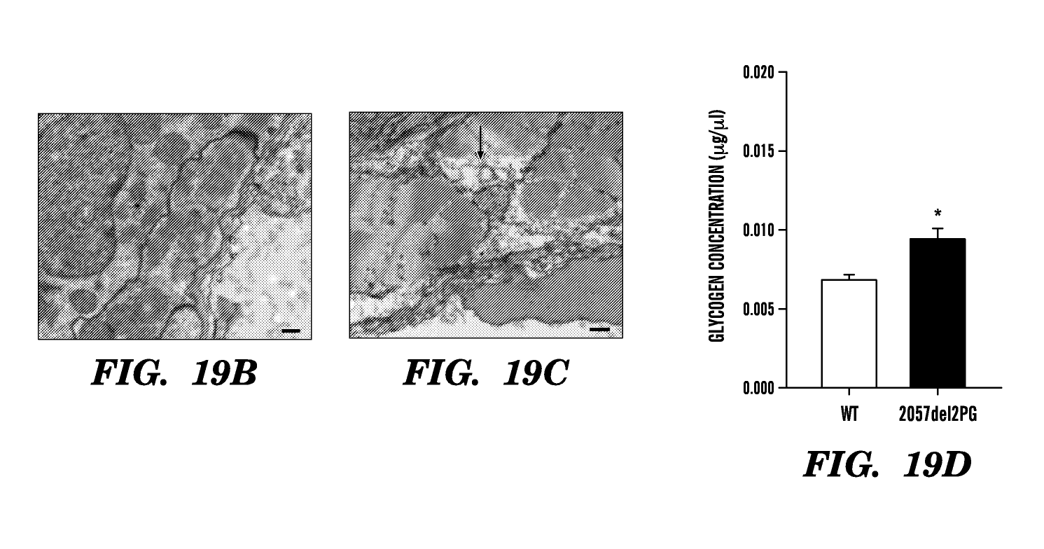

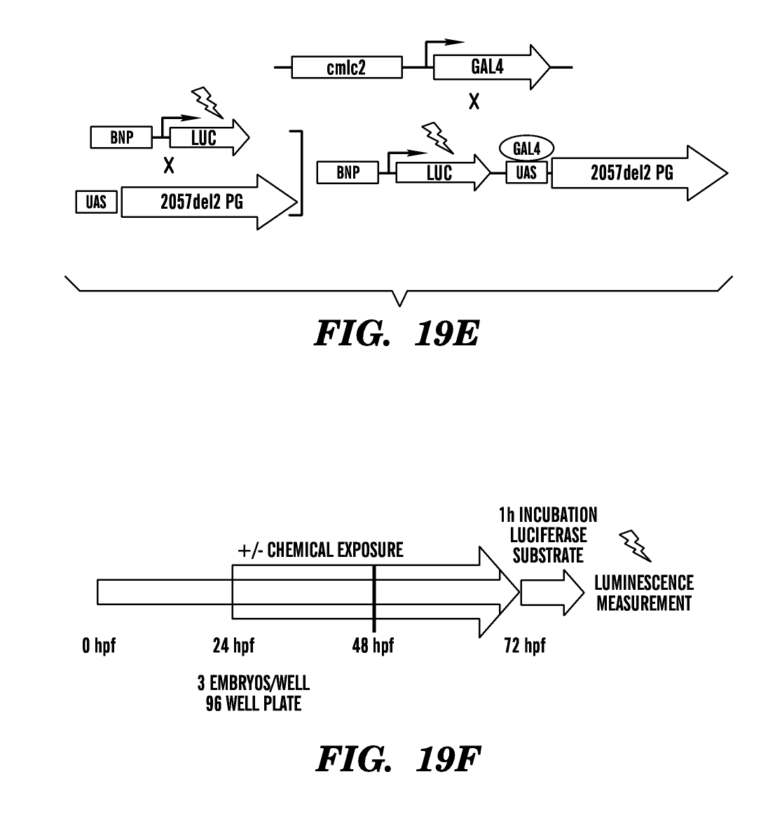

Generating a screenable zebrafish model of ACM: To study the effects of desmosomal mutations in a screenable system, the GAL4/UAS transactivation system was used to create zebrafish with cardiac myocyte-specific expression of the 2057del2 mutation in human plakoglobin (FIG. 19A). Cardiomegaly with marked thinning of atrial and ventricular walls, cachexia and peripheral edema were evident by early adulthood (5-6 weeks post-fertilization) (FIGS. 17A and 17B). Mutant fish exhibited substantial mortality (45% survival in mutants vs. 77% in controls, n=125; p<0.01) (FIG. 17C). The heart weight to body length ratio was increased at 3 months (FIG. 17D). Transmission electron microscopy (EM) showed interruptions in cell boundaries and structural disarray in mutant hearts compared to control siblings (FIGS. 19B and 19C). An increase in glycogen granules was seen on EM, and there was a significant increase in total myocardial glycogen content measured in the 2057del2 plakoglobin zebrafish model (FIG. 19D). To determine the feasibility of screening for a phenotype in 96 well plates at an early embryonic stage, the phenotypes were explored in larval mutant fish. By 48 hours post-fertilization (hpf) (late larval stage), mutant embryos exhibited a clear phenotype with mild bradycardia (144.2±10.8 beats/min in control vs. 120.8 ±12.6 in mutant, n=50; p<0.05), decreased stroke volume (0.31±0.06 nl in control vs. 0.17±0.05 in mutant, n=8; p<0.05) and reduced cardiac output (42.8 ±8.6 nl/min in control vs. 20.5±6.3 in mutant, p<0.05) (FIGS. 17E-17G).

Identification of disease modifiers in zebrafish via high-throughput chemical screening: To optimize this line for high-throughput screening, a previously described natriuretic peptide b (nppb)::luciferase reporter line (17) was introduced onto the ACM mutant background (FIG. 19E). Using qRT-PCR it was first demonstrated that the ACM mutant fish exhibited a significant induction (˜2 fold, p<0.01) of native nppb transcription at 48 hpf (FIG. 17H). It was confirmed that the nppb::luciferase reporter was also induced on the ACM background when crossed with the cmlc2::GAL4 driver fish (201.2±14.4 luciferase units per ACM mutant fish vs. 117.8±11.9 units per wildtype fish, n=30 fish in each group, p<0.01) (FIG. 17I). Once the baseline was defined for the larval model of ACM, a screen of a chemical library for modifiers of the nppb::luciferase phenotype (FIG. 19F) was begun. It was anticipated that toxic compounds would lead to very high or very low levels of nppb::luciferase activity (stress or death, respectively) depending on the relative timing of the drug effect with relationship to the assay schedule. The screen was therefor designed a priori to identify compounds that would normalize the npp::luciferase activity with tandem secondary screens that would confirm the effects of potential rescue compounds on cardiac physiology directly and also assess for more subtle forms of toxicity (14). To minimize false positives, assays were pre-specified in duplicate and only those compounds in which nppb::luciferase activity was within one standard deviation of the normal range in both instances were considered potential positives. This approach identified more than 50 first round ‘hits’ in a screen of 4,200 small molecules, all of which were followed up with additional testing in large numbers of embryos (n>50) for confirmation. Subsequent retesting and secondary assays restricted the initial number to 3 compounds of which SB216736 has the largest body of extant data (18). SB216736 at 3 μM in the well between 48 and 72 hpf normalized nppb::luciferase activity at 72 hpf (FIG. 17J) and longer term treatment of larval fish (7 days) led to substantially increased survival at 3 months (FIG. 17K). No developmental abnormalities were observed following SB216763 exposure at a wide range of doses during embryonic or larval development. Notably, no effect of SB216763 was detected on transcription of the plakoglobin transgene by reporter fluorescence of qRT-PCR (data not shown). In view of its prior annotation as a GSK-3β inhibitor (18), SB216736 was compared with other GSK-3β inhibitors (LiC1, CHIR99021, BIO (Sigma B 1686), SB415286 (Sigma S 3442), BIP 135 (Sigma SML0265)), none of which were as effective in attenuating the mutant phenotype (data not shown).

The effect of SB216763 on the cell biology of ACM was assesed. Cellular electrophysiology of zebrafish ventricular myocytes expressing 2057del2 plakoglobin: Marked changes in action potential morphology were observed in myocytes obtained from mutant fish at 5-7 weeks post-fertilization compared with myocytes from control fish (either wildtype or those with the mutant construct but without the GAL4 driver to elicit transgene expression) FIG. 9A-9B and Table 1). Resting membrane potential (RMP) was significantly depolarized in cells expressing 2057del2 plakoglobin compared with controls (−69±1 mV vs. −79±1 mV, respectively, n=12 or 11; p<0.001). The maximum rate of rise in voltage during phase 0 of the action potential, dV/dtmax, was markedly blunted (18±2 V/sec vs. 63±11 V/sec, n=12 or 10; p<0.001), and action potential duration at 80% completion of repolarization (APD80%) was prolonged (319±44 ms vs. 240±24 ms, n=12 or 11; p<0.05). Nearly identical changes in action potential shape were observed in myocytes isolated from fish 3-4 weeks post-fertilization, indicating no major progression in the action potential phenotype between 3 and 7 weeks of age (Table 1). Furthermore, virtually identical changes in action potential morphology (positive shift in RMP, reduction of dV/dtmax and prolongation of APD80%) were observed in neonatal rat ventricular myocytes expressing 2057del2 plakoglobin (FIG. 9C). These observations indicate that the action potential phenotype observed in the fish model reflects critical features of disease exhibited by mammalian ventricular myocytes expressing a known human ACM disease gene, and presumably, by ventricular myocytes in patients with ACM.

The marked decrement in dV/dtmax in phase 0 of the action potential prompted us to characterize potential changes in INa in zebrafish ventricular myocytes expressing 2057del2 plakoglobin. Using whole-cell voltage-clamp and starting from a holding potential of −80 mV, we observed a large reduction in INa current density across a broad range of membrane potentials (FIGS. 10A-10B and Table 2). Peak Ina current density was reduced by 84%, from 196±22 pA/pF in control cells (n=18) to 31±3 pA/pF (n=16; p <0.001) in myocytes expressing 2057del2 plakoglobin isolated from fish at 5-7 weeks post-fertilization. Similar reductions (76%) were observed in myocytes isolated from fish at 3-4 weeks post-fertilization (Table 2). No significant shift was seen in steady-state activation and inactivation curves of INa (FIG. 10C). Thus, the marked reduction in INa current density in fish myocytes expressing 2057del2 plakoglobin appears to be due solely to a reduction in the number of functional channels at the sarcolemma.

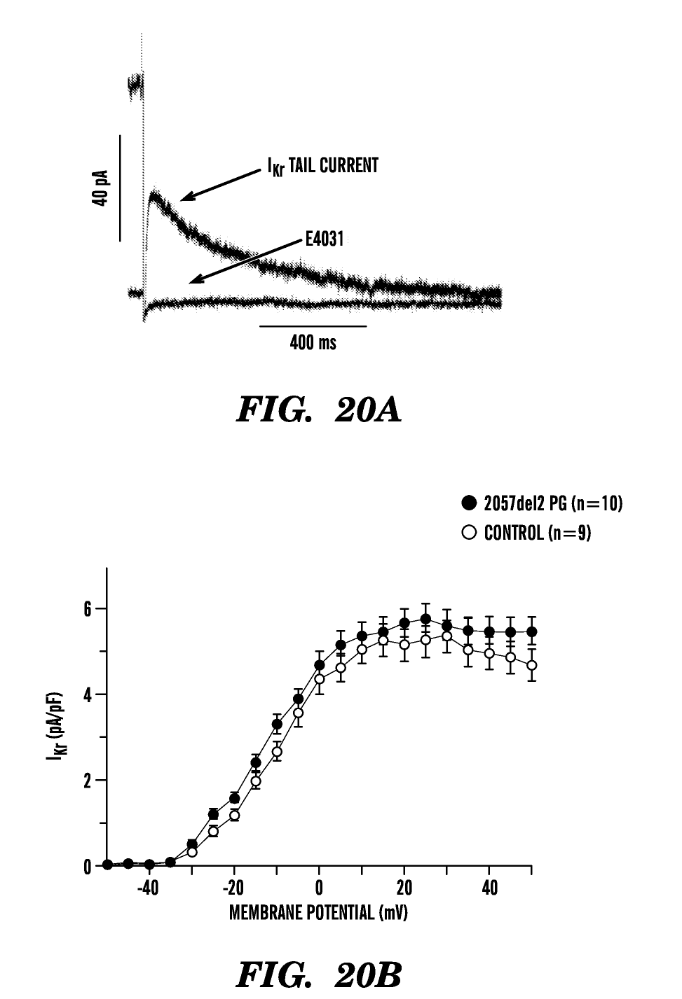

The more depolarized resting membrane potential in myocytes expressing mutant plakoglobin raised the possibility of alterations in IK1, a current known to be important in maintaining normal resting potential. Indeed, a similar marked decrease (71%) was observed in IK1 current density at −100 mV (from 17.0±2.7 pA/pF to 5±0.8 pA/pF, n=8 and 13 respectively; p<0.001) in cells expressing 2057del2 plakoglobin (FIGS. 10D-10F and Table 2). However, no difference was observed in IKr current density between control myocytes and myocytes expressing 2057del2 plakoglobin (FIG. 20A-20B and Table 2).

Reversal of electrophysiological derangements by SB216763 in cells expressing 2057del2 plakoglobin: To determine whether SB216763 affects action potential remodeling and changes in INa and IK1 observed in zebrafish ventricular myocytes expressing 2057del2 plakoglobin, cellular electrophysiology was characterized in isolated myocytes exposed to SB216763 at two different developmental stages. In these experiments, a similar concentration of SB216763 (5 μM) found previously in the chemical screen to reverse the disease phenotype was used. Vehicle controls (DMSO) were also analyzed and showed no effect. In the first protocol, zebrafish embryos at 1 dpf were treated with SB216763 for 6 days and then maintained under normal conditions (in the absence of SB216763) until 5-7 weeks post-fertilization at which time ventricular myocytes were isolated and studied by current-clamp and whole-cell voltage-clamp methods. As shown in Table 1, SB216763 did not affect action potentials in myocytes from control fish, whereas it normalized RMP and dV/dtmax, and actually shortened APD80% beyond control levels in fish expressing mutant plakoglobin.

Similarly, early exposure to SB216763 had no effect on INa or IK1 current densities in control myocytes but it almost completely normalized these currents in cells expressing 2057del2 plakoglobin (Table 2). Thus, transient exposure to SB216763 during embryonic and larval development durably prevents action potential remodeling in fish expressing mutant plakoglobin even as adults. To determine whether SB216763 can reverse these changes once they occur, a second protocol was performed in which isolated ventricular myocytes were prepared from transgenic fish at 5-7 weeks post-fertilization (when the action potential phenotype is fully developed) and then treated in culture with SB216763 for 36 hr before being analyzed. As shown in FIG. 12 and Tables 1 and 2, short-term exposure of isolated cells to SB216763 normalized action potential remodeling and reversed reductions in INa and IK1 current densities. The fact that these changes can be normalized in isolated cells in vitro indicates that the marked cardiac myocyte electrical phenotype produced by expression of an ACM disease gene is cell autonomous. Moreover, the electrical phenotype can apparently be prevented from occurring in adult fish when early embryos are transiently exposed to SB216763, and once it becomes fully developed in adult fish, it can be reversed by briefly exposing cells to SB216763.

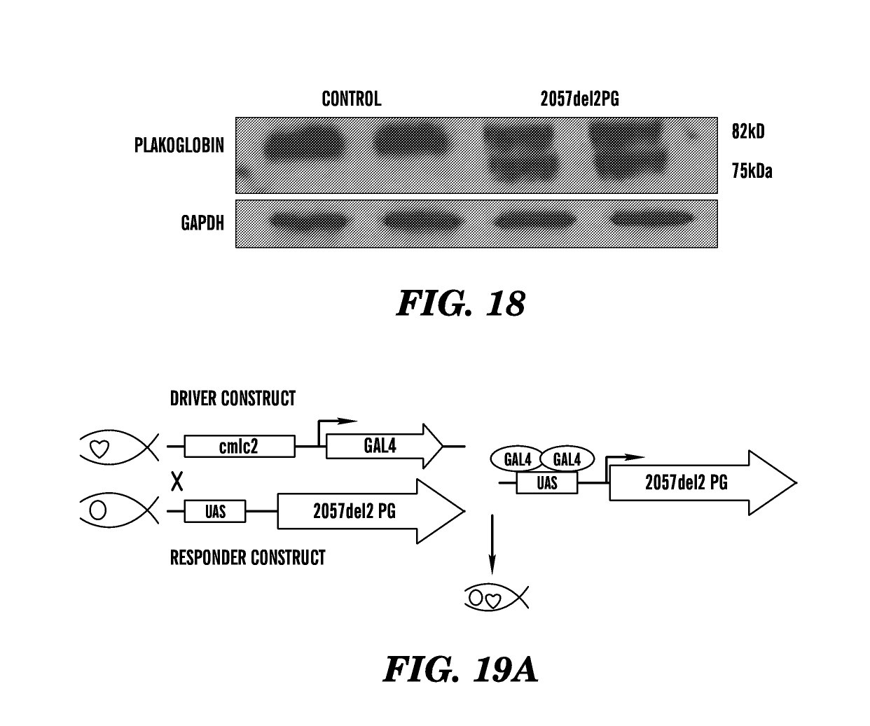

An in vitro mammalian model recapitulates cellular features of ACM: Previous studies of human myocardium have identified 4 features that appear to play a role in the pathogenesis of ACM in patients. These include: 1) decreased immunoreactive signal for plakoglobin at cell-cell junctions (4); 2) gap junction remodeling indicated by decreased immunoreactive signal for the major ventricular gap junction protein, Cx43, at cell-cell junctions (4); 3) myocardial apoptosis (19); and 4) high circulating levels of pro-inflammatory cytokines and expression of cytokines by cardiac myocytes (20). To gain insights into mechanisms responsible for these features of ACM in patients, an in vitro model was developed in which normal neonatal rat ventricular myocytes were transfected with adenovirus to express 2057del2 plakoglobin containing a V5 epitope tag. Conditions yielding >95% transfection (assessed using adenovirus containing GFP) and expression of 2057del2 plakoglobin at levels roughly equivalent to that of the endogenous normal protein (FIG. 18) were identified. The effects of transgene expression on development of features identified in the human disease were then assessed. As illustrated in FIG. 8D, expression of 2057del2 plakoglobin for 24 hr led to 1) a marked change in the distribution of immunoreactive signal for plakoglobin with diminished signal at cell-cell junctions and abundant signal in nuclei; 2) greatly reduced immunoreactive signal for Cx43 at cell-cell junctions; and 3) increased myocyte apoptosis indicated by greater numbers of TUNEL-positive nuclei and increased expression of caspase-3. Apoptosis in cells expressing 2057del2 plakoglobin could be prevented by pifithrin-A (5 μM) which inhibits p53 transcriptional activity, or greatly increased by subjecting cells to brief intervals of uniaxial cyclical stretch (FIG. 8B). Finally, cells transfected to express 2057del2 plakoglobin for 24 hr secreted various inflammatory mediators into the culture medium including IL-6, TNFα, MIPla, RANTES, and IL-17 (FIG. 8C), several of which have been identified in the blood or myocardium in patients with ACM (20). Taken together, these results indicate that expression of 2057del2 plakoglobin by neonatal rat ventricular myocytes recapitulates features consistently identified in the hearts of patients with ACM.

Reversal of disease features by SB216763 in mammalian myocytes expressing 2057del2 plakoglobin: To determine whether SB216763 is capable of mitigating the effects of 2057del2 plakoglobin expression in mammalian cardiac myocytes, cells were transfected and 24 hr later exposed to SB216763. A dose-response study was performed in which neonatal rat ventricular myocytes expressing mutant plakoglobin were exposed to SB216763 (0.1-10 μM) for 12 or 24 hours. The maximal effect in restoring the normal cellular distribution of plakoglobin and Cx43 immunoreactive signals was observed at a concentration of 5 μM for 24 hr (data not shown). Thus, all subsequent experiments were performed using these conditions. Vehicle controls (DMSO) were also analyzed and showed no effect. SB216763 prevented the marked change in subcellular distribution of plakoglobin and accumulation of nuclear signal seen in cells expressing mutant plakoglobin. It also prevented the adverse remodeling of gap junctions as indicated by the presence of control levels of junctional Cx43 signal in treated cells, and it increased the amount of Cx43 signal at cell-cell junctions in control (non-transfected) cultures (data not shown). Most of the Cx43 signal in control cells and in SB216763-treated cells expressing 2057del2 plakoglobin was localized at the cell surface as shown by double-label (Cx43 and N-cadherin) immunohistochemistry (data not shown). The marked changes in the distribution of immunoreactive signals for plakoglobin and Cx43 and the nearly complete reversal of these changes in cells expressing 2507del2 plakoglobin occurred with no apparent change in the total cellular content of plakoglobin and a modest increase in the total cellular content of Cx43 (both in control and transfected myocytes) as assessed by immunoblotting (FIG. 11A). These observations, coupled with the temporal course with which these proteins changed their subcellular distribution, suggest that 2057del2 disturbs the normal trafficking of plakoglobin and Cx43 to the cell surface, and that SB216763 corrects this defect. SB216763 also dramatically reduced the number of TUNEL positive nuclei in myocytes expressing mutant plakoglobin (FIG. 11B). Finally, there was no apparent effect on cytokines accumulating in the culture medium in transfected cells incubated with SB216763 for 24 hr (data not shown), but there was a clear reduction in cytokine levels in media recovered from cells exposed to 5 μM SB216763 for 48 hr (FIG. 11C). An even greater diversity of cytokines was identified in the culture medium from cells expressing 2057del2 plakoglobin for 48 vs. 24 hr (compare FIGS. 8C and 11C), but in each case these were all reduced by SB216763. Taken together, these observations indicate that changes in the distribution of critical junctional proteins, expression of inflammatory markers of cell injury and apoptosis of cardiac myocytes are all mediated by a common disease pathway that can be ameliorated by SB216763.

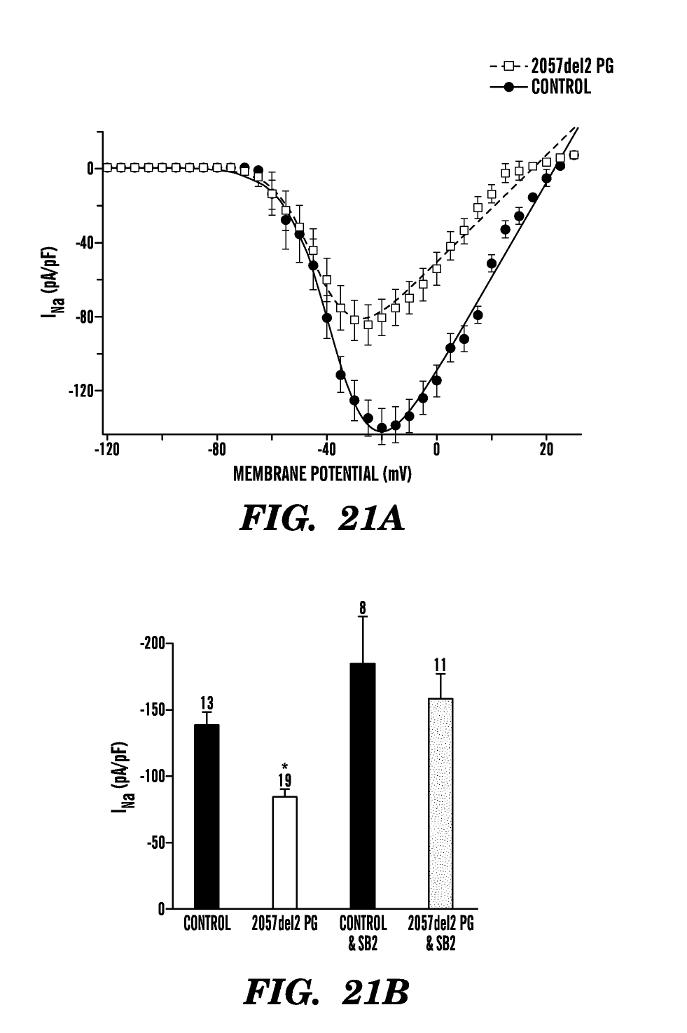

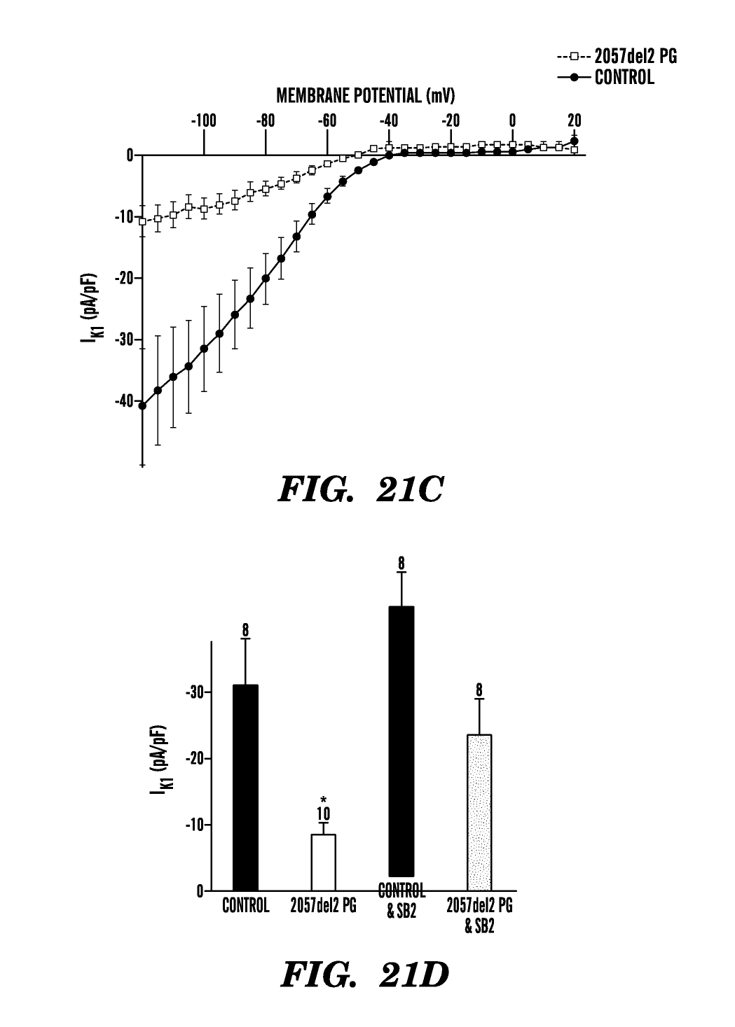

To determine if the cellular electrophysiology phenotype seen in zebrafish myocytes expressing 2057del2 plakoglobin also occurs in mammalian myocytes expressing the same mutant protein, INa and IK1 current density were measured across a broad range of membrane potentials in isolated neonatal rat ventricular myocytes. As shown in FIGS. 21A-21D, significant reductions in INa and IK1 were seen in myocytes expressing 2057del2 plakoglobin compared to control cells. And, SB216763 normalized the reduced current densities in cells expressing mutant plakoglobin (FIGS. 21A-21D).

SB216763 is annotated as a GSK-3β inhibitor (18). To determine if other such blockers exert similar effects, neonatal rat ventricular myocytes expressing 2057del2 plakoglobin were exposed to SB415286 or CHIR99021 (at 5 μM for 24 hr). SB415286 increased plakoglobin signal at cell-cell junctions and reduced nuclear signal but had no effect on Cx43 signal, whereas CHIR99021 apparently intensified plakoglobin signal in nuclei (data not shown). Thus, despite being similarly annotated as GSK-3β inhibitors and as seen in the zebrafish model, neither SB415286 nor CHIR99021 was as effective as SB216763 in reversing the disease phenotype in mammalian myocytes expressing 2057del2 plakoglobin.

Effects of 2057del2 plakoglobin on expression and distribution of SAP97 and Nav1.5: The PDZ protein SAP97 has been shown to regulate normal targeting to the cell surface of Nav1.5 and Kir2.1 (21), the major protein subunits responsible for INa and IK1, respectively. To determine whether derangements in this trafficking pathway might account for the rapidly reversible reductions in INa and IK1 observed in myocytes expressing mutant plakoglobin, immunohistochemistry and immuno-blotting were used to characterize the distribution and total cellular content of SAP97 in neonatal rat ventricular myocytes transfected to express 2057del2 plakoglobin. As shown in FIG. 13, immunoreactive signal for SAP97 was concentrated at the cell surface in control myocytes, similar to the previously reported localization of this protein to intercalated disks in adult rat ventricular myocardium (22). However, there was a marked decrease in the amount of SAP97 signal at the cell surface in myocytes expressing 2057del2 plakoglobin, which was fully reversed by exposing myocytes to SB216763 for 24 hr (FIG. 13). These rapidly reversible changes in subcellular distribution of SAP97 immunoreactive signal occurred without apparent changes in the total amount of SAP97 within the myocytes as shown by Western blots (FIG. 13). Taken together with the previous electrophysiological studies, these results suggest that defective trafficking of ion channel proteins is the mechanism underlying action potential remodeling and reduced altered INa and IK1 current densities in ventricular myocytes expressing 2057del2 plakoglobin. To provide additional independent evidence in support of this mechanism, the distribution and total cellular content of Nav1.5 in neonatal rat myocytes was characterized. As shown in FIG. 13, there was a marked reduction in immunoreactive signal for Nav1.5 in myocytes expressing mutant plakoglobin, and this was reversed in cells exposed to SB216763 for 24 hr. Nav1.5 signal appeared to be distributed both intracellularly and at the cell surface in control neonatal rat myocytes and in SB216763-treated myocytes expressing 2057del2 plakoglobin. Attempts to quantify the amount of Nav1.5 signal at the cell surface using double-label immunohistochemistry were technically difficult because of high background signal. However, the marked reduction in INa in zebrafish myocytes expressing mutant plakoglobin and its rapid, near total recovery after exposure to SB2168763 indicate a similar reversible loss of cell surface Nav1.5 in mammalian myocytes expressing 2057del2 plakoglobin.

Despite the marked changes in the apparent distribution of Navl 5 immunoreactive signal in neonatal rat ventricular myocytes, there was no difference in the total content of Nav1.5 protein assessed by immunoblotting in control cells or cells expressing mutant plakoglobin (FIG. 13). These observations provide additional independent evidence that changes in cellular electrophysiology in ACM are related to defective forward trafficking of key ion channel proteins rather than to insufficient channel protein production. They also indicate a common disease pathway, sensitive to the mitigating effects of SB216763, underlying both myocyte injury/apoptosis and arrhythmias in ACM.

To further investigate the role of SAP97 in ACM, shRNA was used to knock-down SAP97 expression in normal neonatal rat ventricular myocytes and characterized the effects on the distribution of key intercalated disk proteins. Knock-down of SAP97 expression (demonstrated by greatly reduced SAP97 immunosignal in treated cells) led to marked reduction in immunosignal not only for Nav1.5, as previously shown (21), but also for plakoglobin (data not shown). There was no effect, however, on the distribution of signals for other desmosomal proteins such as desmoplakin or plakophilin-2. This suggests a unique relationship between plakoglobin and SAP97 not shared by other desmosomal linker proteins. Finally, knock-down of SAP97 expression had no effect on the distribution of Cx43, whose trafficking is known to involve a different transport mechanism (22).

Abnormal distribution of SAP97 in myocardium from patients with ACM: To determine whether insights into disease mechanisms gained from analysis of zebrafish myocytes and confirmed in mammalian myocytes in vitro apply to the actual human disease, sections of formalin-fixed paraffin-embedded myocardium from patients with ACM using were immunostained anti-SAP97 antibodies. Tissue was analyzed from four control subjects (non-cardiac deaths) and eight patients with documented ACM including two each with mutations in plakophilin-2 or desmoplakin, one each with mutations in plakoglobin or desmoglein2, and two patients with clinical expression of disease but no apparent desmosomal gene mutation. Control human myocardium showed immunoreactive signal concentrated at intercalated disks and in a sarcomeric pattern (data not shown), identical to that shown previously in adult rat ventricular myocardium (21). By contrast, a marked reduction in SAP97 signal was seen in the ventricular myocardium of patients with ACM independent of the specific mutation involved in causing their disease (attempts to analyze the distribution of Nav1.5 in patient material were unsuccessful because available antibodies did not work in formalin-fixed paraffin-embedded tissues). To determine whether reduced SAP97 signal is specific for ACM, 15 myocardial samples from patients with end-stage ischemic, dilated or hypertrophic cardiomyopathy were stained. As seen The myocardium in these other forms of human heart disease showed near-control levels of SAP97 signal in a sarcomeric distribution (although there did seem to be modest relative loss of signal at intercalated disks) (data not shown0. Thus, SAP97 immunoreactive signal seems to be preferentially decreased in the hearts of patients with ACM compared to other forms of heart disease. Recognizing the limitations of immunofluorescence in formalin-fixed, paraffin-embedded sections of human myocardium, additional studies were performed in cardiac myocytes derived from human induced pluripotent stem cells (hiPSCs) generated from peripheral blood mononuclear cells obtained from two ACM probands each with distinct truncating mutations in plakophilin-2 (Q617X and 2013delC). Cardiac myocytes derived from hiPSCs of unaffected (mutation-negative) siblings of each proband were used as controls. Methods of blood cell reprogramming and cardiac myocyte differentiation are included in supplemental materials. Cardiac myocytes from probands in both families exhibited marked reduction in immunoreactive signals for plakoglobin, Cx43, SAP97 and Nav1.5 when compared to cardiac myocytes from unaffected siblings (data not shown). These abnormalities were reversed in both proband myocyte lines after exposing cells to SB216763 for 24 hr. Taken together with observations in patient myocardium (data not shown), these results further implicate deranged SAP97 distribution in ACM, validate the present observations in both fish and neonatal rat models of ACM and indicate that SAP97 is a biomarker of disease severity and arrhythmogenesis in ACM.

Discussion:

ACM is a deadly disease for which no mechanism-based therapies exist (1,2). To understand why it is so arrhythmogenic and to identify potential drug candidates, a screenable model of ACM in zebrafish was developed and a small molecule that rescues the disease phenotype and reduces mortality identified. Also described herein are marked abnormalities in action potentials and ionic currents in fish ventricular myocytes that likely contribute to arrhythmogenesis in ACM. Armed with information from studies in fish, these observations were confirmed and extended in studies of mammalian ventricular myocytes to show that myocyte injury and arrhythmogenesis are linked to a common disease pathway. Insights gained from studies in fish and rat myocytes ultimately led to identification of SAP97 as a biomarker in ACM patients that appears to be part of this disease pathway.

The genetics of ACM are complex. Most cases are autosomal dominant and many are a consequence of mutations in desmosomal protein genes (1-3). However, penetrance is usually highly variable and disease expression can vary widely even within individual families. Recessive forms of ACM exist for some disease genes. In such cases, the phenotype may be more severe, more fully penetrant, and include hair and skin components. Importantly, true null alleles in these genes are not the cause of ACM in patients. Available evidence indicates that mutant proteins are expressed in patients with ACM, and multiple murine models show that homozygous null alleles in these same genes are embryonic lethal (10-12). This is certainly the case in Naxos disease in which the mutant gene product is abundantly expressed in the myocardium (23). To allow in vivo screening at scale, a bona fide human allele was modeled in transgenic form. This sensitized system not only faithfully recapitulates key features of the human disease both in the zebrafish and in mammalian cells, it offers proof-of-concept for chemical suppression of a structurally abnormal junctional protein. Furthermore, salient features of the disease phenotype seen in fish and rat myocytes expressing 2057del2 plakoglobin were also seen in human cardiac myocytes derived from hiPSCs from two ACM probands with different mutations in plakophilin-2.

Normal neonatal rat ventricular myocytes respond to brief intervals of cyclical stretch by rapidly increasing levels of immunoreactive signals for proteins at cell-cell junctions including plakoglobin and Cx43 (24). By contrast, myocytes expressing 2057del2 plakoglobin fail to increase cell-cell junction proteins in response to stretch. Rather, they exhibit greatly increased rates of apoptosis after being stretched, whereas control cells show no appreciable apoptosis. These observations are consistent with clinical studies showing that exercise increases age-related penetrance and arrhythmic risk in ACM patients (25). They also indicate that zebrafish and mammalian myocyte models involving transgenic expression of 2057del2 plakoglobin are useful in studies to define mechanisms responsible for exercise-induced disease exacerbations in ACM patients.

Reduced INa current density has been observed previously in neonatal rat myocytes following knock-down of plakophilin-2 (6) and in a transgenic mouse model with cardiac myocyte expression of mutant desmoglein-2 (7). The present observations provide evidence in both fish and neonatal rat myocyte models that reduced INa current density is attributable largely, if not entirely, to a reduced number of functional channels at the cell surface rather than to changes in activation/inactivation kinetics. This conclusion is supported by reduced immunosignal for Nav1.5 in neonatal rat myocytes expressing mutant plakoglobin, an observation we have also reported in patients with ACM (8). The finding, described herein, of reduced IKlcurrent density not only implicates another potentially highly arrhythmogenic electrophysiological defect in ACM, but also led to discovery of aberrant SAP97 distribution in myocytes expressing mutant plakoglobin and in ACM patients whose disease is linked to various mutations in desmosomal genes. This observation is particularly intriguing because SAP97 has been shown to associate with Nav1.5 (26) and to regulate forward trafficking of Nav1.5 and Kir2.1 to the cell surface (21). These proteins form channels responsible for INa and IK1, respectively, both of which were found were dramatically reduced in fish and neonatal rat myocytes expressing 2057del2 plakoglobin. No such reduction was seen, however, in IKr current density, a channel not associated with SAP97 (27). An unanticipated observation was abnormal distribution of plakoglobin (but not other desmosomal linker proteins, N-cadherin or Cx43) in normal neonatal rat myocytes after knock-down of SAP97 expression. Taken together, these observations strongly implicate altered trafficking of Nav1.5, Kir2.1 and plakoglobin (and potentially other proteins complexed with them) as a critical component of the disease pathway in ACM.

SB216763 exhibits a remarkable ability to prevent or reverse features of the disease phenotype in both zebrafish and rat models of ACM induced by expression of 2057del2 plakoglobin. SB216763 is thought to activate the canonical Wnt signaling pathway by inhibiting GSK-3β (18). Increasing evidence from mouse models of ACM has implicated changes in Wnt signaling in disease pathogenesis (9), but the underlying mechanisms are poorly understood and there has been little if any direct evidence to prove a causal relationship. Increased Cx43 expression was not observed in both control myocytes and 2057del2 plakoglobin-expressing cells exposed to SB216763, consistent with previous studies showing that Cx43 gene expression can be up-regulated by activation of the canonical Wnt signaling pathway (28). These observations provide strong evidence for a unifying trafficking abnormality in ACM that can account for diverse features of the disease including loss of plakoglobin from junctions, remodeling of gap junctions, myocyte injury/apoptosis and action potential remodeling. Perhaps most remarkably, it was observed that transient exposure to SB216763 of early zebrafish embryos undergoing cardiogenesis produces lasting effects to prevent action potential remodeling. Excessive mortality in fish with cardiac myocyte-specific expression of 2057del2 plakoglobin occurs within the first 10 days of development and subsequently levels off thereafter to control rates (FIG. 17C). This implies an early effect of the disease pathway during maturation, which, if blocked at a critical time, might prevent subsequent action potential remodeling. Yet, even after becoming fully manifest, action potential remodeling can still be rescued by SB216763, indicating ongoing reversibility of the effects of the common disease pathway.

Materials and Methods

Rationale and design of study: The overall objective of this study was to create a model of ACM in zebrafish to facilitate drug discovery via high-throughput chemical screening. Having identified a small molecule that rescues the phenotype in fish, characterization of electrophysio-logical and cell injury phenotypes in zebrafish and neonatal rat ventricular myocyte models was preformed, demonstrating that they exhibit key features of the disease seen in patients and, second, that both the electrophysiological and cell injury phenotypes can be reversed by the drug candidate identified in the chemical screen. Finally, insights gained through this approach were applied to studies of diseased myocardium and iPSC-derived cardiac myocytes from ACM patients to implicate aberrant trafficking of critical intercalated disk proteins as a central mechanism responsible for myocyte injury and electrical derangements in ACM.

Zebrafish care and embryo collection: All experiments were performed in Tuebingen AB zebrafish or Danio fish obtained from Ekkwill Waterlife Resources (Ruskin, Fla.). Zebrafish were bred and maintained at 28.5° C. with 14 hr light and 10 hr dark exposure (day/night cycle) according to protocols approved by the Beth Israel Deaconess Medical Center IACUC and the Harvard Subcommittee for Animal Research. The stages (hours post-fertilization, hpf) described in this report are based on the developmental stages of normal zebrafish embryos at 28.5° C. (29).

Cloning and transgenic fish generation: The UAS/2057del2 plakoglobin responder construct was created using the Gateway cloning system (Invitrogen, Carlsbad, Calif., USA) and the Michael L. Nonet laboratory (Washington University, St. Louis, Mo., USA) pBH (bleeding heart) UAS vector (FIG. 19A). The destination vector was modified by removing the YFP sequence and replacing the cmle2 promoter with the Xenopus crystalline promoter which drives expression in zebrafish lens, allowing efficient identification of the transgenic line in sub-sequential generations. Gateway recombination reactions were performed according to the manufacturer's instructions (Invitrogen). Single cell embryos were injected with 15 ng/nl destination vector DNA and 15 ng/nl of Tol2 transposase RNA. Stable transgenics were selected and out-bred for multiple generations.

Zebrafish cardiac physiology: Images of live zebrafish hearts were acquired on an Axioplan™ (Zeiss) upright microscope with a 5× objective lens using integrated incandescent illumination and a FastCam-PCI™ high-speed digital camera (Photron USA) with 512×480 pixel grey-scale image sensor. Images were obtained sequentially at 250 frames per second, with 1088 frames (approximately 8 cardiac cycles) acquired per condition. In-house software (implemented in MATLAB™) was used to determine heart rate, while measurements of ventricular long and short axes in both diastole and systole were obtained using ImageJ™. Cardiac output was measured as diastolic minus systolic ventricular volume multiplied by heart rate, as outlined by Shin et al (30). Fractional shortening was calculated as end-diastolic diameter minus end-systolic diameter divided by end-diastolic diameter.

Chemical screening: Chemicals were dissolved in DMSO (Sigma-Aldrich, St. Louis, Mo., USA). Embryos (double homozygous for the 2057del2 plakoglobin mutation and the nppb::luciferase reporter) were arrayed in 96 well plates in HEPES-buffered E3 medium and small molecules were pin-transferred (200 n1) from arrayed chemical libraries. Positive (untreated wild type nppb::luciferase embryos) and negative controls (untreated 2057del2 plakoglobin/nppb::luciferase fish) were included in each plate. Compounds were added at 24 hpf and assays were performed at 72 hpf (FIG. 19F). Each set of compounds was tested in duplicate to improve the discriminatory power of the assay. After being evaluated for viability, an equal volume of long half-life luciferase reagent (Promega Steady Glo™) was added to each well. The plate was then incubated in the dark for 60 min and activities were measured with a Victor 3™ luminometer (Perkin-Elmer). Each measurement was performed in duplicate and results were normalized to the number of fish in the well. Follow-up chemical treatments were performed in HEPES-buffered E3 medium in petri dishes.

Survival studies: Embryos were kept in petri dishes until 7 dpf and then transferred to nursery tanks with larval food. Observations for phenotype and survival were recorded for several weeks. Data are presented as a Kaplan-Meier curve using Graphpad™ software.

Zebrafish ventricular myocyte isolation and culture: Hearts were extracted from anesthetized fish at 3-4 or 5-7 weeks post-fertilization using micro-dissecting tweezers (Roboz Surgical Instruments), washed vigorously in culture medium (M199, primocin, HEPES, L-glutamine, 10% fetal bovine serum) and placed in fresh medium. Ventricles were separated at the atrio-ventricular junction, pooled in a tube containing enzyme solution (calcium/magnesium free-HBSS containing trypsin, penicillin/streptomycin, HEPES) at 37° C. and agitated in a thermomixer at 1000-1200 RPM. Dissociated cells were collected every 10 min from supernatants and transferred to tubes containing culture medium and 10% fetal bovine serum to halt trypsinization. Cells were then pelleted by centrifugation, resuspended in fresh culture medium, and seeded on sterile cover slips coated with laminin (20 μg/ml) at 28.5° C. in a 5% CO2 incubator. After 36 hr, culture medium was changed and unattached cells were removed.

Cellular electrophysiology methods: Current clamp experiments in isolated neonatal rat and zebrafish ventricular myocytes were carried out using standard protocols (31). Micropipettes had a tip resistance of 2-5 MΩ. After opening the gigaseal, the microelectrode amplifier (HEKA, EP-10, Lambrecht, Germany) was set to zero holding current and cells were stimulated at a frequency of 0.5 to 1 Hz. Recordings were corrected for a junction potential of 12 mV (32,33). The chemical composition of the external and pipette solutions is described in supplemental material.

INa activation and inactivation were measured by whole-cell voltage-clamp according to standard protocols (32,33). Cells were held at a membrane potential of −80 mV after which pulses between −120 and +50 mV were applied in 5 mV steps each with a duration of 250 ms. Inactivation was measured from membrane potentials between −120 mV and +50 mV at maximal activation (26,33). In view of the small size of zebrafish myocytes (cell capacitance of 6.7±2.6pF; n=47) it was not necessary to reduce extracellular [Na+] to achieve voltage control; extracellular [Na+] was reduced to 50 mM in studies of neonatal rat ventricular myocytes Access resistance of the patch electrodes was <7 MΩ. Measurements showing incomplete control of membrane potential were eliminated from analysis. The chemical composition of the external and pipette solutions is described in supplemental information.

The inward rectifier current IK1 was measured using the protocol described by Dobrev et al. and Nemtsas et al. (34,35). Ramp clamps of 1400 ms duration were applied between −120 and +20 mV. After each clamp, external solution containing Ba2+(1 mM) was applied via a large pore pipette placed adjacent to the clamped cell. IK1 was defined as the total current minus the Ba2+-insensitive current (33). Subtraction of the two currents was performed using a MatLab script. The chemical composition of the external and pipette solutions is described in supplemental information.

The outward K+ current IKr was measured according to the protocol of Nemtsas et al. (34) using identical extracellular and pipette solutions (see on-line supplement). In brief, IKr was activated by clamping the membrane potential from a holding potential of −50 mV for 1000 ms to different levels in 5 mV steps. IKr was obtained as the tail current upon repolarization to −50 mV after the 1000 ms conditioning steps. Interfering ICa,L was inhibited by nisoldipine (1 μM). Addition of the specific IKr blocker E4031 identified the tail current as IKr (35).

Cytokine expression: Conditioned culture media (after 24 or 48 hr) from cells expressing 2057del2 plakoglobin in the presence or absence of SB216763 (5 μM, 24 hr) were collected, mixed with a cocktail of biotinylated detection antibodies and incubated with nitro-cellulose membranes spotted in duplicate with control and capture antibodies (R&D Systems). Chemiluminescent signal produced at each spot corresponded to the amount of cytokine bound. Conditioned media from non-transfected cultures were assayed for cytokine expression as controls.

Knock-down of SAP97 expression in neonatal rat ventricular myocytes: Control (non-transfected) neonatal rat ventricular myocytes were isolated and plated on collagen-coated chamber slides for 24 hr prior to being infected with anti-SAP97 shRNA lentiviral particles (7 m/ml, Santa Cruz Biotechnology) in a 5 μg/ml polybrene mixture (12 hr, 37°). At 72 hours post-transduction, cultures were fixed in 4% paraformaldehyde and analyzed by immunofluorescence (as described in methods included in the supplemental material). Non-specific shRNA (Santa Cruz Biotechnology) was used as a negative control.

Culture and analysis of iPSC-derived cardiac myocytes: iPSC-cardiac myocyte cultures were grown in RPMI+ B27 medium for 5-14 days, rinsed in serum-free medium, fixed in 4% paraformaldehyde and immunostained. Antibodies included mouse monoclonal anti-plakoglobin (Sigma), anti-Cx43 (Millipore), anti-SAP97 (Santa Cruz Biotechnology) and rabbit polyclonal anti-Nav1.5. Specific immunoreactive signal was detected by laser scanning confocal microscopy. Selected cultures were treated with SB216763 (5 μM) for 24 hr prior to fixation. iPSC-cardiac myocytes derived from unaffected non-mutation carrier siblings of the two probands were subjected to the same protocol and used as controls.

Statistical analysis: Data were analyzed using ANOVA or Student's unpaired t-test, where appropriate. Data are expressed as mean±SE.

Zebrafish histology and electron microscopy: Embryos for histological analysis were fixed in JB4, sectioned and stained with hematoxylin and eosin. Embryos for ultrastructural analysis were processed conventionally for transmission EM. Ultrathin sections were stained with uranyl acetate and lead citrate and viewed in a Philips CM10™ electron microscope at 80 keV. Digital images were acquired and transferred to a Metamorph™ workstation for quantitative offline analysis.

Myocardial glycogen assay: Equal amounts of heart tissue were isolated from cmlc:Gal4 fish (control) and cmlc:2057del2 plakoglobin fish. Tissue was homogenized in glycogen development buffer (Abcam) with Bug Bead Homogenizer and then centrifuged at 12000 rpm for 5 min. 2-8 μl of each sample was pipetted into 96-well plate and adjusted with glycogen hydrolysis buffer to 50 μl. A glycogen standard row was added to each plate. 2 μl of hydrolysis enzyme mix was added to standards and samples, and incubated at room temperature for 30 min before adding 44 μl glycogen development buffer, 2 μl development enzyme mix and 2 μl probe to each well. The plates were then incubated at room temperature for 30 min before measuring OD450 nm with a micro-plate reader. Glycogen concentration was calculated using a standard curve, with volume and dilution factors.

Neonatal rat ventricular myocyte isolation, culture, stretch protocols: Primary cardiac myocyte cultures were prepared from ventricles of 1-day-old Wistar rat pups (Charles River) as previously described (36). Cells were plated on collagen-coated plastic chamber slides at a density of 2.4×105 cells/cm2 and grown for 4 days prior to experimentation. In selected experiments, monolayers grown on collagen-coated silicone membranes were subjected to linear pulsatile stretch using a custom-designed apparatus as described in previous studies (35). Cells were stretched to 110% of resting length at a frequency of 3 Hz for 1-4 hr in serum-free medium. In other experiments cultures were incubated with reagents including pifithrin A (5 μM, 1 hr) and SB216763 (0.1-10 μM for 12 or 24 hr) prior to experimentation. Vehicle controls (DMSO) were included in all experiments.

Viral transfection: A recombinant adenoviral construct expressing 2057del2 plakoglobin was created with the ViraPower Adenoviral Expression™ system (Invitrogen) using the pAd/CMV/V5-DEST vector through LR clonase-mediated recombination. The V5-tag was used to identify the mutant protein and distinguish it from wildtype plakoglobin. Two days post-plating, monolayers were exposed to viral solution (1:130 dilution in serum-free media, 37° C.) for 1 hr after which viral solution was replaced with complete medium in the presence or absence of reagents. Immunofluorescence staining and microscopy in cultured cells: Cultures were rinsed in serum-free medium and fixed in 4% paraformaldehyde at 25° C. for 5 min or in acetone at −20° C. for 10 min. Fixed cells were immunostained with mouse monoclonal anti-plakoglobin (Sigma), anti-N-cadherin (Sigma), anti-Cx43 (Millipore), anti-desmoplakin (Fitzgerald), anti-SAP97 (Santa Cruz Biotechnology) antibodies and anti-Nav1.5 antibodies (rabbit polyclonal). Secondary antibodies included Cy3-conjugated goat anti-mouse or anti-rabbit IgGs (H+L) and a FITC-conjugated goat anti-mouse IgG (Jackson ImmunoResearch). Double labeling experiments were conducted with concurrent use of the two primary or secondary antibodies (from different animal species). Specific immunoreactive signal was detected by laser scanning confocal microscopy as previously described (4).

Immunofluorescence staining and microscopy in patient myocardium: Formalin-fixed, paraffin-embedded blocks of right ventricular myocardium were analyzed in eight patients with ARVC. The disease was confirmed pathologically at autopsy in seven patients and by endomyocardial biopsy in one. A desmosomal gene mutation had been identified in six of these patients (desmoplakin: R1113X or 1218+1G→A; plakophilin2: 2506delA or 2146-1G→C; plakoglobin: S39_K40insS; desmoglein2: C591X), whereas in the remaining two cases no mutation was identified in any of the candidate genes screened but histology was consistent with ARVC. A second set of samples came from the native hearts of patients who had undergone cardiac transplantation. Transmural sections of right and left ventricles from each of five patients with end-stage hypertrophic, dilated, or ischemic cardiomyopathies were analyzed. Controls consisted of myocardium obtained at autopsy from four patients in whom there was no clinical or pathological evidence of heart disease. In preparation for immunofluorescence microscopy, deparaffinized, rehydrated slide-mounted sections were heated in citrate buffer (10 mM, pH 6.0), and after being cooled to room temperature, were simultaneously permeabilized and blocked in phosphate-buffered saline (PBS) containing 1% Triton X-100, 3% normal goat serum and 1% bovine serum albumin. The sections were then incubated with a primary anti-SAP97 antibody (rabbit polyclonal, AbCam) and thereafter with indocarbocyanine-conjugated goat anti-rabbit IgG. Immuno-stained preparations were analyzed by confocal microscopy (Zeiss, LSM-510™) as described previously (4).

Western Immunoblotting: Cells were washed in PBS and scraped from culture dishes in a low ionic strength buffer containing protease inhibitors (1 mM NaHCO3, 5 mM EDTA, 1 mM EGTA, 1 μM leupeptin, 1 μM pepstatin, 0.1 μM aprotinin, 1 mM benzamidine, 1 mM iodoacetamide, 1 mM phenylmethylsulfonylfluoride). Following centrifugation, the pellet was resuspended in the same buffer and aliquots containing 10 μg of total protein were analyzed by SDS polyacrylamide gel electrophoresis. Proteins were detected by enhanced chemiluminescence (ECL, Amersham Corp). All blots were stripped in 62.5 mM Tris-HCl, pH 6.8, 100 mM β-mercapto-ethanol, and re-probed with a mouse monoclonal anti-GAPDH antibody (Fitzgerald) as a loading control.

Caspase-3 assay: Caspase-3 activity was assessed by a colorimetric assay (Calbiochem). Control myocytes and myocytes expressing 2057del2 plakoglobin were subjected to 4 hr of cyclical, uniaxial stretch (110% of resting length at 3 Hz), washed in PBS and collected by trypsinization followed by centrifugation. The cellular pellet was resuspended in cell lysis buffer and incubated on ice for 10 min. Lysates were centrifuged for 5 min at 13,000 g, and the supernates were assayed for caspase-3 activity in assay buffer (50 mM HEPES, pH 7.4, 100 mM NaCl, 0.1% 3-[(3-cholamidopropyl) dimethylammonio]-1-propanesulfonate, 10 mM dithiothreitol, 0.1 mM EDTA, 10% glycerol). After addition of the caspase substrate DEVC (2 nM), samples were incubated for 60 min at 37° C. and read at 405 nm (Bio-Tek Instruments). Cultures not subjected to stretch were also assayed for caspase-3 activity as controls.

TUNEL assay: Apoptotic cells in cultures were detected by terminal deoxynucleotidyl transferase-mediated biotinylated UTP nick end-labeling (TUNEL) assay using the ApopTag Fluorescein Direct in Situ Apoptosis Detection KitTM (Millipore) according to the manufacturer's instructions. Nuclei were stained with DAPI; TUNEL-positive and total nuclei were counted under a laser scanning confocal microscope in 5 fields per chamber or silicone membrane in stretch experiments.

Cellular Electrophysiology Solutions: Measurements of INa in zebrafish ventricular myocytes: Composition of extracellular solution (mmoles/Liter): 120 Na+, 5.4 K+, 1.8 Ca2+, 1.1 Mg2+, 131.2 Cl—, 5 HEPES, 10 glucose at pH=7.3 (adjusted with NaOH). Composition of pipette solution (mmoles/Liter): 130 Cs+, 1 Ca2+, 1 Mg2+, 70 aspartate, 64 Cl−, 11 EGTA, and 5 Na-ATP, at pH=7.3 (adjusted with CsOH).

Measurements of INa in neonatal rat ventricular myocytes: Composition of extracellular solution (mmoles/Liter): 50 Na+, m-methyl-D-glutamine 70; 5.4 K+, 1.8 Ca2+, 1.1 Mg2+, 131.2 Cl−, 5 HEPES, 10 glucose at pH=7.3 (adjusted with NaOH). Composition of pipette solution (mmoles/Liter): 130 Cs+, 1 Ca2+, 1 Mg2+, 70 aspartate, 64 Cl−, 11 EGTA, and 5 Na-ATP, at pH=7.3 (adjusted with CsOH).

Measurements of IK]: Composition of extracellular solution (mmoles/Liter): 120 Na+, 20 K+, 2 Ca2+, 1 Mg2+, 146 Cl−, 10 HEPES, 10 glucose at a pH=7.3 (adjusted with NaOH). Composition of pipette solution (mmoles/Liter): 120 K+, 8 Na+, 2 Ca2+, 5 Mg-ATP, 80 aspartate, 52 Cl−, 2 EGTA, 0.1 Tris-GPT at pH=7.3 (adjusted with KOH).

Measurements of IKr: Composition of extracellular solution (mmoles/Liter): 120 Na+, 5.4 K+, 0.5 Ca2+, 2 Mg2+, 130.4 Cl—, 10 HEPES, 10 glucose at pH=7.3 (adjusted with KOH), 1 mol/1 Nisoldipine. Composition of pipette solution (mmoles/Liter): 120 K+, 8 Na+, 2 Ca2+, 3 Mg-ATP, 80 aspartate, 52 Cl−5 EGTA, at a pH=7.3 (adjusted with KOH).

Preparation of induced pluripotent stem cells from patient blood samples: Blood reprogramming was performed as previously described (37). Briefly, 13 mL of blood was isolated from ACM patients expressing plakophilin-2 mutations (Q617X or 2013delC) as well as control family-matched patients under IRB approval. Blood was stored for 10 hours on ice until peripheral blood mononuclear cell (PBMC) isolation was performed by centrifugation using Ficoll-PaqueTM. Two million PBMCs were then cultured in one well of a 12-well plate for 6 days in blood growth media. 1 x 105 PBMCs were infected with a Sendai virus containing the reprogramming factors (POU5F1, SOX2, KLF4, c-MYC) purchased from Invitrogen's CytoTune® iPS Sendai Reprogramming Kits (Life technologies) overnight. The following day, infected PBMCs were placed into one well of a 6-well plate previously coated with a 1:200 dilution of Matrigel™ (BD Biosciences). For three days, infected PBMCs were cultured in blood media and switched to E6 media (Life technologies) containing 50 ug/L fibroblasts growth factor (FGF) and 0.2 mM sodium butryate for 14 additional days. At day 17 after infection, human induced pluripotent cell (hiPSC) colonies could be observed and cultures were switched to E8 media (Life technologies). At day 20 after infection, hiPSC colonies were picked and expanded into E-well culture plates coated with Matrigel™. Subsequent passages of hiPSC cells were cultured in E8 media on MatrigelTM coated plates under hypoxic conditions.

Immunofluorescent labeling of hiPSCs and iPSC-cardiac myocytes: hiPSC derivation was confirmed by immunofluorescent labeling of hiPSC markers with Tra-1-60 (1:500) and OCT4 (1:100, SC-9081, Santa Cruz) primary antibodies. In addition, iPSC-cardiac myocyte derivation was confirmed using anti-troponin T (1:200 MS-295-P, Thermo-Scientific) and anti-a sarcomeric actin (1:500, MA1-21597, Thermo-Scientific) antibodies. Primary antibodies were incubated overnight at 4oC in phosphate-buffered saline containing 3% bovine serum albumin (BSA) followed by three washes using PBS (5 min each wash). Cells were then labeled with the appropriate secondary antibody diluted 1:400 (Life technologies, A-11037 or Alexa Fluor® 488 Goat Anti-Rabbit IgG H+L, A-11001, Alexa Fluor® 488 Goat Anti-Mouse IgG H+L) for 30 min. After secondary antibody labeling, coverslips were washed 3 times (5 min) and mounted using Faramount Aqueous Mounting Media (S3025, Dako, Carpinteria, Calif.). Images were taken using a Zeiss LSM 510TH confocal microscope.

Cardiac myocyte differentiation: At passage 10, hiPSCs were seeded at 1.2x105 per well in a Matrigel™-coated 6-well plate. hiPSCs were cultured in E8 media until 80% confluence was reached. They were then grown in B27 without insulin (A1895601, Life Technologies) in RPMI supplemented with 6 μM CHIR-99021 (CT99021, Selleckchem) to initiate differentiation. CHIR-99021 treatment continued for two days followed by replacement with RPMI supplemented with B27 without insulin for one day followed by a two day treatment of 5 μM IWR-1 (10161, Sigma) in RPMI supplemented with B27 without insulin. Cultures were then changed to RPMI with B27 containing insulin (17504-044, Life Technologies). After two additional days, iPSC derived cardiac myocyte (hiPSC-CM) beating could be observed.

### REFERENCES

1. Basso C, Corrado D, Marcus F I, Nava A, Thiene G, Arrhythmogenic right ventricular cardiomyopathy. Lancet 373, 1289-1300 (2009).
2. Saffitz J E, The pathobiology of arrhythmogenic cardiomyopathy. Annu. Rev. Pathol. 6, 299-321 (2011).
3. Sen-Chowdhry S, Syrris P, Prasad S K, Hughes S E, Merrifield R, Ward D, Pennell D J, McKenna W J, Left-dominant arrhythmogenic cardiomyopathy: an under-recognized clinical entity. J. Am. Coll. Cardiol. 52, 2175-2187 (2008).
4. Asimaki A, Tandri H, Huang H, Halushka M K, Gautam S, Basso C, Thiene G, Tsatsopoulou A, Protonotarios N, McKenna W J, Calkins H, Saffitz J E, A new diagnostic test for arrhythmogenic right ventricular cardiomyopathy. N. Engl. J. Med. 360, 1075-1084 (2009).
5. Asimaki A, Saffitz J E, Gap junctions and arrhythmogenic cardiomyopathy. Heart Rhythm 9, 992-995 (2012).
6. Sato P Y, Musa H, Coombs W, Guerrero-Sema G, Patino G A, Taffet S M, Isom LL, Delmar M, Loss of plakophilin-2 expression leads to decreased sodium current and slower conduction velocity in cultured cardiac myocytes. Circ. Res. 105, 523-526 (2009).
7. Rizzo S, Lodder E M, Verkerk A O, Wolswinkel R, Beekman L, Pilichou K, Basso C, Remme C A, Thiene G, Bezzina CR, Intercalated disc abnormalities, reduced Na+current density and conduction slowing in desmoglein-2 mutant mice prior to cardiomyopathic changes. Cardiovasc. Res. 95, 409-418 (2012).
8. Noorman M, Hakim S, Kessler E, Groeneweg J A, Cox M G, Asimaki A, van Rijen H V, van Stuijvenberg L, Chkourko H, van der Heyden M A, Vos M A, de Jonge N, van der Smagt J J, Dooijes D, Vink A, de Weger R A, Varro A, de Bakker J M, Saffitz J E, Hund T J, Mohler P J, Delmar M, Hauer R N, van Veen T A, Remodeling of the cardiac sodium channel, connexin43, and plakoglobin at the intercalated disk in patients with arrhythmogenic cardiomyopathy. Heart Rhythm 10, 412-419 (2013).
9. Garcia-Gras E, Lombardi R, Giocondo M J, Willerson J T, Schneider M D, Khoury D S, Marian A J, Suppression of canonical wnt/beta-catenin signaling by nuclear plakoglobin recapitulates phenotype of arrhythmogenic right ventricular cardiomyopathy. J. Clin. Invest. 116, 2012-2021 (2006).
10. Ruiz P, Brinkmann V, Ledermann B, Behrend M, Grund C, Thalhammer C, Vogel F, Birchmeier C., Gunthert U, Franke W W, Birchmeier W, Targeted mutation of plakoglobin in mice reveals essential functions of desmosomes in embryonic heart. J. Cell. Biol. 135, 215-225 (1996).
11. Gallicano G I, Kouklis P, Bauer C, Yin M, Vasioukhin V, Degenstein L, Fuchs E, Desmoplakin is required early in development for assembly of desmosomes and cytoskeletal linkage. J. Cell Biol. 143, 2009-2022 (1998).
12. Grossman K S, Grund C, Huelsken J, Behrend M, Erdmann B, Franke W W, Birchmeier W, Requirement of plakophilin2 for heart morphogenesis and cardiac junction formation. J. Cell Biol. 167, 149-160 (2004).
13. Panakova D, Werdich A A, Macrae C A, Wntl 1 patterns a myocardial electrical gradient through regulation of the L-type Ca(2+) channel. Nature 466, 874-878 (2010).
14. Becker J R, Deo R C, Werdich A A, Panàkovà D, Coy S, MacRae C A, Human cardiomyopathy mutations induce myocyte hyperplasia and active hypertrophic pathways during cardiogenesis in zebrafish. Dis. Model Mech. 4, 400-410 (2011).
15. McKoy G, Protonotarios N, Crosby A, Tsatsopoulou A, Anastasakis A, Coonar A, Norman M, Baboonian C, Jeffery S, McKenna W J, Identification of a deletion in plakoglobin in arrhythmogenic right ventricular cardiomyopathy with palmoplantar keratoderma and woolly hair (Naxos disease). Lancet 355, 2119-2124 (2000).
16. Protonotarios N, Tsatsopoulou A, Naxos disease and carvajal syndrome: Cardiocutaneous disorders that highlight the pathogenesis and broaden the spectrum of arrhythmogenic right ventricular cardiomyopathy. Cardiovasc. Pathol. 13, 185-194 (2004).
17. Becker J R, Robinson T Y, Sachidanandan C, Kelly A E, Coy S, Peterson R T, MacRae C A, In vivo natriuretic peptide reporter assay identifies chemical modifiers of hypertrophic cardiomyopathy signalling. Cardiovasc. Res. 93, 463-470 (2012).
18. Eldar-Finkelman H, Martinez A. GSK-3 inhibitors: Preclinical and clinical focus on CNS. Front. Molec. Neurosci. 4, 1-18 (2011).
19. Mallat Z, Tedgui A, Fontaliran F, Frank R, Durigon M, Fontaine G, Evidence of apoptosis in arrhythmogenic right ventricular dysplasia. N. Engl. J. Med. 335, 1190-1196 (1996).
20. Asimaki A, Tandri H, Duffy E R, Winterfield J R, Mackey-Bojack S, Picken M M, Cooper L T, Wilber D J, Marcus F I, Basso C, Thiene G, Tsatsopoulou A, Protonotarios N, Stevenson W G, McKenna W J, Gautam S, Remick D G, Calkins H, Saffitz J E, Altered desmosomal proteins in granulomatous myocarditis and potential pathogenic links to arrhythmogenic right ventricular cardiomyopathy. Circ. Arrhythm. Electrophysiol. 4, 743-752 (2011).
21. Milstein M L, Musa H, Balbuena D P, Anumonwo J M, Auerbach D S, Furspan P B, Hou L, Hu B, Schumacher S M, Vaidyanathan R, Martens J R, Jalife J, Dynamic reciprocity of sodium and potassium channel expression in a macromolecular complex controls cardiac excitability and arrhythmia. Proc. Natl. Acad. Sci. USA 109, E2134-2143 (2012).
22. Zhang S S, Shaw R M, Trafficking highways to the intercalated disc: new insights unlocking the specificity of connexin43 localization. Cell Commun. Adhes. 21, 43-54 (2014)
23. Kaplan S R, Gard J J, Protonotarios N, Tsatsopoulou A, Spiliopoulou C, Anastasakis A, Squarcioni CP, McKenna W J, Thiene G, Basso C, Brousse N, Fontaine G, Saffitz J E, Remodeling of myocyte gap junctions in arrhythmogenic right ventricular cardiomyopathy due to a deletion in plakoglobin (Naxos disease). Heart Rhythm 1, 3-11 (2004).
24. Yamada K, Green K G, Samarel A M, Saffitz J E, Distinct pathways regulate expression of cardiac electrical and mechanical junction proteins in response to stretch. Circ. Res. 97, 346-353 (2005).
25. James C A, Bhonsale A, Tichnell C, Murray B, Russell S D, Tandri H, Tedford R J, Judge D P, Calkins H, Exercise increases age-related penetrance and arrhythmic risk in arrhythmogenic right ventricular dysplasia/cardiomyopathy-associated desmosomal mutation carriers. J. Am. Coll. Cardiol. 62, 1290-1297 (2013).
26. Petitprez S, Zmoos A F, Ogrodnik J, Balse E, Raad N, El-Haou S, Albesa M, Bittihn P, Luther S, Lehnart S E, Hatem S N, Coulombe A, Abriel H, SAP97 and dystrophin macromolecular complexes determine two pools of cardiac sodium channels Nav1.5 in cardiomyocytes. Circ. Res. 108, 294-304 (2011).
27. Gutman G A, Chandy K G, Grissmer S, Lazdunski M, McKinnon D, Pardo LA, Robertson G A, Rudy B, Sanguinetti M C, Stuhmer W, Wang X, International union of pharmacology. LIII. Nomenclature and molecular relationships of voltage-gated potassium channels. Pharmacol. Rev. 57, 473-508 (2005).
28. Ai Z, Fischer A, Spray D C, Brown A M, Fishman G I, Wnt-1 regulation of connexin43 in cardiac myocytes. J. Clin. Invest. 105, 161-171 (2000).
29 Kimmel C B, Ballard W W, Kimmel S R, Ullmann B, Schilling T F, Stages of embryonic development of the zebrafish. Dev. Dyn. 203, 253-310 (1995).
30. Shin J T, Pomerantsev E V, Mably J D, MacRae C A, High-resolution cardiovascular function confirms functional orthology of myocardial contracitility pathways in zebrafish. Physiol Genomics. 42, 300-309 (2010).
31. Thomas S P, Bircher-Lehmann L, Thomas S A, Zhuang J, Saffitz J E, Kleber A G, Synthetic strands of neonatal mouse cardiac myocytes: Structural and electrophysiological properties. Circ. Res. 87, 467-473 (2000).
32. Neher E. Correction for liquid junction potentials in patch clamp experiments. Methods Enzymol. 207, 123-131 (1992).
33. Gavillet B, Rougier J S, Domenighetti A A, Behar R, Boixel C, Ruchat P, Lehr H A, Pedrazzini T, Abriel H, Cardiac sodium channel Nav1.5 is regulated by a multiprotein complex composed of syntrophins and dystrophin. Circ. Res. 99, 407-414 (2006).
34. Dobrev D, Graf E, Wettwer E, Himmel H M, Hala O, Doerfel C, Christ T, Schuler S, Ravens U, Molecular basis of downregulation of G-protein-coupled inward rectifying K(+) current (I(K,ACh) in chronic human atrial fibrillation: Decrease in Girk4 mRNA correlates with reduced I(K,ACh) and muscarinic receptor-mediated shortening of action potentials. Circulation 104, 2551-2557 (2001).
35. Nemtsas P, Wettwer E, Christ T, Weidinger G, Ravens U, Adult zebrafish heart as a model for human heart? An electrophysiological study. J. Mol. Cell. Cardiol. 2010; 48, 161-171 (2010).
36. Zhuang J, Yamada K A, Saffitz J E, Kleber A G, Pulsatile stretch remodels cell-to-cell communication in cultured myocytes. Circ. Res. 87, 316-322 (2000).
37. Churko J M, Burridge P W, Wu J C, Generation of human iPSCs from human peripheral blood mononuclear cells using non-integrative Sendai virus in chemically defined conditions. Methods Mol. Biol. 1036, 81-88 (2013).

1. A method of treating cardiomyopathy, the method comprising administering a compound of Formula I: 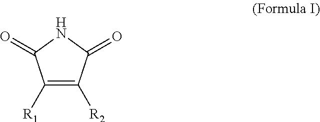
wherein R₁ is optionally substituted heteroaromatic or —NR₂R₃; R₂ is optionally substituted aryl; and R₃ is H or alkyl.

2. The method of claim 1, wherein the cardiomyopathy is selected from the group consisting of:
arrhythmia; arrhythmogenic cardiomyopathy (AC); ischemic heart disease; heart failure;
Naxos disease (ARVC); sarcoidosis; and giant cell myocarditis.

3. The method of claim 2, wherein the cardiomyopathy is arrhythmogenic cardiomyopathy (AC).

4. The method of claim 1 any of claims 1 3, wherein the compound of Formula I is selected from the group consisting of:
SB216763 and SAB415286.

5.-10. (canceled)

11. The method of claim 1, further comprising:
for cardiomyopathy comprising;
measuring/detecting the level of SAP97 polypeptide in a test sample obtained from a subject^{.} and treating the subject with a compound of Formula I: 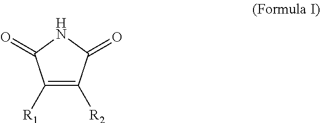
wherein R₁ is optionally substituted heteroaromatic or —NR₂R₃; R₂ is optionally
substituted aryl; and R₃ is H or alkyl,
when the level of SAP97 polypeptide is decreased relative to a reference level.

12. The method of claim 1, wherein the subject is a subject determined to have a level of SAP97 that is decreased relative to a reference level.

13. The method of claim 11, wherein the test sample comprises a cardiomyocyte.

14. The method of claim 12, wherein the level of a SAP97 polypeptide is the level of SAP97 polypeptide in a cardiomyocyte.

15. The method of claim 14, wherein the level of SAP97 polypeptide present in the cardiomyocyte is the level of SAP97 polypeptide located in the membrane of a cardiomyocyte.

16. The assay or method of claim 15, wherein the level of SAP97 polypeptide present in the membrane is the level of SAP97 polypeptide located at cell-cell junctions.

17.-20. (canceled)

21. The method of claim 11, wherein a detectable signal is generated by a SAP97-specific antibody reagent when a SAP97 polypeptide is present.

22.-23. (canceled)

24. The method of claim 11, wherein the polypeptide level is measured using immunochemistry.

25. The method of claim 11, wherein the level of SAP97 polypeptide is normalized relative to the expression level of one or more reference genes or reference proteins.

26. The method of claim 25, wherein the reference level of SAP97 polypeptide is the level of SAP97 polypeptide in a prior sample obtained from the subject.

27. The method of claim 11, further comprising a step of obtaining a sample from the subject.

28.-47. (canceled)

48. An engineered non-human cell expressing 2057del2 plakoglobin.

49. The cell of claim 48, wherein the cell is a cardiomyocyte; a zebrafish cell; or a murine cell.

50.-51. (canceled)

52. A transgenic animal comprising a cell expressing 2057del2plakoglobin.

53. The animal of claim 52, wherein the animal is a zebrafish or a mouse.

54. (canceled)


SEQUENCE LISTING (2 sequences)

SEQ ID NO: 1 - protein, 926 aa, Homo sapiens
MPVRKQDTQRALHLLEEYRSKLSQTEDRQLRSSIERVINIFQSNLFQALIDIQEFYEVTLLDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGQSFNDKRKKNLFSRKFPFYKNKDQSEQETSDADQHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 2 - DNA, 5034 bp, Homo sapiens
gttggaaacggcactgctgagtgaggttgaggggtgtctcggtatgtgcgccttggatctggtgtaggcgaggtcacgcctctcttcagacagcccgagccttcccggcctggcgcgtttagttcggaactgcgggacgcgccggtgggctagggcaaggtgtgtgccctcttcctgattctggagaaaaatgccggtccggaagcaagatacccagagagcattgcaccttttggaggaatatcgttcaaaactaagccaaactgaagacagacagctcagaagttccatagaacgggttattaacatatttcagagcaacctctttcaggctttaatagatattcaagaattttatgaagtgaccttactggataatccaaaatgtatagatcgttcaaagccgtctgaaccaattcaacctgtgaatacttgggagatttccagccttccaagctctactgtgacttcagagacactgccaagcagccttagccctagtgtagagaaatacaggtatcaggatgaagatacacctcctcaagagcatatttccccacaaatcacaaatgaagtgataggtccagaattggttcatgtctcagagaagaacttatcagagattgagaatgtccatggatttgtttctcattctcatatttcaccaataaagccaacagaagctgttcttccctctcctcccactgtccctgtgatccctgtcctgccagtccctgctgagaatactgtcatcctacccaccataccacaggcaaatcctcccccagtactggtcaacacagatagcttggaaacaccaacttacgttaatggcacagatgcagattatgaatatgaagaaatcacacttgaaaggggaaattcagggcttggtttcagcattgcaggaggtacggacaacccacacattggagatgactcaagtattttcattaccaaaattatcacagggggagcagccgcccaagatggaagattgcgggtcaatgactgtatattacgagtaaatgaagtagatgttcgtgatgtaacacatagcaaagcagttgaagcgttgaaagaagcagggtctattgtacgcttgtatgtaaaaagaaggaaaccagtgtcagaaaaaataatggaaataaagctcattaaaggtcctaaaggtcttgggtttagcattgctggaggtgttggaaatcagcatattcctggggataatagcatctatgtaaccaaaataattgaaggaggtgcagcacataaggatggcaaacttcagattggagataaacttttagcagtgaataacgtatgtttagaagaagttactcatgaagaagcagtaactgccttaaagaacacatctgattttgtttatttgaaagtggcaaaacccacaagtatgtatatgaatgatggctatgcaccacctgatatcaccaactcttcttctcagcctgttgataaccatgttagcccatcttccttcttgggccagacaccagcatctccagccagatactccccagtttctaaagcagtacttggagatgatgaaattacaagggaacctagaaaagttgttcttcatcgtggctcaacgggccttggtttcaacattgtaggaggagaagatggagaaggaatatttatttcctttatcttagccggaggacctgctgatctaagtggagagctcagaaaaggagatcgtattatatcggtaaacagtgttgacctcagagctgctagtcatgagcaggcagcagctgcattgaaaaatgctggccaggctgtcacaattgttgcacaatatcgacctgaagaatacagtcgttttgaagctaaaatacatgatttacgggagcagatgatgaatagtagtattagttcagggtcaggttctcttcgaactagccagaagcgatccctctatgtcagagccctttttgattatgacaagactaaagacagtgggcttcccagtcagggactgaacttcaaatttggagatatcctccatgttattaatgcttctgatgatgaatggtggcaagccaggcaggttacaccagatggtgagagcgatgaggtcggagtgattcccagtaaacgcagagttgagaagaaagaacgagcccgattaaaaacagtgaaattcaattctaaaacgagagataaagggcagtcattcaatgacaagcgtaaaaagaacctcttttcccgaaaattccccttctacaagaacaaggaccagagtgagcaggaaacaagtgatgctgaccagcatgtaacttctaatgccagcgatagtgaaagtagttaccgtggtcaagaagaatacgtcttatcttatgaaccagtgaatcaacaagaagttaattatactcgaccagtgatcatattgggacctatgaaagacaggataaatgatgacttgatctcagaatttcctgacaaatttggatcctgtgttcctcatacaactagaccaaaacgagattatgaggtagatggaagagattatcattttgtgacttcaagagagcagatggaaaaagatatccaggaacataaattcattgaagctggccagtataacaatcatctatatggaacaagtgttcagtctgtacgagaagtagcagaaaagggcaaacactgtatccttgatgtgtctggaaatgccataaagagattacagattgcacagctttaccctatctccatttttattaaacccaaatccatggaaaatatcatggaaatgaataagcgtctaacagaagaacaagccagaaaaacatttgagagagccatgaaactggaacaggagtttactgaacatttcacagctattgtacagggggatacgctggaagacatttacaaccaagtgaaacagatcatagaagaacaatctggttcttacatctgggttccggcaaaagaaaagctatgaaaactcatgtttctctgtttctcttttccacaattccattttctttggcatctctttgccctttcctctggagtctttcttgagtactgatttcatgttgaattgtatcccacacatcatggtctgcagcttctttttcacatgtagtgtctccttcaagttacatcgtgtgtattatttaatgtcactattggttagtggccatttttcagaactgaagatggaatggcctgaccagctattaagaacgtggggagacgcagaaattgtggtaaaattccttaatgtttaagggaaagtaactttaagagatttttggaaaagctttatatacattcttttcaaatttcagtacaaatgaaaaagtggttttaatcagtgatttagtagactttgagcaactgtgcacgcttcagtttaatagcatggtttggccagtgtattactctcaagtccttttctcaatcaacttctatcatcaaagcaattgtttcattatagataaataaggacattttttaatttaaaaattcatgtctgagttgactttcataagggatttcattttttcctcaacattcttaaagccttttagtatttgacggttcttttttcccaggacatttgctaggaataacaacgtttcaatgtttttaatctacttgagcaacactatcgtgtcttacaaaagttgttcatatgtaaatgatcatcacatttcgtgaattgaggccatgttcaggtgctaaggaagttcgccttttacacagaagattgagaaaatttcctagatataaatacagataaatcagacgttacagtggtgacgtagtaaccatcatggcaatggaaaggagtccaattcatagcctaaaacttcaaatgtattcttaggagtcagattttactgaatattttacccacaatagctgcctattttgttataataaaatatatataaatatatatataaaactttctttaaactgtaactatgggaattattttctttacatagttgcgcacacacacaaacatatatatatatttaaaatatattttgtttcttttgccacctactcctaactttttgtttggtttttaaattaaatattaattgaatagacttatcttccttaatcatgtgaactgaaaatgggggcatggtggtcatgaggaggaaacatttagggaaattagattataagtaaaagtgtgggcatattctcctcttttctacaaaggttttcaaatggttcctgaggttttttgttgttgtccgtgttgttactgttgttcttgtcatcaggtttgattttggtccttgccctttccttctagttctccttttattaataggaaggcaggcaaaagccccatttatgtgtgtgttttcccctcagacagctttcatccactgctctgcactagaattgcacaaatcttcatggtgagcaattttaagaaatgttagtgaaaggtagaaattatttcacaaatcagtttctctggtccttcatattaataataatatttggcttcccattgctctttggagttgtttattaaatatgtgtttttgacaacctcctcattagtttcttaaatgagtactggtttgtaaagaattatcaacattatccattccatttatgaagaagaggagaacagctaataaactgtattgtaaaatccatatgttaagtgtgtcttgaattttgaaagaaaaaatatattttgcaagctaacattttcttgaaacaatttgaggcatcatgtaacttataaccgaatccaagagccgttaggcagcagagtgtgttaccacattgaaatacacagtgctgctgttagactaaatgtcgtaggttgttaaccacatagaaacacactagtatgaagaaaactgttgtaaaatctcaagagcttcagaaactgccttacaagaccgcagcataagctattttgaagtatttaccaaatagtcacatgttgtaaaatatcaagtggttataaaaggatgccatttatatattaaaatttacataacattgttttctgga